(19) United States
(12) Patent Application Publication
(10) Pub. No.: US 2024/0060030 A1
(43) Pub. Date: Feb. 22, 2024

(54) CELL EXPANSION SYSTEM

(71) Applicant: Terumo BCT, Inc., Lakewood, CO (US)

(72) Inventors: Dalip SETHI, Thronton, CO (US); James ARTHUR, Denver, CO (US); David Alan HANNA, Lakewood, CO (US); Daniel CLEMENT, Lakewood, CO (US); Brian J. NANKERVIS, Golden, CO (US); Jerome HEINTZ, Allenspark, CO (US); Nathan D. FRANK, Arvada, CO (US); Brett BAEVERSTAD, Erie, CO (US); Dennis J. HLAVINKA, Arvada, CO (US)

(21) Appl. No.: 18/233,146

(22) Filed: Aug. 11, 2023

Related U.S. Application Data
(60) Provisional application No. 63/405,998, filed on Sep. 13, 2022.
(60) Provisional application No. 63/399,524, filed on Aug. 19, 2022.

(51) Int. Cl. C12M 1/00; C12M 3/04; C12M 1/36

(52) U.S. Cl. CPC C12M 29/18; C12M 27/10; C12M 29/16; C12M 23/14; C12M 23/28; C12M 41/48

(57) ABSTRACT

Embodiments described herein generally provide for expanding cells in a cell expansion system. The cells may be grown in a bioreactor, and the cells may be activated by an activator (e.g., a soluble activator complex). Nutrient and gas exchange capabilities of a closed, automated cell expansion system may allow cells to be seeded at reduced cell seeding densities, for example. Parameters of the cell growth environment may be manipulated to load the cells into a particular position in the bioreactor for the efficient exchange of nutrients and gases. System parameters may be adjusted to shear any cell colonies that may form during the expansion phase. Metabolic concentrations may be controlled to improve cell growth and viability. Cell residence in the bioreactor may be controlled. In embodiments, the cells may include T cells. In further embodiments, the cells may include T cell subpopulations, including regulatory T cells (Tregs), for example.

### CROSS-REFERENCE TO RELATED APPLICATIONS

This application claims the benefit of U.S. Provisional Application No. 63/399,524 filed on Aug. 19, 2022, and U.S. Provisional Application No. 63/405,998 filed on Sep. 13, 2022. The entire disclosure of the above applications is incorporated herein by reference.

### FIELD

The present disclosure relates to cell expansion systems.

### BACKGROUND

This section provides background information related to the present disclosure which is not necessarily prior art.

Cell Expansion Systems (CESs) are used to expand and differentiate cells. Cell expansion systems may be used to expand, e.g., grow, a variety of adherent and suspension cells. Cells, of both adherent and non-adherent type, may be grown in a bioreactor in a cell expansion system.

### SUMMARY

This section provides a general summary of the disclosure and is not a comprehensive disclosure of its full scope or all of its features.

In at least one example embodiment, the present disclosure provides a method. The method includes loading a first volume of fluid comprising a plurality of cells into the cell expansion system. The cell expansion system includes a cell growth chamber. The method also includes loading a second volume of fluid comprising media into a portion of a first fluid circulation path to position the first volume of fluid in a first portion of the cell growth chamber; feeding the cells by continuously circulating media about an intracapillary circulation loop, an extracapillary circulation loop, or a combination thereof; and expanding the cells.

In at least one example embodiment, the method includes continuously harvesting the cells.

In at least one example embodiment, fluid in the first fluid circulation path flows through an intracapillary space of the cell growth chamber.

In at least one example embodiment, fluid in a second fluid circulation path flows through an extracapillary space of the cell growth chamber.

In at least one example embodiment, the first volume of fluid comprising the plurality of cells is loaded without activating an intracapillary circulation pump.

In at least one example embodiment, the first volume of fluid is the same as the second volume of fluid.

In at least one example embodiment, the present disclosure provides a method of controlling a cell expansion system. The method includes receiving, by a controller, data readouts from at least one sensor; tracking and logging events, with a controller, during a cell expansion process; recording, by the controller, data readouts from the at least one sensor; and reporting, by the controller, events and data readouts on a display.

In at least one example embodiment, the present disclosure provides a method of controlling a fleet of cell expansion devices. The method includes generating, by a controller, a set of instructions for the fleet of cell expansion devices. The fleet of cell expansion devices includes two or more cell expansion devices. The method also includes sending, by the controller, the set of instructions for the fleet of cell expansion devices to each of the cell expansion devices in the fleet of cell expansion devices simultaneously and programming, by the controller, each of the cell expansion devices in the fleet of cell expansion devices with the set of instructions.

In at least one example embodiment, the present disclosure provides a method of controlling a cell expansion system. The method includes actuating, with a controller, a first pump to flow a first fluid at a first fluid flow rate and actuating, with a controller, a second pump to flow a second fluid at a second fluid flow rate. The first fluid flow rate and the second fluid flow rate are opposite.

In at least one example embodiment, the first fluid flow rate and the second fluid flow rate is less than 0.1 mL/min.

In at least one example embodiment, the first fluid flow rate and the second fluid flow rate is 0.01 mL/min.

In at least one example embodiment, the present disclosure provides a media bag for a cell expansion system. The media bag includes a flexible housing defining an internal space configured to house fluid and a pair of ports disposed parallel within a base of the flexible housing. The flexible housing defines a hanger on a top of the internal space, opposite the pair of ports, and the pair of ports includes an inlet port and an outlet port.

In at least one example embodiment, the hanger includes a slot configured to receive one or more hooks for suspending the media bag.

In at least one example embodiment, the media bag includes a rod. The hanger defines a channel in the housing for receiving the rod and the rod and hanger are configured to fit within an external rail for suspending the media bag.

In at least one example embodiment, the present disclosure provides a cell expansion system. The cell expansion system includes a cell expansion device including a housing and a holder. The housing defines a receiver on an operational face thereof and the holder projects from the housing. The cell expansion system also includes a disposable set configured to be engaged with the receiver on the cell expansion device and a media bag configured to be suspended on the holder of the cell expansion device. The media bag has a flexible housing defining an internal space configured to house fluid. The flexible housing defines a hanger on a top of the internal space and the hanger includes a rod extending longitudinally along the top of the media bag. The rod is configured to be received within a channel on the holder to secure the media bag to the holder on the cell expansion device.

In at least one example embodiment, the disposable set includes a cell growth chamber and tubing. The tubing is configured to connect the cell growth chamber to the media bag.

In at least one example embodiment, the holder includes at least one projection and the hanger defines slots configured to receive the projection and suspend the media bag.

In at least one example embodiment, the present disclosure provides a method of controlling a cell expansion system. The method includes actuating, with a controller, a first pump to flow a first fluid at a first fluid flow rate; actuating, with a controller, a second pump to flow a second fluid at a second fluid flow rate; detecting, with a sensor, a parameter related to the first fluid or the second fluid; receiving, by a controller, the parameter from the sensor and determining whether the parameter is outside of a predetermined range; and sending, by a controller, a remote alarm to an external device separate from the cell expansion system.

In at least one example embodiment, the remote alarm includes an email, a text message, a digital alert, or a combination thereof.

In at least one example embodiment, the method includes activating an audio alarm, a visual alarm, or a combination thereof.

In at least one example embodiment, the sensor includes a thermistor, pressure sensor, gas sensor, air detector, or combination thereof.

In at least one example embodiment, the present disclosure provides a method of controlling a cell expansion system. The method includes detecting a parameter related to a cell expansion device; receiving, by a controller, the parameter and determining whether the parameter is outside of a predetermined range; and sending, by a controller, a remote alarm to an external device separate from the cell expansion system.

In at least one example embodiment, the remote alarm includes an email, a text message, a digital alert, or a combination thereof.

In at least one example embodiment, the method includes activating an audio alarm, a visual alarm, or a combination thereof.

In at least one example embodiment, the parameter is one of a temperature, a door position, a pressure, a flow rate, and a concentration.

### DRAWINGS

The drawings described herein are for illustrative purposes only of selected embodiments and not all possible implementations and are not intended to limit the scope of the present disclosure.

FIG. 1A depicts an embodiment of a cell expansion system (CES) according to at least one example embodiment.

FIG. 1B illustrates a front elevation view of an embodiment of a bioreactor showing circulation paths through the bioreactor according to at least one example embodiment.

FIG. 1C illustrates a perspective view of a first bioreactor and a second bioreactor according to at least one example embodiment.

FIG. 1D depicts a rocking device for moving a cell growth chamber rotationally or laterally during operation of a cell expansion system according to at least one example embodiment.

FIG. 2A illustrates a front, perspective view of a cell expansion system according to at least one example embodiment.

FIG. 2B illustrates an interior, perspective view of the cell expansion system of FIG. 2A with a premounted fluid conveyance device according to at least one example embodiment.

FIG. 2C illustrates a rail system and a hook system of a holder of the cell expansion system of FIG. 2A according to at least one example embodiment.

FIG. 3 depicts a perspective view of a housing of a cell expansion system according to at least one example embodiment.

FIG. 4A illustrates a perspective view of a premounted fluid conveyance device according to at least one example embodiment.

FIG. 4B illustrates the premounted fluid conveyance assembly of FIG. 4A according to at least one example embodiment.

FIG. 4C illustrates the premounted fluid conveyance assembly of FIG. 4A according to at least one example embodiment.

FIG. 4D illustrates a media bag of the premounted fluid conveyance assembly of FIG. 4A according to at least one example embodiment.

FIG. 4E illustrates a waste bag of the premounted fluid conveyance assembly of FIG. 4A according to at least one example embodiment.

FIG. 4F illustrates a cross-section view of a pressure pod of the premounted fluid conveyance assembly of FIG. 4A according to at least one example embodiment.

FIG. 4G illustrates an exploded view of the pressure pod of FIG. 4F according to at least one example embodiment.

FIG. 4H illustrates a sampling coil of the premounted fluid conveyance assembly of FIG. 4A according to at least one example embodiment.

FIG. 4I illustrates an in-line filter of the premounted fluid conveyance assembly of FIG. 4A according to at least one example embodiment.

FIG. 5A depicts a schematic of a cell expansion system, including an operational configuration showing fluid movement, according to at least one example embodiment.

FIG. 5B depicts a schematic of a cell expansion system, including another operational configuration showing fluid movement, in accordance with an embodiment of the present disclosure.

FIG. 5C depicts a schematic of a cell expansion system, including another operational configuration showing fluid movement according to at least one example embodiment.

FIG. 6 illustrates a schematic of a cell expansion system according to at least one example embodiment.

FIG. 7A illustrates a schematic of a cell expansion system according to at least one example embodiment.

FIG. 7B illustrates a schematic of a cell expansion system according to at least one example embodiment.

FIG. 7C illustrates a schematic of a cell expansion system according to at least one example embodiment.

FIG. 8 depicts a flow diagram illustrating the operational characteristics of a process for expanding cells according to at least one example embodiment.

FIG. 9A illustrates a flow diagram depicting the operational characteristics of a process for expanding cells according to at least one example embodiment.

FIG. 9B depicts a schematic of a portion of a cell expansion system according to at least one example embodiment.

FIG. 10A depicts a flow diagram illustrating the operational characteristics of a process for expanding cells according to at least one example embodiment.

FIG. 10B illustrates a graph of oxygen consumption in a cell expansion system according to at least one example embodiment.

FIG. 11A illustrates a flow diagram depicting the operational characteristics of a process for expanding cells according to at least one example embodiment.

FIG. 11B illustrates a table with example pump rates that may be used in a cell expansion system according to at least one example embodiment.

FIG. 12A depicts a flow diagram illustrating the operational characteristics of a process for expanding cells according to at least one example embodiment.

FIG. 12B illustrates a graph of the metabolism of expanding cells according to at least one example embodiment.

FIG. 12C illustrates a graph of the metabolism of expanding cells according to at least one example embodiment.

FIG. 13 illustrates a flow diagram depicting the operational characteristics of a process for expanding cells according to at least one example embodiment.

FIG. 14 depicts a flow diagram illustrating the operational characteristics of a process for expanding cells according to at least one example embodiment.

FIG. 15 illustrates a flow diagram depicting the operational characteristics of a process for expanding cells according to at least one example embodiment.

FIG. 16A illustrates a flow diagram depicting the operational characteristics of a process for expanding cells according to at least one example embodiment.

FIG. 16B illustrates a graph of cell number versus flow rate during cell expansion according to at least one example embodiment.

FIG. 17 depicts a flow diagram illustrating the operational characteristics of a process for expanding cells according to at least one example embodiment.

FIG. 18A depicts a flow diagram illustrating the operational characteristics of a process for expanding cells according to at least one example embodiment.

FIG. 18B depicts views of cells expanding in a cell expansion system according to at least one example embodiment.

FIG. 18C illustrates a graph showing inner diameters of cell disassociation according to at least one example embodiment.

FIG. 19 illustrates a graph of cell numbers and flow rate versus culture days during cell expansion according to at least one example embodiment.

FIG. 20A illustrates a flow diagram depicting the operational characteristics of a process for operating pumps to expand cells according to at least one example embodiment.

FIG. 20B depicts a schematic of a portion of a cell expansion system according to at least one example embodiment.

FIG. 20C depicts a schematic of a portion of a cell expansion system according to at least one example embodiment.

FIG. 20D depicts a schematic of a portion of a cell expansion system according to at least one example embodiment.

FIG. 21 illustrates a flow diagram depicting the operational characteristics of a process for expanding cells according to at least one example embodiment.

FIG. 22 depicts a flow diagram illustrating the operational characteristics of a process for expanding cells according to at least one example embodiment.

FIG. 23 illustrates a flow diagram depicting the operational characteristics of a process for expanding cells according to at least one example embodiment.

FIG. 24 depicts a flow diagram illustrating the operational characteristics of a process for expanding cells according to at least one example embodiment.

FIG. 25 depicts an example processing system of a cell expansion system upon which embodiments of the present disclosure may be implemented, according to at least one example embodiment.

FIG. 26 illustrates a flow diagram depicting an experimental flow example according to at least one example embodiment.

FIG. 27 is a graph illustrating example results of a coating procedure according to at least one example embodiment.

FIG. 28 is a graph illustrating example results of cells harvested after a coating procedure according to at least one example embodiment.

FIG. 29 is a graph illustrating example results of cells harvested after a coating procedure according to at least one example embodiment.

FIG. 30 is a graph illustrating example results of viral particles measured per day according to at least one example embodiment.

FIG. 31 is a graph illustrating example results of cells harvested after a coating procedure according to at least one example embodiment.

FIG. 32 is a graph illustrating example results of cells harvested after a coating procedure according to at least one example embodiment.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid, but contact other components to manipulate the system (e.g., a peristaltic pump that pumps fluids through flexible tubing by compressing the exterior of the tube).

The term “donor,” as used herein, can mean any person providing a fluid (e.g., whole blood) to the apheresis system. A donor can also be a patient that also provides a fluid to the apheresis system temporarily while the fluid is processed, treated, manipulated, etc. before being provided back to the patient.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any tangible storage and/or transmission medium that participates in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

The term “module” as used herein refers to any known or later developed hardware, software, firmware, artificial intelligence, fuzzy logic, or combination of hardware and software that is capable of performing the functionality associated with that element.

The terms “determine”, “calculate” and “compute,” and variations thereof, as used herein, are used interchangeably and include any type of methodology, process, mathematical operation or technique.

Embodiments of the present disclosure will be described more fully with reference to the accompanying drawings and in connection with apheresis methods and systems. Embodiments below may be described with respect to separating blood components from whole blood. However, the example procedures are provided simply for illustrative purposes. It is noted that the embodiments are not limited to the description below. The embodiments are intended for use in products, processes, devices, and systems for separating any composite liquid. Accordingly, the present disclosure is not limited to separation of blood components from whole blood.

Example embodiments of the present disclosure are generally directed to systems and methods for expanding cells in a cell expansion system (CES). Such expansion may occur through the use of a bioreactor or cell growth chamber, according to embodiments. In an embodiment, such bioreactor or cell growth chamber may comprise a hollow fiber membrane. Such hollow fiber membrane may include a plurality of hollow fibers and may include an extracapillary (EC) and/or intracapillary (IC) space. Embodiments may provide for adherent or non-adherent cells to be grown or expanded in the cell expansion system. For example, non-adherent or suspension cells, such as T cells, or T lymphocytes, or CD3+ selected cells, may be expanded in the system. In embodiments, one or more subpopulations or subsets of T cells may be grown. For example, embodiments may provide methods and systems for expanding regulatory T cells (Tregs) and/or human regulatory T cells (hTregs).

In some example embodiments, methods and systems may be provided for expanding cells in a closed, automated cell expansion system. In an embodiment, such cell expansion system may include a bioreactor or cell growth chamber. In further embodiments, such bioreactor or cell growth chamber may comprise a hollow fiber membrane. The capabilities of such system, such as nutrient and gas exchange capabilities, may allow cells to be seeded at reduced cell seeding densities. Embodiments provide for parameters of the cell growth environment to be manipulated to load or introduce cells into a position in the bioreactor for the efficient exchange of nutrients and gases to the growing cells. For example, in an embodiment, the centralization of cells in the bioreactor may increase cell density.

In example embodiments, non-adherent cell populations, e.g., T cells and/or Treg cells, may be introduced or loaded into a hollow fiber bioreactor, in which the hollow fiber bioreactor may comprise a plurality of hollow fibers. In embodiments, the cells may be exposed to an activator to activate the expansion of the cells in the hollow fiber bioreactor. In an embodiment, a plurality of cells may be introduced into a cell expansion system using a “load cells centrally without circulation” task, for example. Such task may be performed on Day 0 and on Days 4-8, according to an example embodiment. Other days may be used in other embodiments. In embodiments, such loading of cells task may result in the centralization of cells in the bioreactor to increase cell density. In other embodiments, the cells may be located in other portions or regions of the bioreactor to increase cell density. In embodiments, by positioning the cells in a first position, e.g., about a central region, of the bioreactor, the cells may receive an efficient exchange of nutrients and gases.

In example embodiments, a reduced cell seeding density, as compared to cell seeding densities used with static culture methods, may be used. In an embodiment using a cell expansion system, cells, e.g., Tregs or Treg cells, may be expanded from a cell seeding density of 2.54×10⁵ cells/mL to 3.69×10⁵ cells/mL. In other embodiments, the cell seeding density may be less than about 1×10⁶ cells/mL. In addition, a Treg cell inoculum may be prepared from a cell seeding density of 1.0×10⁵ cells/mL. Other methods, e.g., static Treg cell culture methods, may use a cell seeding density of 1.0×10⁶ Treg cells/mL for in vitro expansion. In an embodiment, lower cell seeding densities may be used due to the system's overall efficiency in delivering nutrients to the culture environment, for example. In other embodiments, one or more of the steps used during expansion in combination with the system's overall efficiency in delivering nutrients to the culture environment may allow lower initial cell seeding densities to be used.

In example embodiments, an automated cell, e.g., Treg, expansion may be performed with a soluble activator complex. In other embodiments, other types of activators, such as beads for the stimulation of cells, may be used, for example. In other embodiments, cells, e.g., Treg cells, may be expanded without the use of a bead-based stimulation. In one embodiment, cell expansion may be performed using the Stem Cell Technologies soluble ImmunoCult™ Human CD3/CD28/CD2 T Cell Activator to activate and expand Treg cells in the presence of 200 IU/mL of the cytokine IL-2 with an automated cell expansion system. Using a soluble activator complex may reduce costs for stimulation over the cost of a bead-based protocol, for example Other types of activators may be used in other embodiments. Further, other types of cytokines or other growth factors may be used in other embodiments.

Example embodiments also may provide for system parameters to be adjusted or managed to control cell residence in the bioreactor or cell growth chamber. By controlling cell residence in the bioreactor hollow fibers during the cell growth phase, for example, the system may provide for an efficient gas and nutrient exchange to expanding cells. In embodiments, a bioreactor may be designed to provide gas exchange, and, in some embodiments, nutrient exchange, to growing cells. In an example embodiment, a bioreactor comprising a semi-permeable hollow fiber membrane may provide gas and nutrient exchange through the semi-permeable hollow fiber membrane. In embodiments, methods for providing media constituents, such as various cytokines, proteins, etc., for example, to growing cells which cannot pass through the membrane may use a fluid inlet to the side of the bioreactor, e.g., intracapillary (IC) side, where cells are growing. However, even low, decreased, or reduced, e.g., minimum, inlet flow rates (0.1 mL/min, for example) may result in cells collecting in the outlet header of the bioreactor, according to embodiments. Cells residing in the headers of the bioreactor may not receive proper gas exchange and nutrient exchange, which may result in cell death and aggregation.

Example embodiments relate to providing methods to retain cells, e.g., non-adherent cell populations, in the bioreactor when feeding the cells using an inlet, e.g., IC inlet, flow. While embodiments herein may refer to cells being on the IC side of the membrane when feeding, for example, other embodiments may provide for the cells to be on the EC side of the membrane, in which cells may be contained within a first circulation path and/or a second circulation path, according to embodiments. In embodiments, a feeding method may provide for pumping a first volume of fluid, e.g., media or cell growth formulated media, into a first port of the bioreactor at a first volumetric flow rate, volume flow rate, fluid flow rate, flow rate, rate of fluid flow, or volume velocity, for example. Volumetric flow rate, volume flow rate, fluid flow rate, flow rate, rate of fluid flow, or volume velocity, for example, may be used interchangeably. In some embodiments, flow rate may be a vector having both a speed and a direction. A second volume of the fluid may be pumped into a second port of the bioreactor at a second volumetric flow rate, volume flow rate, fluid flow rate, flow rate, rate of fluid flow, or volume velocity. In embodiments, such volumetric flow rate, volume flow rate, fluid flow rate, flow rate, rate of fluid flow, or volume velocity, may be controlled by one or more pump rate(s) and/or pump flow rate(s), for example. A pump rate may produce, cause, or affect a volumetric flow rate or flow rate of a fluid upon which the pump may act. As used herein, a pump rate may be described in embodiments as the volumetric flow rate or fluid flow rate produced, caused, or affected by the pump.

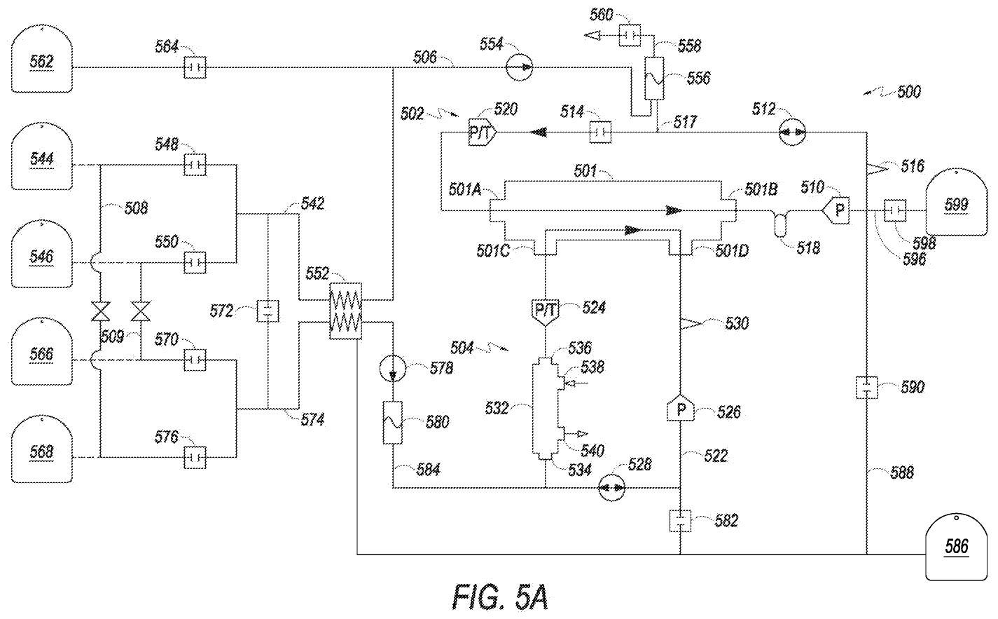

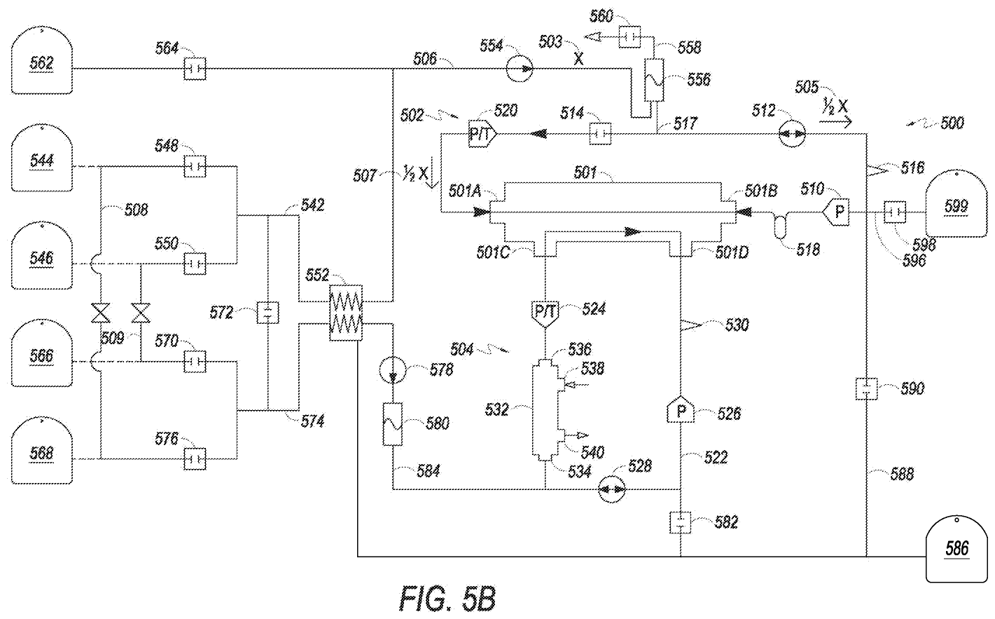

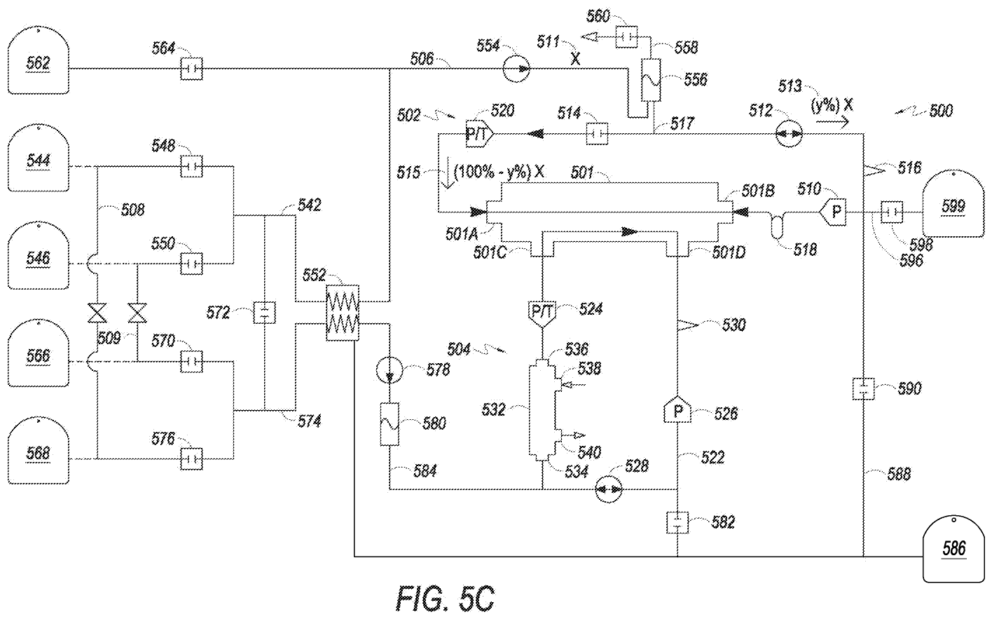

In example embodiments, the second flow rate of the fluid into the bioreactor may be opposite the direction of the first flow rate of the fluid into the bioreactor. For example, FIGS. 5B and 5C illustrate example operational configurations showing flow rates and flow directions that may be used with a cell expansion system, such as CES 500 (e.g., FIGS. 5B, & 5C), in accordance with embodiments of the present disclosure. In embodiments, cell expansion system pumps, e.g., IC pumps, may be used to control cell residence in the bioreactor. In embodiments, cells may be lost from the bioreactor into the IC circulation path or IC loop, for example, during the expansion phase of growth. In embodiments, cells in the bioreactor that are closer to IC inlet port, for example, may receive the freshest growth media, whereas, cells in the portion of the IC circulation path outside of the bioreactor, for example, may essentially be receiving expended or conditioned media which may affect their glycolytic metabolism. In addition, cells in the bioreactor may receive mixed gas (O₂, CO₂, N₂) input from the gas transfer module (GTM) by diffusion from the EC loop circulation, whereas cells in the portion of the IC circulation path outside of the bioreactor may not, according to embodiments.

In example embodiments, reducing the loss of cells from a hollow fiber membrane (HFM) bioreactor may be accomplished by matching, or closely or substantially matching, the IC circulation pump rate to the IC inlet pump rate, but in the opposite direction, during feeding. For example, an IC inlet pump rate of +0.1 mL/min may be matched, or closely or substantially matched, to a complementary IC circulation pump rate of −0.1 mL/min in order to maintain cells in the bioreactor during the growth phase of the cell culture which may be Days 4-7, in embodiments. This pump adjustment may counteract the forces associated with the loss of cells from the IC outlet port, in accordance with embodiments. In other embodiments, other pump rates may be used. For example, in other embodiments, the pump rates may be different. In embodiments, other pumps or additional pumps may be used. In an embodiment, fewer pumps may be used. Further, other time periods may be used in other embodiments.

In example embodiments, the metabolic activity of the cell population may affect feeding parameters. For example, cell culture lactate values may be maintained at or below a predefined level. In an embodiment, cell culture values may be maintained at or below about 7 mmol/L, for example. In embodiments, by using a cell expansion system graphical user interface (GUI) to control a rate(s) of media addition, lactate metabolic waste product from glycolysis may be maintained at or below a predefined value, during the expansion of cells, e.g., regulatory T cells. In other embodiments, rate(s) of media addition, for example, and/or other settings may be controlled to maintain, or attempt to maintain, the lactate levels≤about 5 mmol/L, for example, to improve cell growth and viability. Other concentrations may be used in other embodiments.

Additional embodiments may provide for system features to be harnessed to shear any cell colonies, micro-colonies, or cell clusters that may form during the expansion phase. For example, an embodiment provides for the shearing of colonies, e.g., micro-colonies, of cells through a bioreactor hollow fiber membrane to reduce the number of cells in the micro-colony, colony, or cluster, in which a micro-colony, colony, or cluster may be a group of one or more attached cells. In embodiments, a cell expansion system (CES) bioreactor architecture may be used to shear cell, e.g., Treg cell, micro-colonies. In embodiments, as cells, e.g., Treg cells, grow, they tend to form micro-colonies that may limit the diffusion of nutrients to the cell(s) in the center of the colony. This may lead to adverse effects such as necrosis during cell culture. Embodiments may provide a protocol to shear the colonies by circulating the suspension cell culture through, for example, the hollow fiber Intracapillary (IC) loop (e.g., with hollow fibers of 215 μm inner diameter) during the expansion phase of growth. In embodiments, a colony, micro-colony, or cluster of cells may be sheared to reduce a size of the colony, micro-colony, or cluster of cells. In an embodiment, a colony or cluster of cells may be sheared to provide a single cell suspension and create cell growth/viability. In embodiments, such capabilities may contribute to the continuous perfusion growth of the cells, e.g., T cells or Tregs.

In example embodiments, a therapeutic dose of cells, e.g., Tregs, may be expanded in, and harvested from, a cell expansion system. In embodiments, the number of cells at harvest may be from about 1×10⁶ cells to about 1×10¹⁰ cells, such as on the order of 1×10⁹ cells. In one embodiment, the number of harvested cells may be from about 1×10⁸ and 1×10¹⁰ cells, one example being between about 7.0×10⁸ to about 1.4×10⁹ cells. In embodiments, the harvested cells may have viabilities between about 60% and about 100%. For example, the viability of the harvested cells may be above about 65%, above about 70%, above about 75%, above about 80%, above about 85%, above about 90%, or even above about 95%. The harvested cells may express biomarkers consistent with Tregs, in some embodiments. For example, the cells may express CD4⁺, CD25⁺, and/or FoxP3⁺ biomarkers, in some embodiments. In embodiments, the harvested cells may include the CD4+CD25+ phenotype at a frequency of between about 50% and about 100%. The harvested cells may include the CD4⁺CD25⁺ phenotype at a frequency of above about 75%, above about 80%, above about 85%, above about 90%, or even above about 95%. In other embodiments, the cells may include the CD4⁺FoxP3⁺ phenotype at a frequency of between about 30% to about 100%. In some embodiments, the harvested cells may include the CD4⁺FoxP3⁺ phenotype at a frequency of above about 30%, above about 35%, above about 40%, above about 45%, above about 50%, above about 55%, above about 60%, above about 65%, or even above about 70%.

Example embodiments are directed to a cell expansion system, as noted above. In embodiments, such cell expansion system is closed, in which a closed cell expansion system comprises contents that are not directly exposed to the atmosphere. Such cell expansion system may be automated. In embodiments, cells, of both adherent and non-adherent or suspension type, may be grown in a bioreactor in the cell expansion system. According to embodiments, the cell expansion system may include base media or other type of media. Methods for replenishment of media are provided for cell growth occurring in a bioreactor of the closed cell expansion system. In embodiments, the bioreactor used with such systems is a hollow fiber bioreactor. Many types of bioreactors may be used in accordance with embodiments of the present disclosure.

The system may include, in embodiments, a bioreactor that is fluidly associated with a first fluid flow path having at least opposing ends, a first opposing end of the first fluid flow path fluidly associated with a first port of a hollow fiber membrane and a second end of the first fluid flow path fluidly associated with a second port of the hollow fiber membrane. In embodiments, a hollow fiber membrane comprises a plurality of hollow fibers. The system may further include a fluid inlet path fluidly associated with the first fluid flow path, in which a plurality of cells may be introduced into the first fluid flow path through the first fluid inlet path. In some embodiments, a pump for transferring intracapillary inlet fluid from an intracapillary media bag to the first fluid flow path and a controller for controlling operation of the pump are included. The controller, in embodiments, controls the pump to transfer cells from a cell inlet bag to the first fluid flow path, for example. Another pump for circulating fluid in a first fluid circulation path may also be included, in which such pump may also include a controller for controlling operation of the pump. In an embodiment, a controller is a computing system, including a processor(s), for example. The one or more controller(s) may be configured, in embodiments, to control the one or more pump(s), such as to circulate a fluid at a flow rate within the first fluid circulation path, for example. A number of controllers may be used, e.g., a first controller, second controller, third controller, fourth controller, fifth controller, sixth controller, etc., in accordance with embodiments. Further, a number of pumps may be used, e.g., a first pump, second pump, third pump, fourth pump, fifth pump, sixth pump, etc., in accordance with embodiments of the present disclosure. In addition, while the present disclosure may refer to a media bag, a cell inlet bag, etc., multiple bags, e.g., a first media bag, a second media bag, a third media bag, a first cell inlet bag, a second cell inlet bag, a third cell inlet bag, etc., and/or other types of containers, may be used in embodiments. In other embodiments, a single media bag, a single cell inlet bag, etc., may be used. Further, additional or other fluid paths, e.g., a second fluid flow path, a second fluid inlet path, a second fluid circulation path, etc., may be included in embodiments.

In example embodiments, the system is controlled by, for example: a processor coupled to the cell expansion system; a display device, in communication with the processor, and operable to display data; and a memory, in communication with and readable by the processor, and containing a series of instructions. In embodiments, when the instructions are executed by the processor, the processor receives an instruction to prime the system, for example. In response to the instruction to prime the system, the processor may execute a series of steps to prime the system and may next receive an instruction to perform an IC/EC washout, for example. In response to an instruction to load cells, for example, the processor may execute a series of steps to load the cells from a cell inlet bag, for example, into the bioreactor.

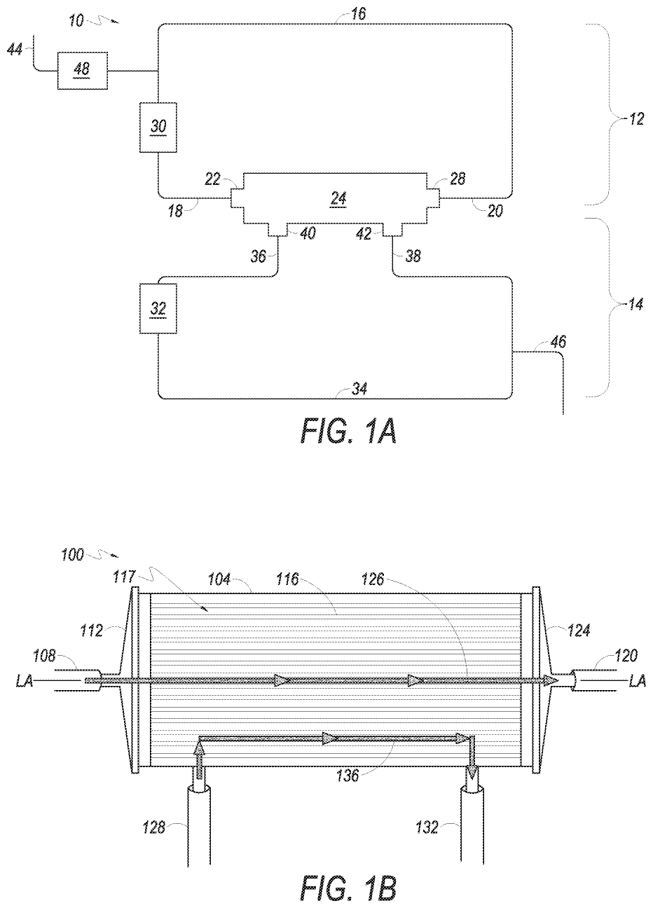

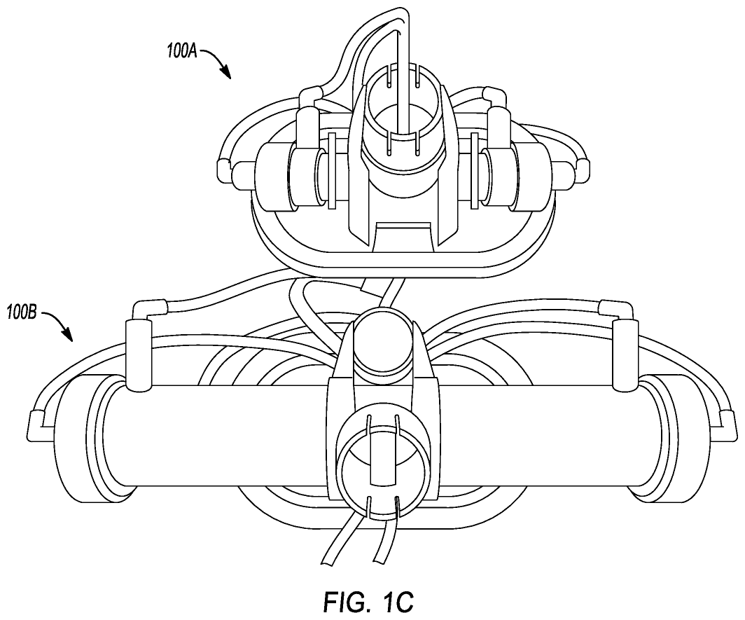

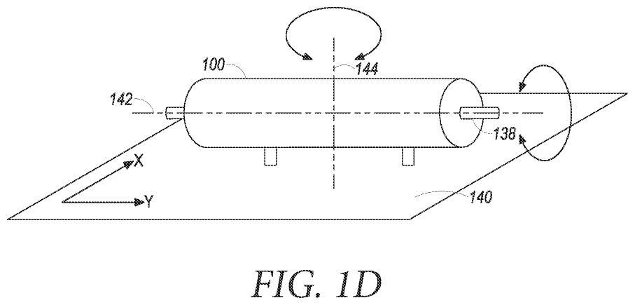

A schematic of an example cell expansion system (CES) is depicted in FIG. 1A, in accordance with embodiments of the present disclosure. CES 10 includes a first fluid circulation path 12 and second fluid circulation path 14. First fluid flow path 16 has at least opposing ends 18 and 20 fluidly associated with a hollow fiber cell growth chamber 24 (also referred to herein as a “bioreactor”), according to embodiments. Specifically, opposing end 18 may be fluidly associated with a first inlet 22 of cell growth chamber 24, and opposing end 20 may be fluidly associated with first outlet 28 of cell growth chamber 24. Fluid in first fluid circulation path 12 flows through the interior of hollow fibers 116 (see FIG. 1B) of hollow fiber membrane 117 (see FIG. 1B) disposed in cell growth chamber 24 (cell growth chambers and hollow fiber membranes are described in more detail infra). Further, first fluid flow control device 30 may be operably connected to first fluid flow path 16 and may control the flow of fluid in first fluid circulation path 12.

Second fluid circulation path 14 includes a second fluid flow path 34, cell growth chamber 24, and a second fluid flow control device 32. The second fluid flow path 34 has at least opposing ends 36 and 38, according to embodiments. Opposing ends 36 and 38 of the second fluid flow path 34 may be fluidly associated with inlet port 40 and outlet port 42 respectively of cell growth chamber 24. Fluid flowing through cell growth chamber 24 may be in contact with the outside of hollow fiber membrane 117 (see FIG. 1B) in the cell growth chamber 24, in which a hollow fiber membrane comprises a plurality of hollow fibers. Second fluid circulation path 14 may be operably connected to second fluid flow control device 32.

First fluid circulation path 12 and second fluid circulation path 14 may thus be separated in cell growth chamber 24 by a hollow fiber membrane 117 (see FIG. 1B). Fluid in first fluid circulation path 12 flows through the intracapillary (“IC”) space of the hollow fibers in the cell growth chamber 24. First fluid circulation path 12 may be referred to as the “IC loop.” Fluid in second circulation path 14 flows through the extracapillary (“EC”) space in the cell growth chamber 24. Second fluid circulation path 14 may be referred to as the “EC loop.” Fluid in first fluid circulation path 12 may flow in either a co-current or counter-current direction with respect to flow of fluid in second fluid circulation path 14, according to embodiments.

Fluid inlet path 44 may be fluidly associated with first fluid circulation path 12. Fluid inlet path 44 allows fluid into first fluid circulation path 12, while fluid outlet path 46 allows fluid to leave CES 10. Third fluid flow control device 48 may be operably associated with fluid inlet path 44. Alternatively, third fluid flow control device 48 may alternatively be associated with fluid outlet path 46.

Fluid flow control devices as used herein may comprise a pump, valve, clamp, or combination thereof, according to embodiments. Multiple pumps, valves, and clamps can be arranged in any combination. In various embodiments, the fluid flow control device is or includes a peristaltic pump. In embodiments, fluid circulation paths, inlet ports, and outlet ports may be constructed of tubing of any material.

Generally, any kind of fluid, including buffers, protein containing fluid, and cell-containing fluid, for example, can flow through the various circulations paths, inlet paths, and outlet paths. As used herein, “fluid,” “media,” and “fluid media” are used interchangeably.

Turning to FIG. 1B, an example of a hollow fiber cell growth chamber 100, or a bioreactor 100, which may be used with the present disclosure is shown in front side elevation view. Cell growth chamber 100 has a longitudinal axis LA-LA and includes cell growth chamber housing 104. In at least one embodiment, cell growth chamber housing 104 includes four openings or ports: IC inlet port 108, IC outlet port 120, EC inlet port 128, and EC outlet port 132.

According to embodiments of the present disclosure, fluid in a first circulation path enters cell growth chamber 100 through IC inlet port 108 at a first longitudinal end 112 of the cell growth chamber 100, passes into and through the intracapillary side (referred to in various embodiments as the intracapillary (“IC”) side or “IC space” of a hollow fiber membrane) of a plurality of hollow fibers 116 comprising hollow fiber membrane 117, and out of cell growth chamber 100 through IC outlet port 120 located at a second longitudinal end 124 of the cell growth chamber 100. The fluid path between the IC inlet port 108 and the IC outlet port 120 defines the IC portion 126 of the cell growth chamber 100. Fluid in a second circulation path flows in the cell growth chamber 100 through EC inlet port 128, comes in contact with the extracapillary side or outside (referred to as the “EC side” or “EC space” of the membrane) of the hollow fibers 116, and exits cell growth chamber 100 via EC outlet port 132. The fluid path between the EC inlet port 128 and the EC outlet port 132 comprises the EC portion 136 of the cell growth chamber 100. Fluid entering cell growth chamber 100 via the EC inlet port 128 may be in contact with the outside of the hollow fibers 116. Small molecules (e.g., ions, water, oxygen, lactate, metabolites, nutrients, gasses, etc.) may diffuse (for example, continuously perfuse) through the hollow fibers 116 from the interior or IC space of the hollow fiber to the exterior or EC space, or from the EC space to the IC space. For example, the hollow fibers 116 may include 200-micron diffusion distances providing more efficient transfer of gas and nutrients as compared to a flask-based system. Large molecular weight molecules, such as growth factors, may be typically too large to pass through the hollow fiber membrane, and may remain in the IC space of the hollow fibers 116. The hollow fibers 116 produce less shear stress when compared to a stir take bioreactor and a wave motion bioreactor. The media may be replaced as needed, in embodiments. Media may also be circulated through an oxygenator or gas transfer module to exchange gasses as needed. Cells may be contained within a first circulation path and/or a second circulation path, as described below, and may be on either the IC side and/or EC side of the membrane, according to embodiments.

The material used to make the hollow fiber membrane 117 may be any biocompatible polymeric material which is capable of being made into hollow fibers. One material which may be used is a synthetic polysulfone-based material, according to an embodiment of the present disclosure.

According to some embodiments, as shown in FIG. 1C, more than one size of the cell growth chamber 100 may be included separately or in a disposables kit. For example, a pair of cell growth chambers 100A and 100B may be included separately or in the disposables kit. In at least one example embodiment, the cell growth chamber 100A may be smaller than the cell growth chamber 100B to accommodate fewer cells for expansion. For example, the cell growth chamber 100A may be a tenth, or 0.1, the size of the cell growth chamber 100B. In at least one example embodiment, the cell growth chamber 100A (or small cell growth chamber 100A) may be advantageous for trial runs, pediatrics and other events (e.g., skin grafts, etc.) with a small batch of starter cells. Use of the small cell growth chamber 100A is advantageous where the initial cell source is too small for the cell growth chamber 100B because the small cell growth chamber 100A can grow the small cell batch and the small cell batch may be transitioned to the cell growth chamber 100B for additional growth. Accordingly, the small cell growth chamber 100A provides an additional opportunity for use of the CES 10 where cell growth chamber 100B may not be optimal. In some example embodiments, the CES 10 may automatically recognize which of the cell growth chamber 100A and the cell growth chamber 100B is installed and identify appropriate process parameters, as described further below. By including more than one size of cell growth chamber 100 the CES 10 may have increased applications including, for example, bone-marrow-derived mesenchymal stem cells (MSCs), adipose-derived MSCs, umbilical cord MSCs, fibroblasts, keratinocytes, HEK293T cells, human embryonic stem cells (ESCs), periosteum-derived cells, induced pluripotent stem-cell-derived MSCs (IPS-MSCs), neural stem cells (NSCs), osteochondro-progenitor cells, endothelial cells, dendritic cells, induced pluripotent stem cells (iPSCs), T cells, viral vectors, exosomes production, expansion of autologous and allogeneic doses of adherent and suspension cell types, etc. For example, the multiple cell growth chambers 100 for the CES 10 provide capabilities to expand within an example range of up to 300 million to 1 billion MSCs or up to 25 billion T cells per run.

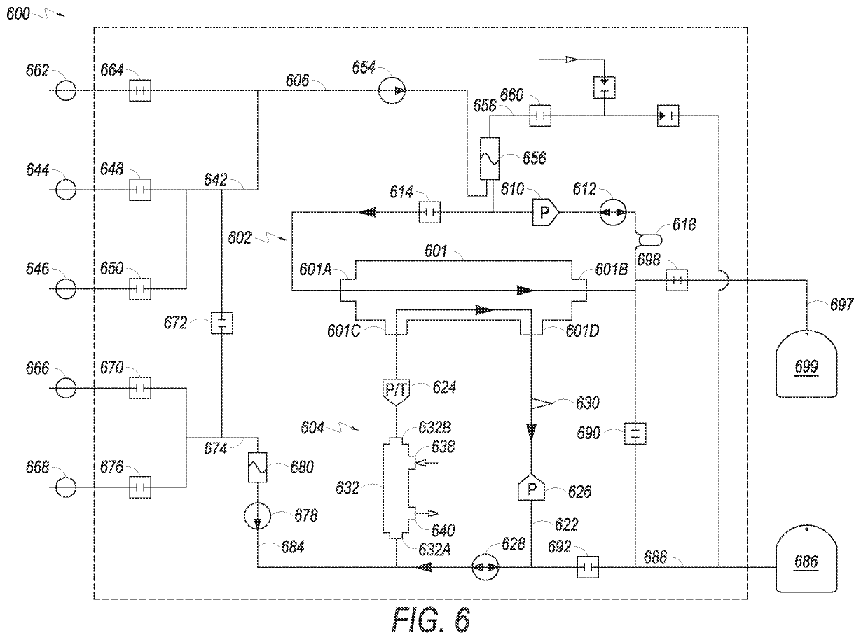

In at least one example embodiment, the CES (such as CES 500 (see FIGS. 5A, 5B, & 5C) and/or CES 600 (see FIG. 6), for example) may include a device configured to move or “rock” the cell growth chamber relative to other components of the cell expansion system by attaching it to a rotational and/or lateral rocking device. FIG. 1D shows one such exemplary device, in which the cell growth chamber 100 may be rotationally connected to two rotational rocking components and to a lateral rocking component, according to an embodiment.

A first rotational rocking component 138 rotates the cell growth chamber 100 around central axis 142 of the cell growth chamber 100. The first rotational rocking component 138 may be rotationally associated with cell growth chamber 100. In some example embodiments, the cell growth chamber 100 may be rotated continuously in a single direction around central axis 142 in a clockwise or counterclockwise direction. Alternatively, the cell growth chamber 100 may rotate in alternating fashion, first clockwise, then counterclockwise, for example, around central axis 142, according to embodiments.

The CES may also include a second rotational rocking component that rotates the cell growth chamber 100 around rotational axis 144. Rotational axis 144 may pass through the center point of the cell growth chamber 100 and may be normal to central axis 142. The cell growth chamber 100 may be rotated continuously in a single direction around rotational axis 144 in a clockwise or counterclockwise direction, in embodiments. Alternatively, the cell growth chamber 100 may be rotated around rotational axis 144 in an alternating fashion, first clockwise, then counterclockwise, for example. In various embodiments, the cell growth chamber 100 may also be rotated around rotational axis 144 and positioned in a horizontal or vertical orientation relative to gravity.

In some example embodiments, the lateral rocking component 140 may be laterally associated with the cell growth chamber 100. The plane of lateral rocking component 140 moves laterally in the −x and −y directions, in embodiments. The settling of cells in the cell growth chamber may be reduced by movement of cell-containing media within the hollow fibers, according to embodiments.

The rotational and/or lateral movement of a rocking device may reduce the settling of cells within the device and reduce the likelihood of cells becoming trapped within a portion of the cell growth chamber. The rate of cells settling in the cell growth chamber is proportional to the density difference between the cells and the suspension media, according to Stokes's Law. In certain embodiments, a 180-degree rotation (fast) with a pause (having a total combined time of 30 seconds, for example) repeated as described above keeps non-adherent red blood cells, for example, suspended. An example embodiment may rotate the cell growth chamber 100 a minimum rotation of about 180 degrees to a maximum of about 290 to 300 degrees; however, one could use rotation of up to 360 degrees or greater. Different rocking components may be used separately, or may be combined in any combination. For example, a rocking component that rotates the cell growth chamber 100 around central axis 142 may be combined with the rocking component that rotates the cell growth chamber 100 around axis 144. Likewise, clockwise and counterclockwise rotation around different axes may be performed independently in any combination.

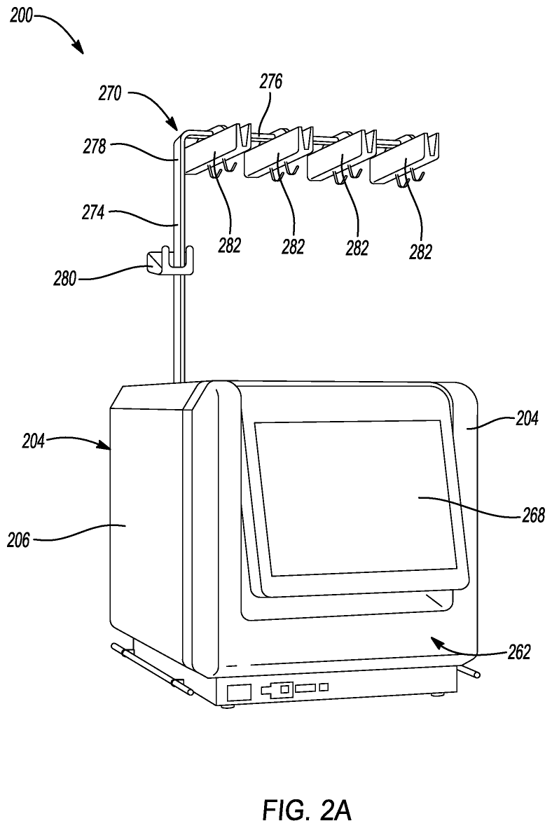

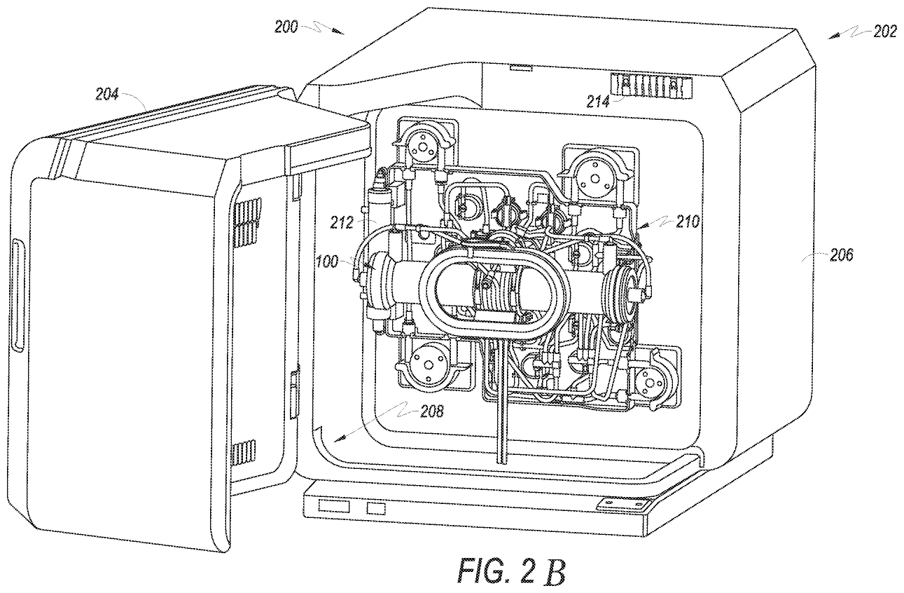

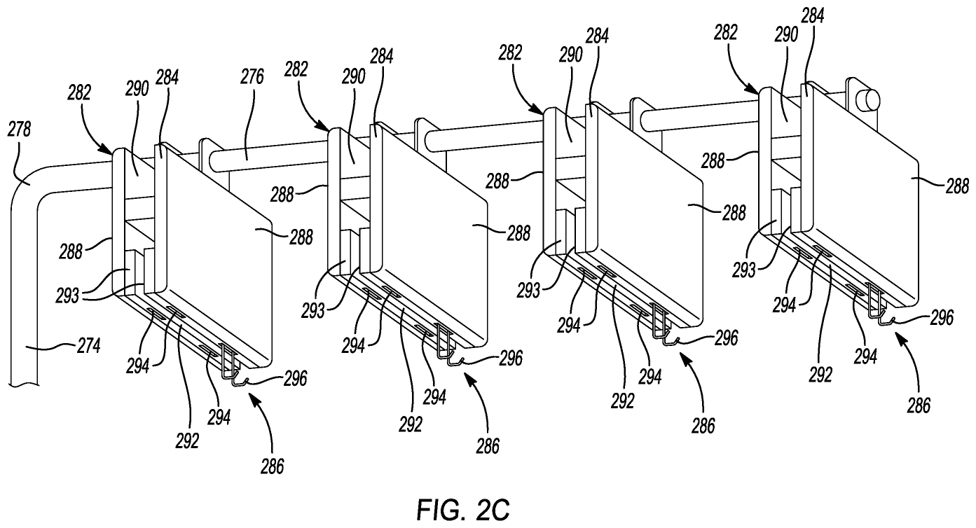

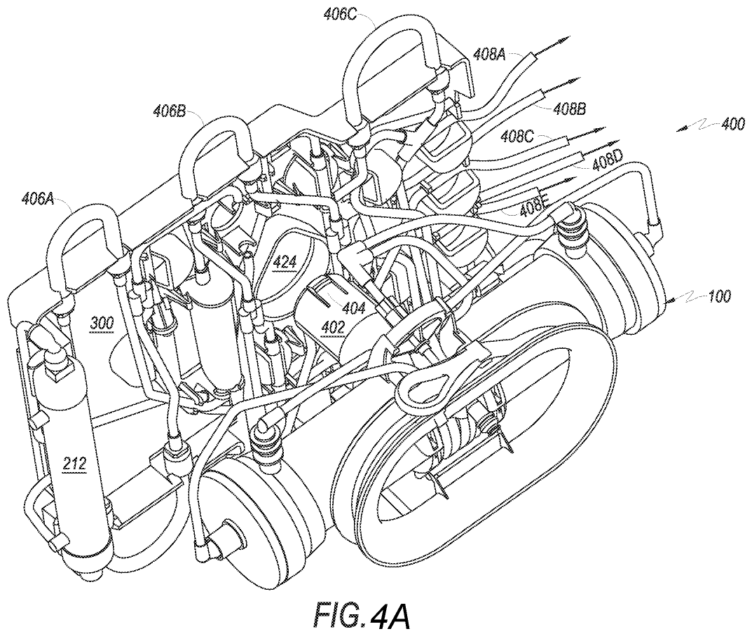

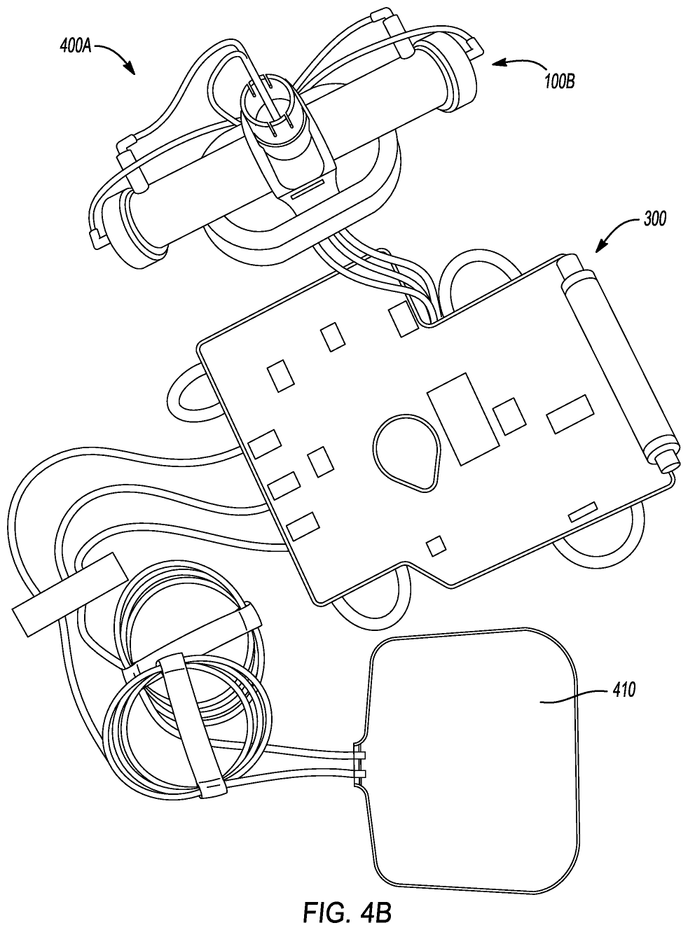

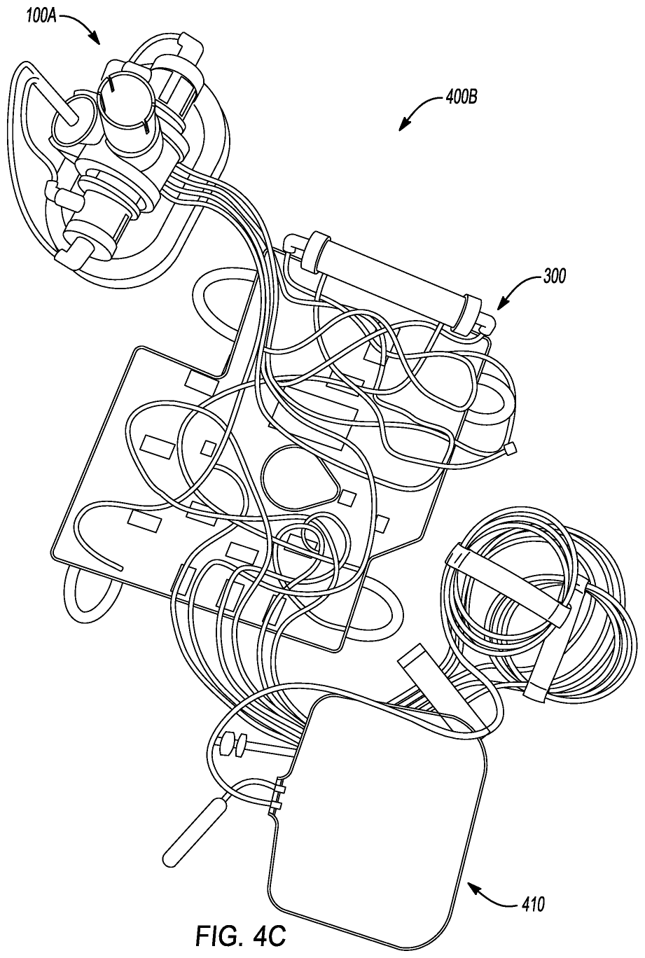

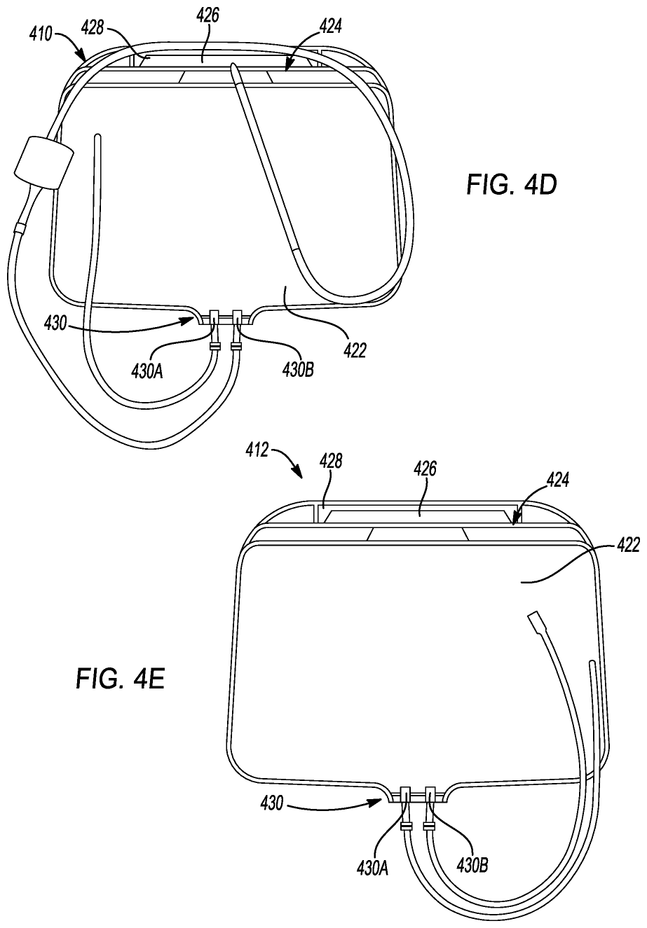

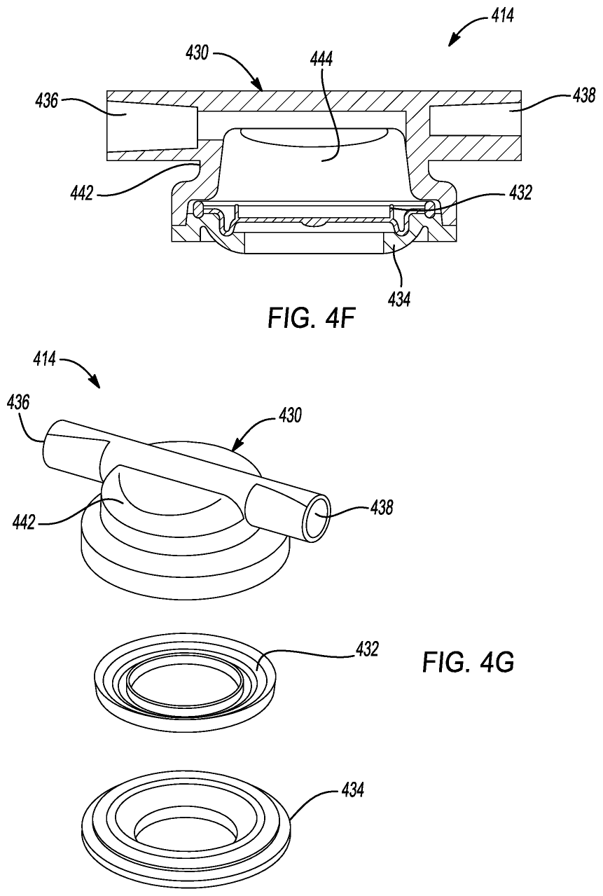

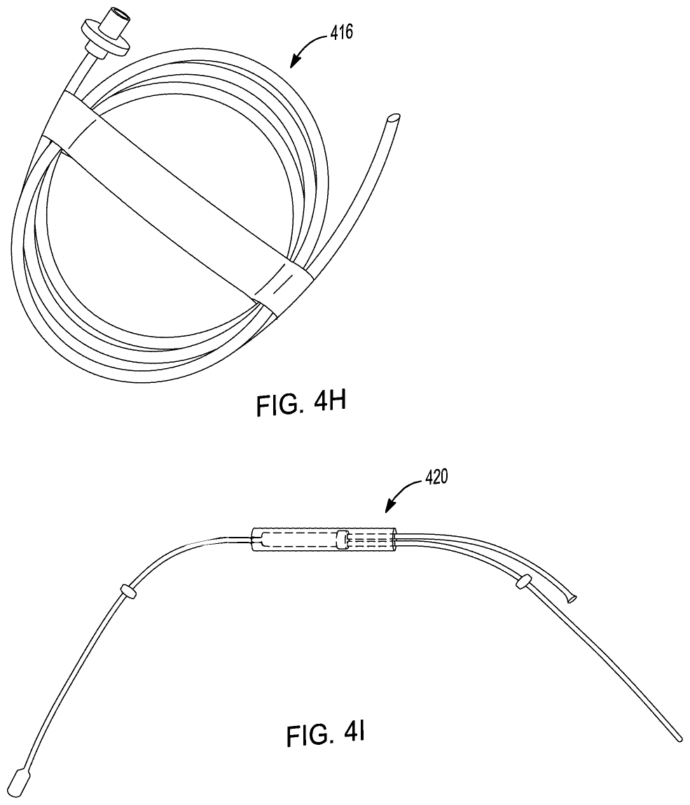

Turning to FIGS. 2A and 2B, an embodiment of a cell expansion system 200 with a premounted fluid conveyance assembly is shown in accordance with embodiments of the present disclosure. The CES 200 includes a cell expansion machine 202 that comprises a hatch or closable door 204 for engagement with a back portion 206 of the cell expansion machine 202. An interior space 208 within the cell expansion machine 202 includes features adapted for receiving and engaging a premounted fluid conveyance assembly 210. The premounted fluid conveyance assembly 210 is detachably-attachable to the cell expansion machine 202 to facilitate relatively quick exchange of a new or unused one of the premounted fluid conveyance assembly 210 at a cell expansion machine 202 for a used premounted fluid conveyance assembly 210 at the same cell expansion machine 202. A single cell expansion machine 202 may be operated to grow or expand a first set of cells using a first one of the premounted fluid conveyance assembly 210 and, thereafter, may be used to grow or expand a second set of cells using a second one of the premounted fluid conveyance assembly 210 without needing to be sanitized between interchanging the first one of the premounted fluid conveyance assembly 210 for the second one of the premounted fluid conveyance assembly 210. The premounted fluid conveyance assembly 210 includes the cell growth chamber 100 and an oxygenator or gas transfer module 212 (also see FIG. 4). Tubing guide slots are shown as 214 for receiving various media tubing connected to premounted fluid conveyance assembly 210, according to embodiments.

A front 262 of the CES 200 includes a user interface 264. The user interface 264 may include a display 268, such as a touch-screen, for example, to allow a user to enter data, retrieve data, enter test protocols, switch between views, view data, view alarms, etc. Additionally or alternatively, the user interface 264 may include one or more buttons or switches for entering information, controlling a display, or performing other functions. Input from the user interface 264 may be sent to the control system, as described below. The front 262 of the CES 200 may also include one or more lights or other visual signals for indicating alarms.

A holder 270, such as a bag holder, a disposables holder, etc., may extend from a top surface 272 of the CES 200. The holder 270 may include a vertical leg 274 and a horizontal leg 276 arranged as an L-shaped support 278, for example. A line support, or clamp, 280 may be attached to the vertical leg 274 of the L-shaped support 278 to support a portion of the disposable assembly, described below. For example, the line support 280 may support tubing of the disposable assembly relative to the vertical leg 274. A plurality of racks 282 may be supported by the horizontal leg 276 of the L-shaped support 278. Each of the plurality of racks 282 may support one or more bags of the disposables set, described below. For example, and as illustrated in FIG. 2C, each of the plurality of racks may include a rail system 284, a hook system 286, or both the rail system 284 and the hook system 286. The rail system 284 may include sidewalls 288 that extend parallel and define a channel 290 therebetween. The sidewalls 288 may be formed of metal, plastic, composite, or another suitable material. In an example embodiment, the sidewalls 288 may each include a secondary sidewall, or bumper, on an inside surface thereof which defines a neck 292, or narrow portion, of the channel 290. For example, the secondary sidewall 293 may be formed of an elastic or flexible material, such as rubber, or the like. In an alternative embodiment, the secondary sidewall may be formed of an inelastic material, such as metal, plastic, composite or another suitable material. For example, the neck 292 may be a narrower portion of the channel 290 such that the top of the bag or disposable is retained in the channel 290 by the neck 292. The rail system 284 may work to spread a load of the bag supported therein and reduce tearing, stretching, or ripping of the bag material. The rail system 284 is compatible with different bag types and sizes. The bags hung by the rail system 284 may include rods accepted in the channel 290 to retain the bags therein, as described below. The rod in the bag may have a diameter that is larger than a width of the neck 292 defined by the secondary sidewalls 293.

A bottom surface of the secondary sidewalls 293 may define slots 294 therein. The slots 294 may be positioned on a side of the secondary sidewalls 293 adjacent the sidewall 288. The slots 294 may be arranged to receive one or more hooks 296 of the hook system 286 therein. For example, slots 294 in a single secondary sidewall 293 of each rail system 284 may each receive a hook 296 of the hook system 286, while the slots 294 in the other secondary sidewall 293 of the rail system 284 may be left open. The hook 296 may be a U-shaped, or J-shaped hook and may be configured to secure a bag at an aperture in the bag.

The cell expansion system 200 and/or cell expansion machine 202 may include a barcode scanner configured to scan a barcode on a disposable, media, product, etc. to be used with the cell expansion system 200 and/or cell expansion machine 202. The barcode scanner is configured to collect data from the barcode that is scanned and transfer the data to the computing system 2400 described herein. The barcode scanner may be hard-wire attached or wireless. The barcode scanner may be a handheld device or fixed within the user interface or on the housing of the cell expansion machine 202. The barcode scanner is compatible to read customer bar codes, generic bar codes, supplier bar codes, or any other bar codes on or off the market.

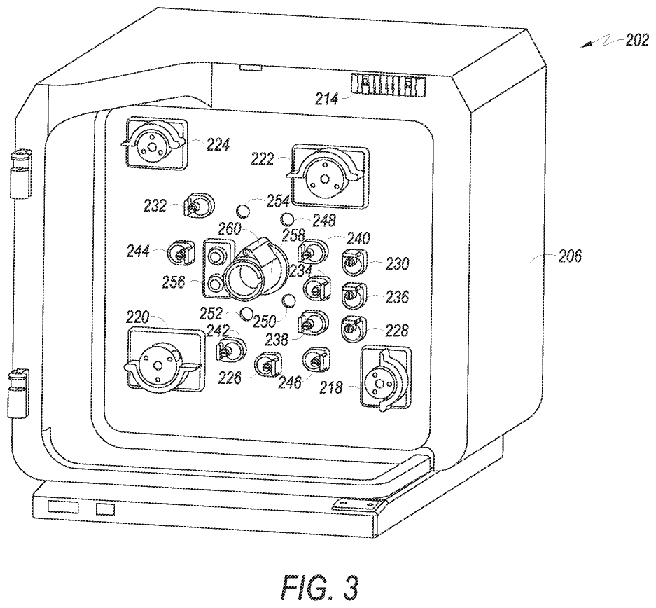

Next, FIG. 3 illustrates the back portion 206 of cell expansion machine 202 prior to detachably-attaching a premounted fluid conveyance assembly 210 (FIG. 2B), in accordance with embodiments of the present disclosure. The closable door 204 (shown in FIGS. 2A and 2B) is omitted from FIG. 3. The back portion 206 of the cell expansion machine 202 includes a number of different structures for working in combination with elements of a premounted fluid conveyance assembly 210. More particularly, the back portion 206 of the cell expansion machine 202 includes a plurality of peristaltic pumps for cooperating with pump loops on the premounted fluid conveyance assembly 210, including the IC circulation pump 218, the EC circulation pump 220, the IC inlet pump 222, and the EC inlet pump 224. In addition, the back portion 206 of the cell expansion machine 202 includes a plurality of valves, including the IC circulation valve 226, the reagent valve 228, the IC media valve 230, the air removal valve 232, the cell inlet valve 234, the wash valve 236, the distribution valve 238, the EC media valve 240, the IC waste or outlet valve 242, the EC waste valve 244, and the harvest valve 246. Several sensors are also associated with the back portion 206 of the cell expansion machine 202, including the IC outlet pressure sensor 248, the combination IC inlet pressure and temperature sensors 250, the combination EC inlet pressure and temperature sensors 252, and the EC outlet pressure sensor 254. Also shown is an optical sensor 256 for an air removal chamber, according to an embodiment.

In accordance with embodiments, a shaft or rocker control 258 for rotating the cell growth chamber 100 is shown. Shaft fitting 260 associated with the shaft or rocker control 258 allows for proper alignment of a shaft access aperture, see e.g., 424 (FIG. 4A) of a tubing-organizer, see e.g., 300 (FIG. 4) of a premounted conveyance assembly 210 or 400 with the back portion 206 of the cell expansion machine 202. Rotation of shaft or rocker control 258 imparts rotational movement to shaft fitting 260 and cell growth chamber 100. Thus, when an operator or user of the CES 200 attaches a new or unused premounted fluid conveyance assembly 400 (FIG. 4A) to the cell expansion machine 202, the alignment is a relatively simple matter of properly orienting the shaft access aperture 424 (FIG. 4A) of the premounted fluid conveyance assembly 210 or 400 with the shaft fitting 260.

Turning to FIG. 4A, a perspective view of a detachably-attachable premounted fluid conveyance assembly 400 is shown. The premounted fluid conveyance assembly 400 may be detachably-attachable to the cell expansion machine 202 (FIGS. 2B and 3) to facilitate relatively quick exchange of a new or unused premounted fluid conveyance assembly 400 at a cell expansion machine 202 for a used premounted fluid conveyance assembly 400 at the same cell expansion machine 202. As shown in FIG. 4A, the cell growth chamber 100 may be attached to a bioreactor coupling that includes a shaft fitting 402. The shaft fitting 402 includes one or more shaft fastening mechanisms, such as a biased arm or spring member 404 for engaging a shaft, e.g., 258 (shown in FIG. 3), of the cell expansion machine 202.

According to embodiments, the premounted fluid conveyance assembly 400 includes tubing 408A, 408B, 408C, 408D, 408E, etc., and various tubing fittings to provide the fluid paths shown in FIGS. 5A, 5B, 5C, and 6, as discussed below. Pump loops 406A, 406B, and 406C may also be provided for the pump(s). In embodiments, although the various media may be provided at the site where the cell expansion machine 202 is located, the premounted fluid conveyance assembly 400 may include sufficient tubing length to extend to the exterior of the cell expansion machine 202 and to enable welded connections to tubing associated with media bag(s) or container(s), according to embodiments.

Referring to FIGS. 4B-4H, alternative views of assembly and parts of the premounted fluid conveyance assembly 400 are illustrated. In example embodiments, the premounted fluid conveyance assembly 400 may include a media bag 410 (FIG. 4D), a waste bag 412 (FIG. 4E), a pressure pod 414 (FIGS. 4F-G), a sampling coil 416 (FIG. 4H), a cell input bag, an in-line filter 420 (FIG. 4I), additional tubing, additional filters, or a combination of these. For example, a premounted fluid conveyance assembly 400A in FIG. 4B may include the cell growth chamber 100B, while a premounted fluid conveyance assembly 400B in FIG. 4C may include the cell growth chamber 100A. Aside from a change in size of the cell growth chamber 100A, 100B, the remaining parts of the premounted fluid conveyance assemblies 400A and 400B may be the same. This allows for efficient manufacture of the premounted fluid conveyance assembly 400.

As illustrated in FIGS. 4D and 4E, the premounted fluid conveyance assembly 400 may include one or more media bags 410 and one or more waste bags 412. In some embodiments, the media bag(s) 410 may be the same as the waste bag(s) 412. Keeping the media bag(s) 410 the same as the waste bag(s) 412 allows for manufacturing efficiency and lower cost. As shown in FIGS. 4D and 4E, the media bag 410 and the waste bag 412 are formed of a sheet of material to define a material enclosure 422 and a hanger 425. For example, the sheet of material, the media bag 410, and the waste bag 412 may be formed of tri-layer EVA (ethylene-vinyl acetate), which is less breathable than PVC, to keep pH more stable so media is “happier” and the non-breathable material prolongs the life of the media. For example, the sheet of material may be welded, or melted, to form the material enclosure 422 and the hanger 425. The material enclosure 422 may be a pocket within the media bag 410 or the waste bag 412. The material enclosure 422 may be sized to hold a specific amount of fluid (for example, media). For example, the material enclosure 422 may be sized to hold 5 L of fluid. Alternatively, the material enclosure 422 may be sized to hold any amount of fluid appropriate for the desired function.

The hanger 425 may include apertures for receiving the hook(s) 296, as discussed with respect to FIG. 2C. The hanger 425 may include a rod 426 secured within a longitudinal aperture or channel 428 of the media bag 410 or the waste bag 412. The rod 426 may be a cylindrical-shaped or extruded polygonal-shaped rod that engages with the rail system 284, as discussed with respect to FIG. 2C. For example, the rod 426, when the bag is inserted within the channel 290 between sidewalls 288, rests on top of the secondary sidewalls 293 and has a diameter or width larger than the neck 292, such that the hanger 425 cannot slip back through the neck 292 and the media bag 410 or the waste bag 412 is suspended in the rail system 284. Additionally or alternatively, the media bag(s) 410 may be different materials, sizes, or configurations from the waste bag(s) 412.

A pair of ports 430 may be disposed on a section of the material enclosure 422 opposite the hanger 425. The pair of ports 430 may include an inlet port 430A and an outlet port 430B. The inlet port 430A may be engaged with a tube or fluid flow into the material enclosure 422. The outlet port 430B may be engaged with a tube or fluid flow out of the material enclosure 422. The pair of ports 430, or dual port design of the media bag 410 or the waste bag 412 allows for a number of media bags 410 or a number of waste bags 412 to be connected, or daisy-chain connected, together, as described below.

Referring to FIGS. 4F, and 4G, the pressure pod 414 may be disposed on a housing of the premounted fluid conveyance assembly 400. The pressure pod 414 may be configured to detect fluid pressure through a fluid line, tubing, or along a fluid flow path. The pressure pod 414 may include a single-outlet housing 433, a diaphragm 432, and a retaining ring 434. The single-outlet housing 433 may include a tubular inlet port 436 and a blocked port 438. The inlet port 436 may be a tubular port configured to receive fluid into the single-outlet housing 433. The blocked port 438 may similar to the tubular inlet port 436 but may be blocked with a plug 440 to prevent fluid flow therethrough. The plug 440 may be formed of plastic or an elastomer. The single-outlet housing 433 may include a body 442 formed monolithically with the inlet port 436 and the blocked port 438. The body 442 may define an interior space 444, for example, a cylindrical interior space 444 therein. The diaphragm 432 may be a circular planar diaphragm housed within the interior space 444 in the body 442. For example, the diaphragm 432 may be an elastic material that flexes with the pressure of fluid. The retaining ring 434 may be disposed at an end of the body 442 of the single-outlet housing 433 opposite the inlet port 436 and the blocked port 438 to secure the diaphragm 432 in the body 442. For example, the retaining ring 434 may be press-fit or threaded on an end of the body 442 opposite the inlet port 436 and the blocked port 438.

FIGS. 4H and 41 illustrate example tubing that may be included in the premounted fluid conveyance assembly 400. In at least one example embodiment, the sampling coil 416, shown in FIG. 4H, may be tubing configured to take a sample of fluid in one of the fluid flow paths. In at least one example embodiment, the in-line filter 420, shown in FIG. 4I, may include a filter positioned within tubing to filter materials from the fluid traveling through the fluid flow path.

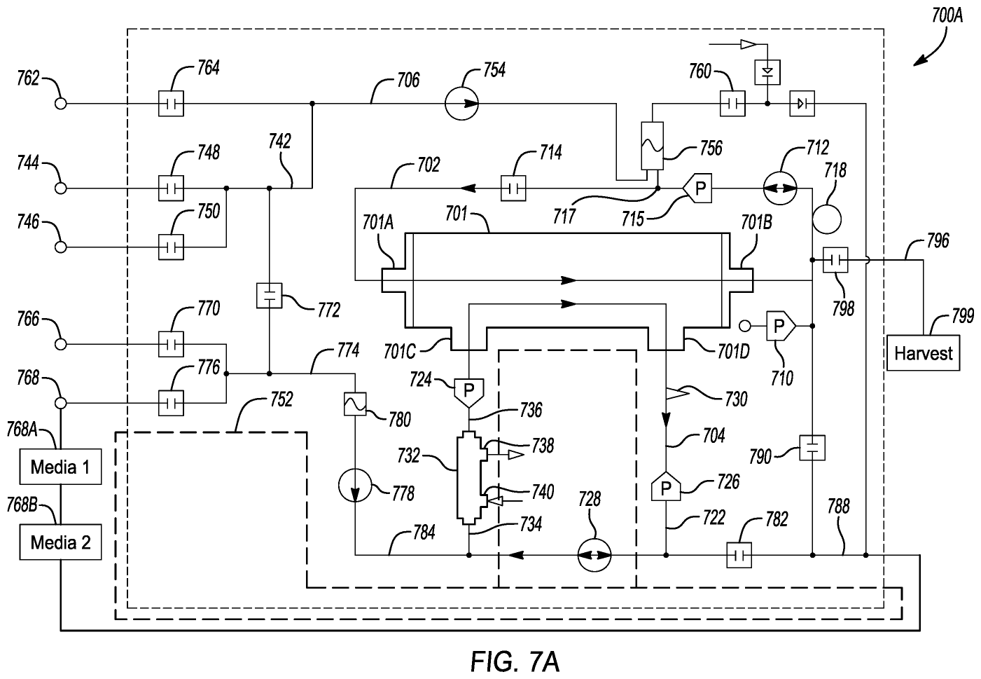

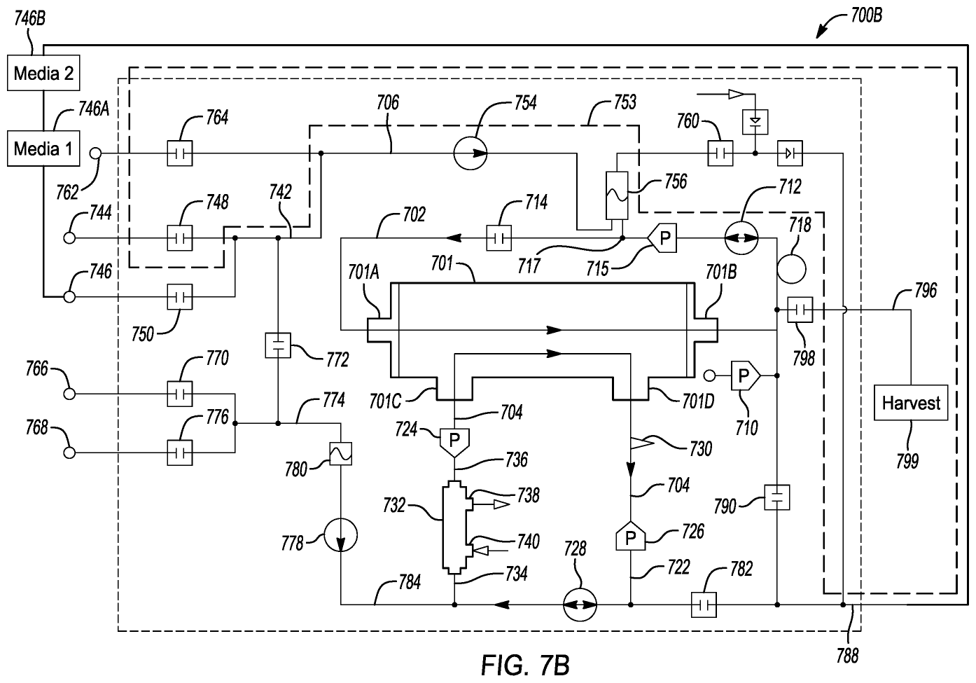

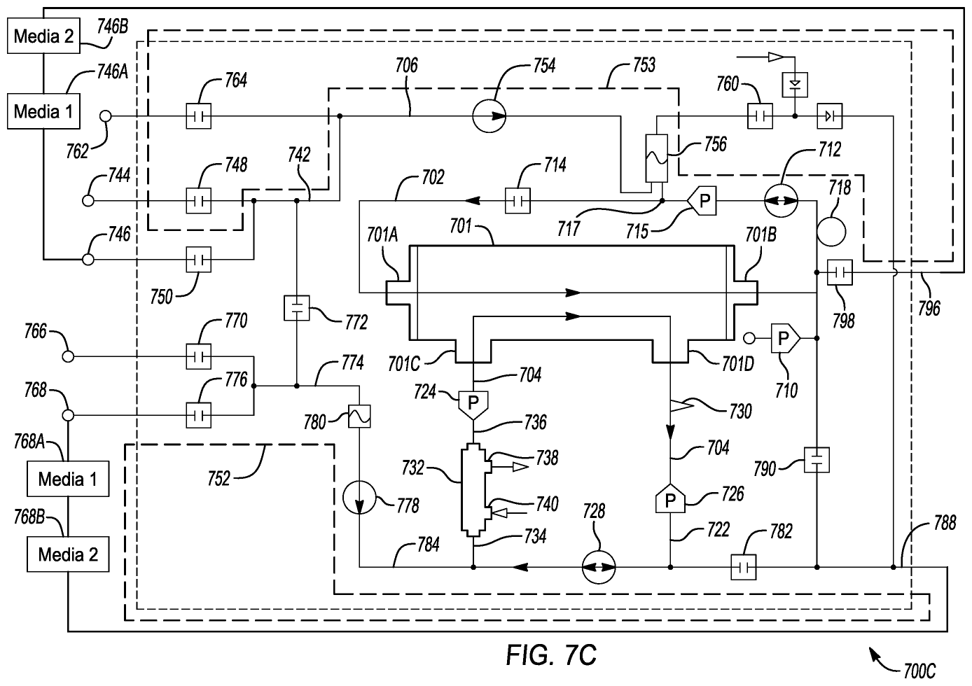

Next, FIGS. 5A, 5B, and 5C illustrate schematics of embodiments of a cell expansion system 500, FIG. 6 illustrates a schematic of another embodiment of a cell expansion system 600, and FIGS. 7A-7C illustrate a schematic of still another embodiment of a cell expansion system. In the embodiments shown in FIGS. 5A, 5B, 5C, and 6, and as described below, the cells are grown in the IC space. However, the disclosure is not limited to such examples and may in other embodiments provide for cells to be grown in the EC space.

As noted, FIGS. 5A, 5B, and 5C illustrate a CES 500. While FIGS. 5A, 5B, and 5C depict substantially similar structural components of CES 500, FIGS. 5A, 5B, and 5C illustrate possible operational configurations of fluid movement in a first fluid circulation path using the structural features of CES 500, in accordance with embodiments of the present disclosure. As shown, CES 500 includes first fluid circulation path 502 (also referred to as the “intracapillary loop” or “IC loop”) and second fluid circulation path 504 (also referred to as the “extracapillary loop” or “EC loop”), according to embodiments. First fluid flow path 506 may be fluidly associated with cell growth chamber 501 to form first fluid circulation path 502. Fluid flows into cell growth chamber 501 through IC inlet port 501A, through hollow fibers in cell growth chamber 501, and exits via IC outlet port 501B. Pressure gauge 510 measures the pressure of media leaving cell growth chamber or bioreactor 501. Media flows through IC circulation pump 512 which may be used to control the rate of media flow. IC circulation pump 512 may pump the fluid in a first direction or second direction opposite the first direction. IC outlet port 501B may be used as an inlet in the reverse direction. For example, in a first configuration, the IC circulation pump may pump the fluid in a positive direction, in which the fluid enters the IC inlet port 501A. In a second configuration, for example, the IC circulation pump may pump the fluid in a negative direction, in which the fluid enters the IC outlet port 501B, for example.

Media entering the IC loop may enter through valve 514. As those skilled in the art will appreciate, additional valves, pressure gauges, pressure/temperature sensors, ports, and/or other devices may be placed at various locations to isolate and/or measure characteristics of the media along portions of the fluid paths. Accordingly, it is to be understood that the schematic shown represents one possible configuration for various elements of the CES 500, and modifications to the schematic shown are within the scope of the one or more present embodiments.

With regard to the IC loop 502, samples of media may be obtained from sample port 516 or sample coil 518 during operation. Pressure/temperature gauge 520 disposed in first fluid circulation path 502 allows detection of media pressure and temperature during operation. Media then returns to IC inlet port 501A to complete fluid circulation path 502. Cells grown/expanded in cell growth chamber 501 may be flushed out of cell growth chamber 501 into cell harvest bag 599 through valve 598 or redistributed within the hollow fibers for further growth.

Fluid in second fluid circulation path 504 enters cell growth chamber 501 via EC inlet port 501C, and leaves cell growth chamber 501 via EC outlet port 501D. Media in the EC loop 504 may be in contact with the outside of the hollow fibers in the cell growth chamber 501, thereby allowing diffusion of small molecules into and out of the hollow fibers.

Pressure/temperature gauge 524 disposed in the second fluid circulation path 504 allows the pressure and temperature of media to be measured before the media enters the EC space of the cell growth chamber 501, according to an embodiment. Pressure gauge 526 allows the pressure of media in the second fluid circulation path 504 to be measured after it leaves the cell growth chamber 501. With regard to the EC loop, samples of media may be obtained from sample port 530 or a sample coil during operation.

In embodiments, after leaving EC outlet port 501D of cell growth chamber 501, fluid in second fluid circulation path 504 passes through EC circulation pump 528 to oxygenator or gas transfer module 532. EC circulation pump 528 may also pump the fluid in opposing directions. Second fluid flow path 522 may be fluidly associated with oxygenator or gas transfer module 532 via oxygenator inlet port 534 and oxygenator outlet port 536. In operation, fluid media flows into oxygenator or gas transfer module 532 via oxygenator inlet port 534, and exits oxygenator or gas transfer module 532 via oxygenator outlet port 536. Oxygenator or gas transfer module 532 adds oxygen to, and removes bubbles from, media in the CES 500, for example. In various embodiments, media in second fluid circulation path 504 may be in equilibrium with gas entering oxygenator or gas transfer module 532. The oxygenator or gas transfer module 532 may be any appropriately sized oxygenator or gas transfer device. Air or gas flows into oxygenator or gas transfer module 532 via filter 538 and out of oxygenator or gas transfer module 532 through filter 540. Filters 538 and 540 reduce or prevent contamination of oxygenator or gas transfer module 532 and associated media. Air or gas purged from the CES 500 during portions of a priming sequence may vent to the atmosphere via the oxygenator or gas transfer module 532.

In accordance with at least one embodiment, media, including cells (from bag 562), and fluid media from bag 546 may be introduced to first fluid circulation path 502 via first fluid flow path 506. Fluid container 562 (e.g., Cell Inlet Bag or Saline Priming Fluid for priming air out of the system) may be fluidly associated with the first fluid flow path 506 and the first fluid circulation path 502 via valve 564.

Fluid containers, or media bags, 544 (e.g., Reagent) and 546 (e.g., IC Media) may be fluidly associated with either first fluid inlet path 542 via valves 548 and 550, respectively, or second fluid inlet path 574 via valves 570 and 576. First and second sterile sealable input priming paths 508 and 509 are also provided. An air removal chamber (ARC) 556 may be fluidly associated with first circulation path 502. The air removal chamber 556 may include one or more ultrasonic sensors including an upper sensor and lower sensor to detect air, a lack of fluid, and/or a gas/fluid interface, e.g., an air/fluid interface, at certain measuring positions within the air removal chamber 556. For example, ultrasonic sensors may be used near the bottom and/or near the top of the air removal chamber 556 to detect air, fluid, and/or an air/fluid interface at these locations. Embodiments provide for the use of numerous other types of sensors without departing from the spirit and scope of the present disclosure. For example, optical sensors may be used in accordance with embodiments of the present disclosure. Air or gas purged from the CES 500 during portions of the priming sequence or other protocols may vent to the atmosphere out air valve 560 via line 558 that may be fluidly associated with air removal chamber 556.

EC media (e.g., from bag 568) or wash solution (e.g., from bag 566) may be added to either the first or second fluid flow paths. Fluid container 566 may be fluidly associated with valve 570 that may be fluidly associated with first fluid circulation path 502 via distribution valve 572 and first fluid inlet path 542. Alternatively, fluid container 566 may be fluidly associated with second fluid circulation path 504 via second fluid inlet path 574 and EC inlet path 584 by opening valve 570 and closing distribution valve 572. Likewise, fluid container 568 may be fluidly associated with valve 576 that may be fluidly associated with first fluid circulation path 502 via first fluid inlet path 542 and distribution valve 572. Alternatively, fluid container 568 may be fluidly associated with second fluid inlet path 574 by opening valve 576 and closing distribution valve 572.

An optional heat exchanger 552 may be provided for media reagent or wash solution introduction.

In the IC loop, fluid may be initially advanced by the IC inlet pump 554. In the EC loop, fluid may be initially advanced by the EC inlet pump 578. An air detector 580, such as an ultrasonic sensor, may also be associated with the EC inlet path 584.

In at least one embodiment, first and second fluid circulation paths 502 and 504 are connected to waste line 588. When valve 590 is opened, IC media may flow through waste line 588 and to waste or outlet bag 586. Likewise, when valve 582 is opened, EC media may flow through waste line 588 to waste or outlet bag 586.

In embodiments, cells may be harvested via cell harvest path 596. Here, cells from cell growth chamber 501 may be harvested by pumping the IC media containing the cells through cell harvest path 596 and valve 598 to cell harvest bag 599.

Various components of the CES 500 may be contained or housed within a machine or housing, such as cell expansion machine 202 (FIGS. 2 and 3), wherein the machine maintains cells and media, for example, at a predetermined temperature.

In the configuration depicted for CES 500 in FIG. 5A, fluid media in first fluid circulation path 502 and second fluid circulation path 504 flows through cell growth chamber 501 in the same direction (a co-current configuration), in an embodiment. The CES 500 may also be configured to flow in a counter-current conformation (not shown), in another embodiment. In the configuration shown in FIG. 5A, fluid in first fluid circulation path 502 enters the bioreactor 501 at IC inlet port 501A and exits the bioreactor 501 at IC outlet port 501B. In the configurations depicted in FIGS. 5B and 5C, fluid media in first circulation path 502 may flow in opposite or opposing directions from connection 517 such that fluid may enter IC inlet port, a first port, 501A on one end of the bioreactor, and fluid may enter IC outlet port, a second port, 501B on the opposing end of the bioreactor to retain cells in the bioreactor itself, according to embodiments. The first fluid flow path may be fluidly associated with the first fluid circulation path through connection 517. In embodiments, connection 517 may be a point or location from which the fluid may flow in opposite directions, for example, based on the direction of the IC inlet pump and the direction of the IC circulation pump. In an embodiment, connection 517 may be a T-fitting or T-coupling. In another embodiment, connection 517 may be a Y-fitting or Y-coupling. Connection 517 may be any type of fitting, coupling, fusion, pathway, tubing, etc., allowing the first fluid flow path to be fluidly associated with the first circulation path. It is to be understood that the schematics and operational configurations shown in FIGS. 5A, 5B, and 5C represent possible configurations for various elements of the cell expansion system, and modifications to the schematics and operational configurations shown are within the scope of the one or more present embodiments.

Turning to FIG. 6, a schematic of another embodiment of a cell expansion system 600 is shown. CES 600 includes a first fluid circulation path 602 (also referred to as the “intracapillary loop” or “IC loop”) and second fluid circulation path 604 (also referred to as the “extracapillary loop” or “EC loop”). First fluid flow path 606 may be fluidly associated with cell growth chamber 601 to form first fluid circulation path 602. Fluid flows into cell growth chamber 601 through IC inlet port 601A, through hollow fibers in cell growth chamber 601, and exits via IC outlet port 601B. Pressure sensor 610 measures the pressure of media leaving cell growth chamber 601. In addition to pressure, sensor 610 may, in embodiments, also be a temperature sensor that detects the media pressure and temperature during operation. Media flows through IC circulation pump 612 which may be used to control the rate of media flow. IC circulation pump 612 may pump the fluid in a first direction or second direction opposite the first direction. Exit port 601B may be used as an inlet in the reverse direction. Media entering the IC loop may enter through valve 614. As those skilled in the art will appreciate, additional valves, pressure gauges, pressure/temperature sensors, ports, and/or other devices may be placed at various locations to isolate and/or measure characteristics of the media along portions of the fluid paths. Accordingly, it is to be understood that the schematic shown represents one possible configuration for various elements of the CES 600, and modifications to the schematic shown are within the scope of the one or more present embodiments.

With regard to the IC loop, samples of media may be obtained from sample coil 618 during operation. Media then returns to IC inlet port 601A to complete fluid circulation path 602. Cells grown/expanded in cell growth chamber 601 may be flushed out of cell growth chamber 601 into cell harvest bag 699 through valve 698 and cell harvest path 697. Alternatively, when valve 698 is closed, the cells may be redistributed within chamber 601 for further growth.

Fluid in second fluid circulation path 604 enters cell growth chamber 601 via EC inlet port 601C and leaves cell growth chamber 601 via EC outlet port 601D. Media in the EC loop may be in contact with the outside of the hollow fibers in the cell growth chamber 601, thereby allowing diffusion of small molecules into and out of the hollow fibers that may be within chamber 601, according to an embodiment.

Pressure/temperature sensor 624 disposed in the second fluid circulation path 604 allows the pressure and temperature of media to be measured before the media enters the EC space of the cell growth chamber 601. Sensor 626 allows the pressure and/or temperature of media in the second fluid circulation path 604 to be measured after it leaves the cell growth chamber 601. With regard to the EC loop, samples of media may be obtained from sample port 630 or a sample coil during operation.

After leaving EC outlet port 601D of cell growth chamber 601, fluid in second fluid circulation path 604 passes through EC circulation pump 628 to oxygenator or gas transfer module 632. EC circulation pump 628 may also pump the fluid in opposing directions, according to embodiments. Second fluid flow path 622 may be fluidly associated with oxygenator or gas transfer module 632 via an inlet port 632A and an outlet port 632B of oxygenator or gas transfer module 632. In operation, fluid media flows into oxygenator or gas transfer module 632 via inlet port 632A, and exits oxygenator or gas transfer module 632 via outlet port 632B. Oxygenator or gas transfer module 632 adds oxygen to, and removes bubbles from, media in the CES 600, for example. In various embodiments, media in second fluid circulation path 604 may be in equilibrium with gas entering oxygenator or gas transfer module 632. The oxygenator or gas transfer module 632 may be any appropriately sized device useful for oxygenation or gas transfer. Air or gas flows into oxygenator or gas transfer module 632 via filter 638 and out of oxygenator or gas transfer module 632 through filter 640. Filters 638 and 640 reduce or prevent contamination of oxygenator or gas transfer module 632 and associated media. Air or gas purged from the CES 600 during portions of a priming sequence may vent to the atmosphere via the oxygenator or gas transfer module 632.

In the configuration depicted for CES 600, fluid media in first fluid circulation path 602 and second fluid circulation path 604 flows through cell growth chamber 601 in the same direction (a co-current configuration). The CES 600 may also be configured to flow in a counter-current conformation, according to embodiments.

In accordance with at least one embodiment, media, including cells (from a source such as a cell container, e.g., a bag) may be attached at attachment point 662, and fluid media from a media source may be attached at attachment point 646. The cells and media may be introduced into first fluid circulation path 602 via first fluid flow path 606. Attachment point 662 may be fluidly associated with the first fluid flow path 606 via valve 664, and attachment point 646 may be fluidly associated with the first fluid flow path 606 via valve 650. A reagent source may be fluidly connected to point 644 and be associated with first fluid inlet path 642 via valve 648, or second fluid inlet path 674 via valves 648 and 672.

Air removal chamber (ARC) 656 may be fluidly associated with first circulation path 602. The air removal chamber 656 may include one or more sensors including an upper sensor and lower sensor to detect air, a lack of fluid, and/or a gas/fluid interface, e.g., an air/fluid interface, at certain measuring positions within the air removal chamber 656. For example, ultrasonic sensors may be used near the bottom and/or near the top of the air removal chamber 656 to detect air, fluid, and/or an air/fluid interface at these locations. Embodiments provide for the use of numerous other types of sensors without departing from the spirit and scope of the present disclosure. For example, optical sensors may be used in accordance with embodiments of the present disclosure. Air or gas purged from the CES 600 during portions of a priming sequence or other protocol(s) may vent to the atmosphere out air valve 660 via line 658 that may be fluidly associated with air removal chamber 656.

An EC media source may be attached to EC media attachment point 668, and a wash solution source may be attached to wash solution attachment point 666, to add EC media and/or wash solution to either the first or second fluid flow path. Attachment point 666 may be fluidly associated with valve 670 that may be fluidly associated with first fluid circulation path 602 via valve 672 and first fluid inlet path 642. Alternatively, attachment point 666 may be fluidly associated with second fluid circulation path 604 via second fluid inlet path 674 and second fluid flow path 684 by opening valve 670 and closing valve 672. Likewise, attachment point 668 may be fluidly associated with valve 676 that may be fluidly associated with first fluid circulation path 602 via first fluid inlet path 642 and valve 672. Alternatively, attachment point 668 may be fluidly associated with second fluid inlet path 674 by opening valve 676 and closing distribution valve 672.

In the IC loop, fluid may be initially advanced by the IC inlet pump 654. In the EC loop, fluid may be initially advanced by the EC inlet pump 678. An air detector 680, such as an ultrasonic sensor, may also be associated with the EC inlet path 684.

In at least one embodiment, first and second fluid circulation paths 602 and 604 are connected to waste line 688. When valve 690 is opened, IC media may flow through waste line 688 and to waste or outlet bag 686. Likewise, when valve 692 is opened, EC media may flow to waste or outlet bag 686.

After cells have been grown in cell growth chamber 601, they may be harvested via cell harvest path 697. Here, cells from cell growth chamber 601 may be harvested by pumping the IC media containing the cells through cell harvest path 697, with valve 698 open, into cell harvest bag 699.

Various components of the CES 600 may be contained or housed within a machine or housing, such as cell expansion machine 202 (FIGS. 2 and 3), wherein the machine maintains cells and media, for example, at a predetermined temperature. It is further noted that, in embodiments, components of CES 600 and CES 500 may be combined. In other embodiments, a CES may include fewer or additional components than those shown in CES 500 and/or CES 600 and still be within the scope of the present disclosure. An example of a cell expansion system that may incorporate features of the present disclosure is the Quantum® Cell Expansion System, manufactured by Terumo BCT, Inc. in Lakewood, Colorado.

It is to be understood that the schematic shown in FIG. 6 represents a possible configuration for various elements of the cell expansion system, and modifications to the schematic shown are within the scope of the one or more present embodiments.

Examples and further description of cell expansion systems are provided in U.S. Pat. No. 8,309,347 (“Cell Expansion System and Methods of Use,” issued on Nov. 13, 2012) and U.S. Pat. No. 9,057,045, filed on Dec. 15, 2010, (“Method of Loading and Distributing Cells in a Bioreactor of a Cell Expansion System,” issued on Jun. 16, 2015), which are hereby incorporated by reference herein in their entireties for all that they teach and for all purposes.

In the embodiments shown in FIGS. 7A-7C, and as described below, the cells are grown in the IC space. However, the disclosure is not limited to such examples and may in other embodiments provide for cells to be grown in the EC space.

As noted, FIGS. 7A-7C illustrate a CES 700. While FIGS. 7A-7C depict substantially similar structural components of CES 700, FIGS. 7A-7C illustrate possible operational configurations of fluid movement in a first fluid circulation path using the structural features of CES 700, in accordance with embodiments of the present disclosure. As shown, CES 700 includes a first fluid circulation path 702 (also referred to as the “intracapillary loop” or “IC loop”) and a second fluid circulation path 704 (also referred to as the “extracapillary loop” or “EC loop”), according to embodiments. A first fluid flow path 706 may be fluidly associated with a cell growth chamber 701 to form the first fluid circulation path 702. Fluid is configured to flow into the cell growth chamber 701 through an IC inlet port 701A, through hollow fibers in cell growth chamber 701, and exit via an IC outlet port 701B. A pressure gauge 710 measures the pressure of media leaving the cell growth chamber 701 or the bioreactor. Media flows through an IC circulation pump 712, which may be used to control the rate of media flow. The IC circulation pump 712 may pump the fluid in a first direction or a second direction opposite the first direction. In at least one example embodiment, the IC outlet port 701B may be used as an inlet in the reverse direction. For example, in a first configuration, the IC circulation pump 712 may pump the fluid in a positive direction, in which the fluid enters the IC inlet port 701A. In a second configuration, for example, the IC circulation pump 712 may pump the fluid in a negative direction, in which the fluid enters the IC outlet port 701B.

Fluid in the IC loop, or the first fluid circulation path 702, may pass through an IC circulation valve 714. As those skilled in the art will appreciate, additional valves, pressure gauges, pressure/temperature sensors, ports, and/or other devices may be placed at various locations to isolate and/or measure characteristics of the media along portions of the fluid paths. For example, the first fluid circulation path 702 may include an IC inlet pressure sensor 715. The IC inlet pressure sensor 715 may be positioned between the IC circulation pump 712 and the IC circulation valves 714 in some example embodiments. Accordingly, it is to be understood that the schematic shown represents one possible configuration for various elements of the CES 700, and modifications to the schematic shown are within the scope of the one or more present example embodiments.

With regard to the first fluid circulation path 702, samples of media may be obtained from a sample port or a sample coil 718 during operation. A pressure/temperature gauge 720 disposed in the first fluid circulation path 702 allows detection of media pressure and temperature during operation. Media then returns to IC inlet port 701A to complete the first fluid circulation path 702. Cells grown and/or expanded in the cell growth chamber 701 may be flushed out of the cell growth chamber 701 into a harvest bag 799 through a valve 798 or redistributed within the hollow fibers for further growth.

Fluid in the second fluid circulation path 704 enters the cell growth chamber 701 via an EC inlet port 701C, and leaves the cell growth chamber 701 via an EC outlet port 701D. Media in the second fluid circulation path 704 may be in contact with the outside of the hollow fibers in the cell growth chamber 701, thereby allowing diffusion of small molecules into and out of the hollow fibers.

In at least one example embodiment, a pressure/temperature gauge 724 disposed in the second fluid circulation path 704 allows the pressure and/or temperature of media to be measured before the media enters the EC space of the cell growth chamber 701. A pressure gauge 726 allows the pressure of media in the second fluid circulation path 704 to be measured after it leaves the cell growth chamber 701. With regard to the EC loop, samples of media may be obtained from a sample port 730 or a sample coil during operation.

In at least one example embodiment, after leaving the EC outlet port 701D of the cell growth chamber 701, fluid in the second fluid circulation path 704 passes through an EC circulation pump 728 and to an oxygenator or gas transfer module 732. The EC circulation pump 728 may also pump the fluid in opposing directions. A second fluid flow path 722 may be fluidly associated with the oxygenator or gas transfer module 732 via an oxygenator inlet port 734 and an oxygenator outlet port 736. In operation, fluid media flows into the oxygenator or gas transfer module 732 via the oxygenator inlet port 734, and exits the oxygenator or gas transfer module 732 via the oxygenator outlet port 736. The oxygenator or gas transfer module 732 adds oxygen to, and removes bubbles from, media in the CES 700, for example. In various example embodiments, media in the second fluid circulation path 704 may be in equilibrium with gas entering the oxygenator or gas transfer module 732. The oxygenator or gas transfer module 732 may be any appropriately sized oxygenator or gas transfer device. Air or gas flows into the oxygenator or gas transfer module 732 via a filter 738 and out of the oxygenator or gas transfer device 732 through a filter 740. The filters 738 and 740 reduce or prevent contamination of the oxygenator or gas transfer module 732 and associated media. Air or gas purged from the CES 700 during portions of a priming sequence may vent to the atmosphere via the oxygenator or gas transfer module 732.

In accordance with at least one embodiment, media, including cells from bag 762 and fluid media from bag 746, may be introduced to the first fluid circulation path 702 via the first fluid flow path 706. The fluid container 762 (e.g., Cell Inlet Bag or Saline Priming Fluid for priming air out of the system) may be fluidly associated with the first fluid flow path 706 and the first fluid circulation path 702 via a valve 764.

Fluid containers, or media bags, 744 (e.g., Reagent) and 746 (e.g., IC Media) may be fluidly associated with either a first fluid inlet path 742 via valves 748 and 750, respectively, or a second fluid inlet path 774 via valves 770 and 776. An air removal chamber (ARC) 756 may be fluidly associated with first circulation path 702. The air removal chamber 756 may include one or more ultrasonic sensors including an upper sensor and a lower sensor to detect air, a lack of fluid, and/or a gas/fluid interface, e.g., an air/fluid interface, at certain measuring positions within the air removal chamber 756. For example, ultrasonic sensors may be used near the bottom and/or near the top of the air removal chamber 756 to detect air, fluid, and/or an air/fluid interface at these locations. Example embodiments provide for the use of numerous other types of sensors without departing from the spirit and scope of the present disclosure. For example, optical sensors may be used in accordance with embodiments of the present disclosure. Air or gas purged from the CES 700 during portions of the priming sequence or other protocols may vent to the atmosphere out an air valve 760 that may be fluidly associated with the air removal chamber 756.

The EC media (e.g., from the bag 768) and/or the wash solution (e.g., from the bag or the fluid container 766) may be added to the first fluid inlet path 742 and/or the second fluid flow path 722. The fluid container 766 may be fluidly associated with valve 770 that may be fluidly associated with first fluid circulation path 702 via distribution valve 772 and first fluid inlet path 742. Alternatively, the fluid container 766 may be fluidly associated with the second fluid circulation path 704 via the second fluid inlet path 774 and the EC inlet path 784 by opening the valve 770 and closing the distribution valve 772. Likewise, as shown in FIG. 7C, the fluid containers 768A and 768B may be fluidly associated with the valve 776 that may be fluidly associated with first fluid circulation path 702 via the first fluid inlet path 742 and the distribution valve 772. Alternatively, fluid containers 768A and 768B may be fluidly associated with the second fluid inlet path 774 by opening valve 776 and closing distribution valve 772.

The fluid containers 768A and 768B may be media containers for dispensing media into the first fluid inlet path 742 or the second fluid inlet path 774. In some example embodiments, the fluid containers 768A and 768B may be the same as the media bag 410 illustrated in FIG. 4D. The fluid containers 768A and 768B may be a portion of an EC circulation feed loop 752 when in communication with the second fluid inlet path 774 (or an IC circulation feed loop when in communication with the first fluid inlet path 742). In the EC circulation feed loop 752, the media is transferred from fluid container 768B to fluid container 768A through EC loop 704, through EC waste valve 782, and back to fluid container 768B. Using the EC circulation feed loop 752 avoids use of a pass-through profusion feeding where fresh media is continuously added to the system and collected in an outlet waste bag which is discarded when full. With pass-through profusion feeding, as you grow more cells, media has to be brought in at a higher rate to feed the cells. A user typically takes a reading for the glucose or lactate once per day and then increases the feed rate for the day, for example. The feed rate may not be correct for the whole day leading to wasted media.

Instead, in the CES 700 herein, the EC circulation feed loop 752 enlarges the volume of either the IC circulation loop or the EC circulation loop by connecting, or daisy-chain connecting, the fluid container 768A with the fluid container 768B (though the pair of ports previously discussed). With the CES 700, the cell growth chamber 701 is hand fed outside the CES 700. The outlet line 788 goes back to the fluid container 768B and the media is recirculated. The user may calculate how much lactate is produced to determine waste. A user does not need to change bags; they only set the feed rate and run the protocol. Thus, the CES 700 is more efficient and uses less overall media.

While two fluid containers 768A and 768B are illustrated and discussed, it is understood that more fluid containers 768N may be added to the system.

In an example protocol, gas is supplied to the system via a GTM, such as a GTM 732, using EC circulation. The gas is supplied externally to the CES 700 and each time the media passes through the GTM 732, gas is exchanged to the desired concentration. In this case, the fluid containers 768A and 768B serve as exchange vessels to the media returning to the cell growth chamber 701. For example, the attached T-Cell feeding protocol (CFA-CSS-CEdj-036-03) uses approximately 11 L of EC media and 3.4 L of IC media over the duration of the growth protocol. Using this new feed strategy, the user would attach 3.4 L of IC media to the IC media line and daisy chain 11 L (3 media bags) of EC media and attach it to the EC inlet line and the outlet line. Just as EC circulation rates need to increase as the cell population increases demand for oxygen, the EC inlet rate (effectively EC feed circulation) would increase.

Depending on the cell type and protocol dynamics, either the IC feed circulation loop or the EC feed circulation loop can be implemented. Alternatively, dual feed circulation loops can be used, as shown in FIG. 7C, for example.

This strategy allows a large number of possible feeding strategy implementations. This strategy also enhances the ease of use for the CES 700, less human interaction with the device is required as waste bags do not need to be emptied and new media bags are supplied to the machine at various intervals during cell growth. Additionally, feed rates do not have to be determined in many of these scenarios. Even using continuous perfusion feeding, the lactate and glucose concentrations can fluctuate significantly without the use of very lengthy custom tasks that change the flow rates in small increments.

Depending on desired use, the EC circulation feed loop 752 may be used in coating the fibers of the cell growth chamber 701 to circulate and push fluid through the membrane (on the IC side). Additionally, or alternatively, the EC circulation feed loop 752 may be used in counter flow technology to push fluid on opposite ends of cell growth chamber 701.

A passive coating model used to in CES systems may require a coating agent to be applied to the cell growth surface in order promote cell adherence and subsequent expansion of an adherent cell line such as human mesenchymal stromal cells (hMSCs). Example coating agents are human fibronectin (hFN) or cryoprecipitate (CPPT). Bioreactor coating protocols for the passive coating model load the coating agent into the intracapillary side of the bioreactor and circulate the coating agent in the IC Circulation loop for a minimum of 16 hours. The passive coating model protocol requires at least two CES systems to immediately begin additional expansion of a population of cells harvested from a CES system (hMSCs cannot be stored in a non-cryopreserved state for up to 16 hours). A new protocol or method for coating the fibers of the cell growth chamber 701 results in a successful coating of the growth surface 10 minutes after loading the coating agent into the IC Circulation loop. This method (see Appendix B for protocol) utilizes positive ultra-filtration of the fluid (moving fluid from the IC side of the bioreactor to the EC side of the bioreactor) in order to decrease the time required for the proper chemical reaction between the coating agent and the growth surface of the bioreactor. The molecular barrier created by the specified construction of the hollow fibers in the bioreactor is such that the coating agent is not able to pass through the fiber wall along with the fluid it is suspended in. Moving the fluid using positive ultra-filtration results in “actively” promoting the coating agent to the surface of the hollow fiber. Allowing users to coat the hollow fiber bioreactor and load cells in the same day reduces errors, saves time, and reduces the length of cell expansions by one day. This also allows customers with access to only a single CES device, such as a Quantum®/Quantum Flex® device, to harvest a cell population from that system and coat a new disposable for subsequent passage/expansion of that same cell population without the need for cryopreservation.

Using the 2-port bag, as described with reference to FIGS. 4D and 4E, the inlet line (typically the ‘Wash’ line) can be connected to one port of the bag while the ‘Waste’ line can be connected to the other. This allows for recirculation of the protein vehicle fluid (typically phosphate buffered saline (PBS)) containing the coating reagent through the system for as long as the user would like. Using high IC inlet rates to drive contact between the coating solution and the HFB membrane via ultrafiltration (UF) drives adsorption of the coating reagent and reduces the time required to coat the hollow fiber bioreactor when compared to the passive model of coating. Moving IC fluid across the HFB membrane also enhances deposition of coating reagent onto the IC side of the HFB membrane. For example, because the coating solution has a higher molecular weight and cannot cross the HFB membrane, it is deposited onto IC side of the HFB membrane.

An optional heat exchanger may be provided for media reagent or wash solution introduction. In some embodiments, the optional heat exchanger may be provided in the first fluid inlet path 742 and/or the second fluid inlet path 774.

One or more differential pressure sensor(s) may be included for live pressure monitoring and alarming providing more efficient and accurate measurement as opposed to manual measurement. Pressure sensors facilitate detection of reduced or stopped flow, pressure sensor triggers an alarm to alert the user. For example, the alarm may be a remote alarm, an audio alarm on the device, a visual alarm on the device, or a combination thereof. For example, the remote alarm may be an email, a text message, or other digital alert sent to the user.

One or more gas regulator(s) may be included for gas management. For example, the gas regulator(s) may be internal to the CES 700 such that it is not user facing, preventing errors. The gas regulator(s) may be less sensitive to movement, providing fine control. Upon detection of errors in gas management, the gas regulator(s) may trigger an alarm to alert the user. For example, the alarm may be a remote alarm, an audio alarm on the device, a visual alarm on the device, or a combination thereof. For example, the remote alarm may be an email, a text message, or other digital alert sent to the user.

One or more temperature thermistors may be included for high sensitivity temperature detection. A 0.5-degree temperature change is critical in cell cultures. Thermistors have less degradation compared with resistance temperature detectors (RTDs) and therefore allow for more use before required service. Upon detection of a temperature change outside of a predetermined range or a temperature above or below a predetermined threshold, the thermistor may trigger an alarm to alert the user. For example, the alarm may be a remote alarm, an audio alarm on the device, a visual alarm on the device, or a combination thereof. For example, the remote alarm may be an email, a text message, or other digital alert sent to the user.

In the IC loop, fluid may be initially advanced by the IC inlet pump 754. In the EC loop, fluid may be initially advanced by the EC inlet pump 778. An air detector 780, such as an ultrasonic sensor, may also be associated with the EC inlet path 784. Upon detection of air by the air detector 780, the air detector may trigger an alarm to alert the user. For example, the alarm may be a remote alarm, an audio alarm on the device, a visual alarm on the device, or a combination thereof. For example, the remote alarm may be an email, a text message, or other digital alert sent to the user.

In at least one embodiment, first and second fluid circulation paths 702 and 704 are connected to outlet line 788. When valve 790 is opened, IC media may flow through outlet line 788 and return to fluid container 768B. Likewise, when valve 782 is opened, EC media may flow through outlet line 788 back to fluid container 768B.

In example embodiments, cells may be harvested via cell harvest path 796. Here, cells from cell growth chamber 701 may be harvested by pumping the IC media containing the cells through cell harvest path 796 and valve 798 to cell harvest bag 799.

Various components of the CES 700 may be contained or housed within a machine or housing, such as cell expansion machine 202 (FIGS. 2A, 2B, and 3), wherein the machine maintains cells and media, for example, at a predetermined temperature.

In the configuration depicted for CES 700 in FIG. 7A, fluid media in first fluid circulation path 702 and second fluid circulation path 704 flows through cell growth chamber 701 in the same direction (a co-current configuration), in an embodiment. The CES 700 may also be configured to flow in a counter-current conformation (not shown), in another embodiment. In the configuration shown in FIG. 7A, fluid in first fluid circulation path 702 enters the cell growth chamber 701 at IC inlet port 701A and exits the cell growth chamber 701 at IC outlet port 701B. In alternative configurations, fluid media in first circulation path 702 may flow in opposite or opposing directions from connection 717 such that fluid may enter IC inlet port, a first port, 701A on one end of the cell growth chamber 701, and fluid may enter IC outlet port, a second port, 701B on the opposing end of the cell growth chamber 701 to retain cells in the cell growth chamber itself, according to embodiments. The first fluid flow path may be fluidly associated with the first fluid circulation path through connection 717. In embodiments, connection 717 may be a point or location from which the fluid may flow in opposite directions, for example, based on the direction of the IC inlet pump and the direction of the IC circulation pump. In an embodiment, connection 717 may be a T-fitting or T-coupling. In another embodiment, connection 717 may be a Y-fitting or Y-coupling. Connection 717 may be any type of fitting, coupling, fusion, pathway, tubing, etc., allowing the first fluid flow path to be fluidly associated with the first circulation path. It is to be understood that the schematics and operational configurations shown in FIGS. 7A-7C represent possible configurations for various elements of the cell expansion system, and modifications to the schematics and operational configurations shown are within the scope of the one or more present embodiments.

Referring to FIG. 7B, a CES 700B is illustrated. CES 700B may be similar to CES 700A and may include the same or similar components as CES 700A where like reference numbers are used. CES 700B may include an IC circulation feed loop 753. IC circulation feed loop 753 may be similar to EC circulation feed loop 752, as previously described. IC circulation feed loop 753 may include multiple fluid containers, for example, fluid container 746A and fluid container 746B. Fluid container 746A and fluid container 746B may be the same as the media bag 410 illustrated in FIG. 4D.

Fluid containers 746A and 746B may be media containers for dispensing media into the first fluid flow path 706 through the IC media line 746. In the IC circulation feed loop 753, the media is transferred from fluid container 746B to fluid container 746A through IC loop 702, through the cell growth chamber 701, through IC waste valve 790, and back to fluid container 746B. Using the IC circulation feed loop 753 avoids use of a pass-through profusion feeding where fresh media is continuously added to the system and collected in an outlet waste bag which is discarded when full. With pass-through profusion feeding, as you grow more cells, media has to be brought in at a higher rate to feed the cells. A user typically takes a reading for the glucose or lactate once per day and then increases the feed rate for the day. The feed rate may not be correct for the whole day leading to wasted media.

Instead, in the CES 700 herein, the IC circulation feed loop 753 enlarges the volume of the IC circulation loop by connecting, or daisy-chain connecting, the fluid container 746A with the fluid container 746B (through the pair of ports previously described). With the CES 700, the cell growth chamber 701 is hand fed outside the CES 700. The outlet line 788 goes back to the fluid container 746B and the media is recirculated. The user may calculate how much lactate is produced to determine waste. A user does not need to change bags; they only set the feed rate and run the protocol. Thus, the CES 700 is more efficient and uses less overall media.

While two fluid containers 746A and 746B are illustrated and discussed, it is understood that more fluid containers 746N may be added to the system.

This strategy allows a large number of possible feeding strategy implementations. This strategy enhances the ease of use for the CES 700 as well, with less human interaction with the device as waste bags don't need to be emptied and new media bags supplied to the machine at various intervals during cell growth. Feed rates do not have to be determined in many of these scenarios either. Even using continuous perfusion feeding, the lactate and glucose concentrations can fluctuate significantly without the use of very lengthy custom tasks that change the flow rates in small increments.

Depending on desired use, the IC circulation feed loop 753 may be used in coating the fibers of the cell growth chamber 701 to circulate and push fluid through membrane (on IC side). Additionally, or alternatively, the IC circulation feed loop 753 may be used in counter flow technology to push fluid on opposite ends of cell growth chamber 701.

One or more differential pressure sensor(s) may be included for live pressure monitoring and alarming providing more efficient and accurate measurement as opposed to manual measurement. Pressure sensors facilitate detection of reduced or stopped flow, pressure sensor triggers an alarm to alert the user. For example, the alarm may be a remote alarm, an audio alarm on the device, a visual alarm on the device, or a combination thereof. For example, the remote alarm may be an email, a text message, or other digital alert sent to the user.

One or more gas regulator(s) may be included for gas management. For example, the gas regulator(s) may be internal to the CES 700 such that it is not user facing, preventing errors. The gas regulator(s) may be less sensitive to movement, providing fine control. Upon detection of errors in gas management, the gas regulator(s) may trigger an alarm to alert the user. For example, the alarm may be a remote alarm, an audio alarm on the device, a visual alarm on the device, or a combination thereof. For example, the remote alarm may be an email, a text message, or other digital alert sent to the user.

One or more temperature thermistors may be included for high sensitivity temperature detection. A 0.5-degree temperature change is critical in cell cultures. Thermistors have less degradation compared with resistance temperature detectors (RTDs) and therefore allow for more use before required service. Upon detection of a temperature change outside of a predetermined range or a temperature above or below a predetermined threshold, the thermistor may trigger an alarm to alert the user. For example, the alarm may be a remote alarm, an audio alarm on the device, a visual alarm on the device, or a combination thereof. For example, the remote alarm may be an email, a text message, or other digital alert sent to the user.

Now referring to FIG. 7C, a CES 700C having dual feed circulation loops is illustrated. CES 700C may be similar to CES 700A and CES 700B and may include the same or similar components as CES 700A and CES 700B where like reference numbers are used. CES 700C may include the EC circulation feed loop 752 from CES 700A and the IC circulation feed loop 753 from CES 700B. The IC circulation feed loop 753 may pass fluid from the fluid container 746B to the fluid container 746A, to the first fluid inlet path 742, to the cell growth chamber 701, to the harvest valve 798 and back to the fluid container 746B. The EC circulation feed loop 752 may pass fluid from the fluid container 768B to the fluid container 768A, to the EC media valve 776, to the second fluid inlet path 774, to the GTM 732, to the cell growth chamber 701, to the EC waste valve 782 and back to the fluid container 768B.

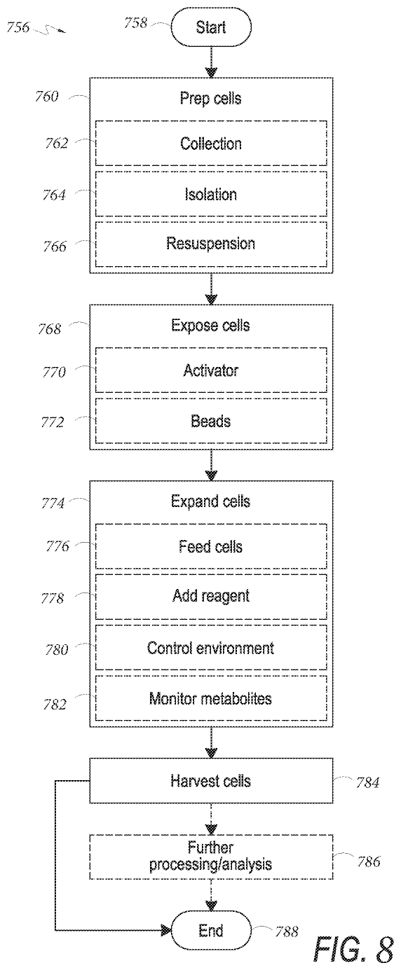

While various example embodiments of a cell expansion system and methods associated therewith have been described, FIG. 8 illustrates example operational steps 756 of a process for expanding non-adherent, or suspension, cells in a cell expansion system, such as CES 500, CES 600, or CES 700A-700C in accordance with embodiments of the present disclosure.

START operation 758 is initiated, and process 756 proceeds to preparation of cells 760. In embodiments, the preparation of cells 760 may involve a number of different and optional steps. For example, the cells may be collected 762. The collection of cells 762 may involve separating and collecting the cells from a donor. In some embodiments, an apheresis procedure may be performed to collect a volume of lymphocytes from the peripheral blood of a donor, e.g., leukapheresis. The volume of lymphocytes may include the target cell population to be expanded by process 756. In other embodiments, the cells may be collected from cord blood.

After collection 762, optionally, the cells may be isolated 764 as part of the preparation 760. The volume of cells collected at step 762 may include a number of different cell types including the cells that are targeted for expansion. Optional step 764 may be performed to isolate the target cells. As one example, the target cells may be T cells, e.g., regulated T cells. In one embodiment, the regulated T cells may be CD4+CD25+ T cells. The cells may be isolated using any suitable isolation technique. For example, the cells may be isolated using immunomagnetic separation where magnetic beads functionalized with antibodies are contacted with the cells collected at 762. The functionalized beads may preferentially attach to the target cell population. A magnetic field may then be used to retain the beads with the attached target cell population, while the other cells may be removed.

The cells may be optionally resuspended 766 after isolation 764. In embodiments, the cells may be resuspended in a media that includes a number of nutrients and/or reagents that aid in maintaining the viability of the cells. In embodiments, the media may include at least serum albumin and a reagent, such as a cytokine. The cytokine may in embodiments be a recombinant human IL-2 cytokine. The media may include the cytokine at a concentrate of 200 IU/ml, in one embodiment.

Following the preparation of the cells 760, process 756 proceeds to expose cells 768 in order to activate the cells to expand. The cells may optionally be exposed to an activator at 770 that is soluble. The activator, which may include antibody complexes, may be added to the media in which the cells are resuspended. In embodiments, the activator may be a human antibody CD3/CD28/CD2 cell activator complex. In some embodiments, the activator, may be included in the media used in the resuspension of the cells 766. Optionally, the cells may be exposed to beads at 772, which may have an activator on their surface. In embodiments, exposing the cells to the beads may involve adding a predetermined amount of beads to the resuspended cells. The beads may be added at different ratios with respect to the number of cells. For example, the beads may be added in a 1 bead:2 cell ratio. Other embodiments may provide for adding beads at different ratios, e.g., 1 bead:1 cell, 1 bead:3 cells, etc. The beads may have antibodies on their surface to activate the cells to expand. In embodiments, the beads may include antibodies CD3/CD28 on their surface.

Process 756 proceeds to expand cells 774. As part of cell expansion at 774, the cells may be loaded into a cell growth chamber, e.g., a hollow fiber membrane bioreactor, where the cells are expanded. The cells may be fed nutrients at 776 to promote their expansion. For example, media may be delivered into the cell growth chamber to provide the nutrients for expansion. For example, media may be provided through the IC circulation feed loop 753, the EC circulation feed loop 752, or a combination thereof, as discussed with respect to FIGS. 7A-7C. The expansion of the cells 774 may also include adding reagents at 778 periodically to the cell growth chamber to continue to promote their expansion. For example, in some embodiments, reagents (e.g., cytokines) may be added to the cell growth chamber to promote the expansion of the cells. In one embodiment, the reagent may be additional IL-2 cytokine, e.g., recombinant human IL-2 cytokine.

For example, media, reagents, or a combination thereof may be delivered to the cells from one or more connected fluid containers, as described with respect to FIGS. 7A-7C may circulate, and re-circulate, the media, reagents, or combination thereof through the IC circulation feed loop 753 or the EC circulation feed loop 752.

Also, as part of expanding the cells 774, the environment inside the cell growth chamber may be controlled at 780. For example, gasses may be delivered and exchanged continuously to provide a balance of for example, carbon dioxide and oxygen to the cells expanding in the cell growth chamber. Additionally, the temperature may be controlled to be within a range optimized for cell expansion. Expansion of cells 774 may also include monitoring metabolites at 782. For example, the lactate and glucose levels may be periodically monitored. The lactate and glucose levels may be monitored by one or more sensors. The one or more sensors may also be configured to measure oxygen levels. A rise or fall in the metabolites may prompt changes (e.g., additional feeding, additional reagent additions, additional gas exchange, etc.) to control the environment at 780 in the cell growth chamber.

As various parameters are monitored, data readings falling outside of predetermined thresholds may trigger an alarm to alert the user. For example, the data readings may be a temperature, a door position, a pressure, a flow rate, or a concentration. For example, the alarm may be a remote alarm, an audio alarm on the device, a visual alarm on the device, or a combination thereof. For example, the remote alarm may be an email, a text message, or other digital alert sent to the user.

Process 756 next proceeds to harvest the cells at 784. Further processing of the removed cells or other analysis may optionally be performed at step 786. For example, the cells may be characterized to determine cell phenotype(s). The further processing or other analysis at 786 may include performing flow cytometry, for example, to characterize cell phenotypes. Process 756 may then terminate at END operation 788. If it is not desired to perform further processing/analysis, process 756 terminates at END operation 788.

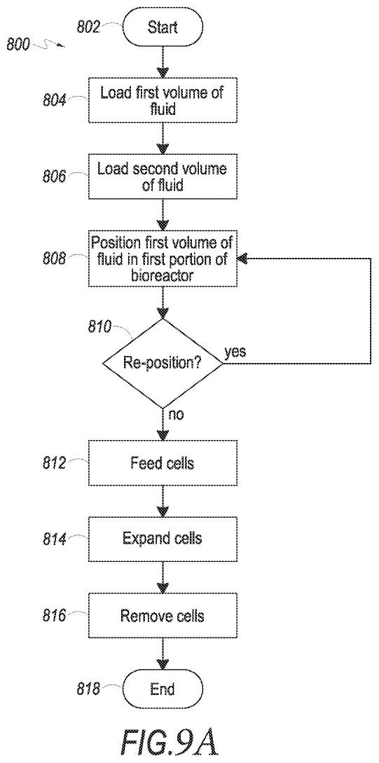

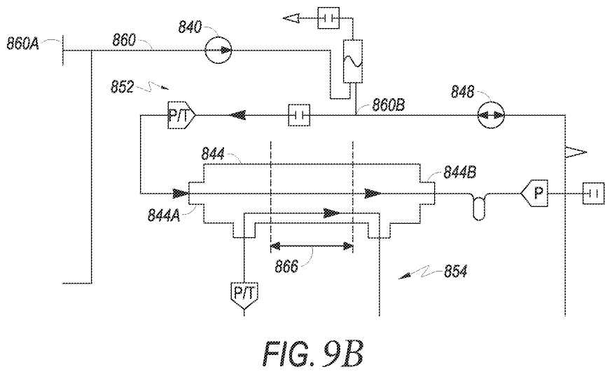

FIG. 9A illustrates operational steps of a process 800, which may be used to position cells or other material (e.g., proteins, nutrients, growth factors) into a cell growth chamber according to embodiments of the present disclosure. In embodiments, the process 800 may be implemented as part of a “load cells centrally without circulation” task. Start operation 802 is initiated and process 800 proceeds to step 804, where a first volume of fluid with cells may be loaded into a cell growth chamber of a cell expansion system. In embodiments, the cells may comprise non-adherent cells, such as one or more types of T cells. In one embodiment, the plurality of cells comprises Tregs. As may be appreciated, loading of the first volume of fluid with cells may be performed by components of a cell expansion system such as systems CES 500 (e.g., FIG. 5A), CES 600 (FIG. 6), and CES 700 (FIGS. 7A-7C), described above. FIG. 9B illustrates a portion of a cell expansion system that includes a first fluid inlet pump 840, a first fluid flow path 860, a first fluid circulation pump 848, a first fluid circulation path 852, a cell growth chamber 844, and a second fluid circulation path 854. The first fluid flow path 860 is fluidly associated with fluid circulation path 852 through connection 860B. Embodiments may provide for the first volume of fluid with the cells to be loaded at 804 through the first fluid flow path 860 utilizing the first fluid inlet pump 840 and into the first fluid circulation path 852. In embodiments, the first volume of fluid is loaded without activating the first fluid circulation pump 848. The first volume of fluid may be provided, at least in part, from fluid container 746A, fluid container 746B, IC fluid container 746, or a combination of these. For example, the first volume of fluid may be circulated through the IC circulation feed loop 753.

As illustrated in FIG. 9B, a volume of the first fluid circulation path 852 may be comprised of a number of volumes of its portions. For example, a first portion of the volume may be an intracapillary space (when the cell growth chamber is a hollow fiber membrane bioreactor) of the cell growth chamber 844. A second portion of the volume may be from connection 860B to an inlet port 844A of the cell growth chamber 844. A third portion may be from the connection 860B to an outlet port 844B of the cell growth chamber 844.

Process 800 proceeds to loading of a second volume of fluid 806. The second volume of fluid may comprise media and may be introduced into a portion of the first fluid flow path 860. In embodiments, the second volume may be a predetermined amount selected in order to position 808 the first volume into a first portion of the cell growth chamber 844. In embodiments, the first volume of fluid and the second volume of fluid may be the same. In other embodiments, the first volume of fluid and the second volume of fluid may be different. In yet other embodiments, a sum of the first volume of fluid and the second volume of fluid may be equal to a percentage of a volume of the first fluid circulation path, e.g., path 852 (FIG. 9B).

The second volume of fluid may be provided, at least in part, from fluid container 768A, fluid container 768B, EC fluid container 768, or a combination of these. For example, the second volume of fluid may be circulated through the IC circulation feed loop 753. For example, the second volume of fluid may be provided from two or more connected fluid containers, as described with respect to FIGS. 7A-7C.

In order to position the first volume of fluid, the second volume of fluid has to be enough to push the first volume into the desired position in the cell growth chamber 844. The second volume of fluid may therefore, in embodiments, be about as large as the volume of the first fluid circulation path 852 between connection 860B and the inlet port 844A. As may be appreciated, this would push the first volume of fluid with the cells into a position within the cell growth chamber 844.

In other embodiments, the first volume of fluid (with cells) may be positioned 808 about a central region 866 of the cell growth chamber 844. In these embodiments, the second volume may be about as large a sum of the volume of the first fluid circulation path 852, between connection 860B and the inlet port 844A and the volume of the first fluid circulation path made up by the cell growth chamber 844 (e.g., the volume of the intracapillary space) that would not be occupied by the first volume of fluid when positioned in the cell growth chamber. For example, in one embodiment, the first volume of fluid (with the cells) may be 50 ml. The cell growth chamber may have a volume of, for example, 124 ml. When the first volume is positioned around the central region 866, it will occupy the 50 ml around the central region 866, leaving 74 ml, which is on either side of the central region 866. Accordingly, 50% of 74 ml (or 37 ml) may be added to the volume between connection 860B and inlet port 844A to position the 50 ml of the first volume around the central region 866.

Positioning 808 the first volume may in embodiments, involve adding additional volumes of fluid to position the first volume with cells in the cell growth chamber. For example, if the desired position of the first volume is not achieved with the second volume, additional fluid may be added to position the first volume at 808.

Process 800 proceeds to the query 810 to determine whether the first volume should be repositioned. For example, in embodiments, the first volume may be positioned closer to inlet port 844A. If it is desired to move the first volume closer to central region 866 of cell growth chamber 844, process 800 may loop back to step 808 where additional fluid may be added to the first fluid circulation path to position the first volume of fluid.

If it is determined at query 810 that the first volume does not need to be repositioned, process 800 proceeds to feeding of the cells 812. In embodiments, the cells may be fed using a media that includes a number of compounds such as glucose, proteins, growth factors, reagents, or other nutrients. In embodiments, feeding of the cells 812 may involve activating inlet pumps and circulation pumps (e.g., pumps 840 and 848) to deliver media with nutrients to the cells in the cell growth chamber 844. Some embodiments provide for the cells to be maintained in the cell growth chamber 844 during step 812, as described below. These embodiments may involve activating pumps, e.g., inlet pumps and circulation pumps (e.g., pumps 840 and 848, as described below) so that flow of fluid into the cell growth chamber 844 may be from both directions such as from the inlet port 844A and the outlet port 844B into the cell growth chamber 844.

For example, the media may be delivered to the cells from one or more connected fluid containers, as described with respect to FIGS. 7A-7C. The inlet pumps and circulation pumps may circulate, and re-circulate, the media through the IC circulation feed loop 753 or the EC circulation feed loop 752 from both directions, such as from the inlet port 844A and the outlet port 844B into the cell growth chamber 844.

Process 800 then proceeds to the expansion of the cells 814 where the cells may be expanded or grown. While step 814 is shown after step 812, step 814 may occur before, or simultaneous with, step 812, according to embodiments. Cells may then be removed 816 from the cell growth chamber and collected in a storage container. In embodiments, step 816 may involve a number of sub-steps. For example, the cells may be circulated by the circulation pump (e.g., pump 848) before they are collected and stored in a container. Process 800 terminates at END operation 830.

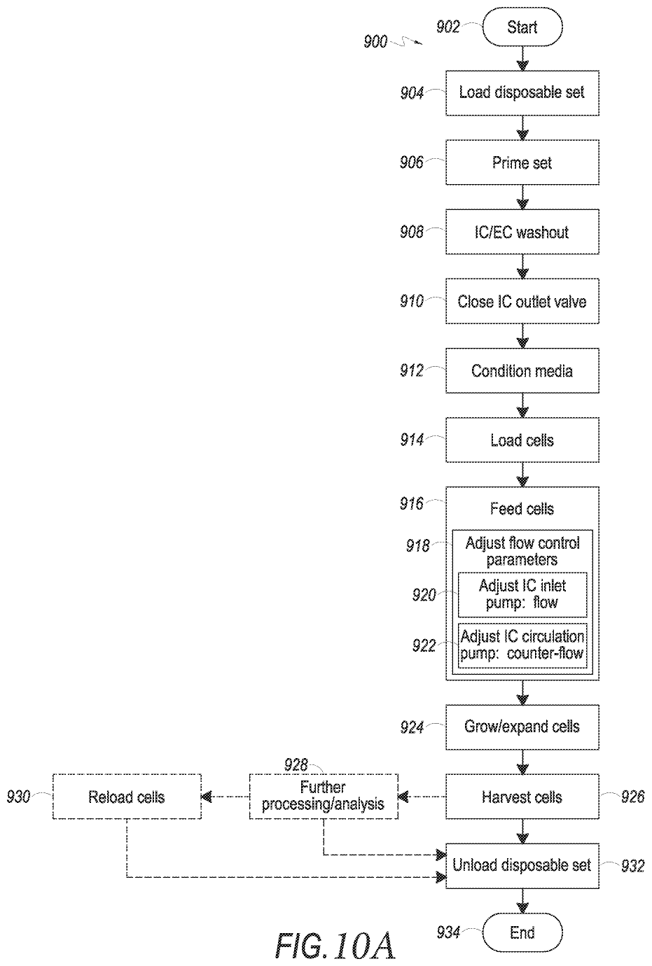

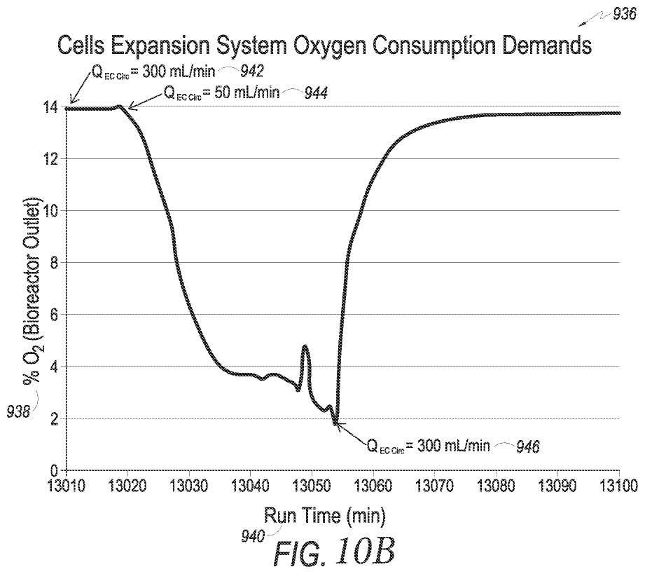

Next, FIG. 10A illustrates example operational steps of a process 900 for retaining cells in a bioreactor of a cell expansion system, such as CES 500 (e.g., FIGS. 5B & 5C) or CES 700 (e.g., FIGS. 7A-7C), in accordance with embodiments of the present disclosure. As discussed above, cells residing in the headers of the bioreactor or in the IC loop outside of the bioreactor may not receive proper gas exchange and nutrient exchange, which may result in cell aggregation and death. In an embodiment, the bioreactor provides gas exchange and nutrient exchange through the semi-permeable hollow fiber membrane. It can be important that such exchange is efficient as the surface area to volume ratio in a cell expansion system comprising a hollow fiber membrane may be significantly larger than that of other cell culturing methods (e.g., about 15 times that of cell culture flasks, for example). Such efficiency may be accomplished by minimizing the diffusion distance for media constituents able to pass the membrane surface where exchange takes place.

For example, FIG. 10B illustrates a graph 936 of the oxygen consumption demands placed on a cell expansion system, such as the Quantum® Cell Expansion System or the Quantum Flex® Cell Expansion System during cell proliferation (approximately 3E+09 T-cells present in the bioreactor). FIG. 10B depicts the percentage (%) of oxygen (O₂) at the bioreactor outlet 938. For example, a sensor to measure oxygen levels may be placed at the EC outlet port of the bioreactor, according to an embodiment. In another embodiment, a sensor to measure oxygenation may be placed at the IC outlet port of the bioreactor. The percentage of O₂ is measured against the Run Time (e.g., in minutes) 940. To maximize the oxygen supply to the cells, the EC circulation flow rate, Q_{EC Circ} may be set to 300 mL/min (942), according to an embodiment. FIG. 10B shows a change in oxygenation when the EC circulation rate is dropped from 300 mL/min (942) to 50 mL/min (944). For example, the cells may consume oxygen (O₂) in the media as the media travels across the bioreactor. Fluid at 50 mL/min (944) moves more slowly across the bioreactor than fluid at 300 mL/min (942), so there may be a longer time period or greater opportunity for the cells to strip the media of oxygen when the EC circulation rate is at 50 mL/min. The oxygenation then recovers when the EC circulation rate is taken back up to 300 mL/min (946). FIG. 10B shows a possible benefit of keeping the cells in the fibers themselves where gas transfer takes place, as opposed to in the portion of the IC circulation path outside of the bioreactor, for example, where the cells may be deprived of oxygen. It may therefore be beneficial to retain cell, e.g., non-adherent cell, populations inside the hollow fibers of the bioreactor during feeding by directing the flow of media to enter both sides of the bioreactor, e.g., the IC inlet port and the IC outlet port. In an embodiment, an equal distribution of flow to the IC inlet port and the IC outlet port may be used. In another embodiment, a larger flow to the IC inlet port as compared to the IC outlet port may be used and vice versa, depending on where it is desired to locate the cells in the bioreactor, for example.

Returning to FIG. 10A, START operation 902 is initiated and process 900 proceeds to load a disposable set or premounted fluid conveyance assembly (e.g., 210 or 400) onto a cell expansion system 904. The disposable set may include the cell growth chamber 100A or the cell growth chamber 100B, as illustrated in FIGS. 1C, 4B, and 4C. The disposable set may then be primed 906, in which the set may be primed 906 with Lonza Ca²⁺/Mg²⁺-free PBS, for example. In preparation for the loading and seeding of cells, the priming fluid may be exchanged using an IC/EC washout 908. In an embodiment, the PBS in the system may be exchanged for TexMACS GMP Base Medium, for example.

For example, during priming, the fluid may be delivered through the disposable set from one or more connected fluid containers, as described with respect to FIGS. 7A-7C. The fluid may be circulated, and re-circulated, using the fluid containers for additional volume in the fluid loop.

Next, process 900 proceeds to close the IC outlet valve 910. In embodiments, the EC outlet valve may be open to allow for ultrafiltration of fluid added to the hollow fibers of a bioreactor comprising a hollow fiber membrane. The media may next be conditioned 912. Next, process 900 proceeds to load cells 914, e.g., suspension or non-adherent cells, such as T cells or Tregs. In an embodiment, such cells may be loaded 914 by a “load cells centrally without circulation” task. In another embodiment, such cells may be loaded 914 by a “load cells with uniform suspension” task. In other embodiments, other loading tasks and/or loading procedures may be used.

In an embodiment, the cells being loaded 914 may be suspended in a solution comprising media to feed the cells during and after such loading, for example. In another embodiment, such solution may comprise both media for feeding the cells and a soluble activator complex to stimulate the cells, e.g., T cells. Such loading at 914 may occur on Day 0, for example.

Following the loading of cells 914, the cells may be further fed 916. In an example embodiment, the cells may be fed 916 through the EC circulation feed loop 752, the IC circulation feed loop 753, or a combination thereof, as described with reference to FIGS. 7A-7C. During such feeding 916, it may be desired to control the cell residence in the bioreactor itself. Through the adjustment of flow control parameters 918, the cells may be retained in the bioreactor itself instead of losing cells from the bioreactor into the portion(s) of the IC loop outside of the bioreactor, for example, during the expansion phase of growth. By retaining the cells in the bioreactor, cells in the bioreactor may be closer to the IC inlet port, in which such cells may receive the freshest growth media, according to embodiments. On the other hand, cells in the IC loop may be receiving expended or conditioned media which may affect their glycolytic metabolism, for example. In addition, cells in the bioreactor may receive mixed gas (e.g., oxygen, carbon dioxide, and nitrogen) input from a gas transfer module (GTM) by diffusion from the EC loop circulation, whereas cells in other portions of the IC loop may not receive such mixed gas, according to an embodiment. It should be noted that while embodiments may provide for retaining the cells in the bioreactor itself, other embodiments may provide for maintaining the cells in any location allowing for improved nutrient delivery and/or gas exchange. Embodiments thus provide for the use of other locations for retaining cells, or controlling the residence of cells, without departing from the spirit and scope of the present disclosure.

Returning to FIG. 10A and process 900, the loss of cells from the hollow fiber membrane bioreactor may be reduced by matching, or closely or substantially matching, the IC circulation pump rate to the IC inlet pump rate, but in the opposite direction, in accordance with embodiments. The IC inlet pump may be adjusted at 920 to produce a first flow rate or volumetric flow rate, and the IC circulation pump may be adjusted at 922 to produce a second counter-flow rate or second counter-volumetric flow rate, in which a volumetric flow rate or fluid flow rate or rate of fluid flow or flow rate may be considered as the volume of fluid which passes per unit time (may be represented by the symbol “Q”). For example, an IC inlet pump rate of 0.1 mL/min may be matched, or closely or substantially matched, to a complementary IC circulation pump rate of −0.1 mL/min to maintain cells in the bioreactor during the growth phase of the cell culture, which may be Days 4-7, for example, in embodiments. Alternatively, an IC inlet pump rate of 0.01 mL/min may be matched, or closely or substantially matched, to a complementary IC circulation pump rate of −0.01 mL/min to maintain cells in the bioreactor during the growth phase of the cell culture, which may be Days 4-7, for example, in embodiments. Such pump adjustment 918 may allow for counteracting any forces associated with a loss of cells from the IC outlet port of the bioreactor, for example.

Next, the cells may be allowed to grow or expand 924. The cells are not limited to growing or expanding at step 924, but, instead, the cells may also expand during step(s) 914, 916, 918, 920, 922, for example. Process 900 may next proceed to harvest operation 926, in which the cells may be transferred to a harvest bag(s) or container(s). The disposable set may then be unloaded at 932 from the cell expansion system, and process 900 then terminates at END operation 934.

Alternatively, from harvest operation 926, process 900 may optionally proceed to allow for further processing/analysis 928. Such further processing 928 may include characterization of the phenotype(s), for example, of the harvested cells. From optional further processing/analysis step 928, process 900 may proceed to optionally reload any remaining cells 930. Process 900 may then proceed to unload the disposable set 932, and process 900 may then terminate at END operation 934. Alternatively, process 900 may proceed from further processing/analysis step 928 to unload disposable set 932. Process 900 may then terminate at END operation 934.

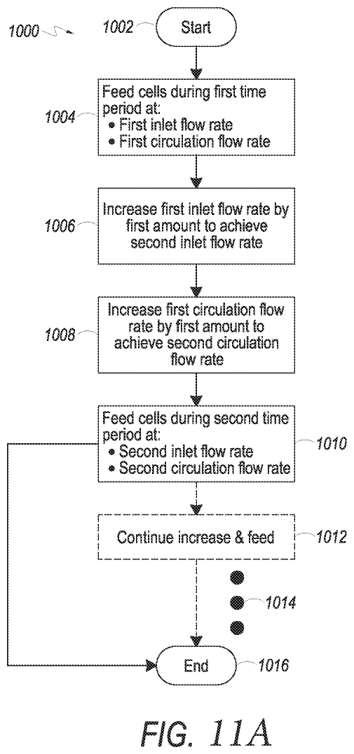

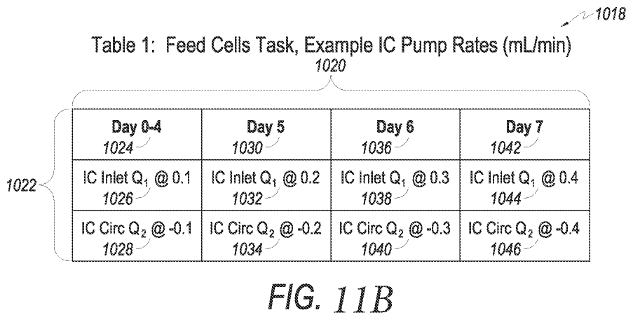

Next, FIG. 11A illustrates example operational steps of a process 1000 for feeding cells that may be used with a cell expansion system, such as CES 700 (e.g., FIGS. 7A-7C), in accordance with embodiments of the present disclosure. START operation 1002 is initiated, and process 1000 proceeds to load a disposable set onto the cell expansion system, prime the set, perform an IC/EC washout, condition media, and load cells, e.g., suspension or non-adherent cells, for example. The disposable set may include the cell growth chamber 100A or the cell growth chamber 100B, as illustrated in FIGS. 1C, 4B, and 4C. Next, process 1000 proceeds to feed the cells during a first time period 1004. In an example embodiment, the cells may be fed 916 through the EC circulation feed loop 752, the IC circulation feed loop 753, or a combination thereof, as described with reference to FIGS. 7A-7C. In embodiments, a first inlet flow rate and a first circulation flow rate may be used. As an example, a first IC inlet flow rate and a first IC circulation flow rate may be used, in which the first IC inlet flow rate may be controlled by the IC inlet pump, e.g., first pump, and the first IC circulation flow rate may be controlled by the IC circulation pump, e.g., second pump. In an example embodiment, the IC inlet pump (754) may cause a volumetric flow rate of 0.1 mL/min to enter the IC inlet port (701A) of the bioreactor (701) with the IC circulation pump (712) causing a complementary IC circulation volumetric flow rate or fluid flow rate of −0.1 mL/min to enter the IC outlet port (701B) of the bioreactor (701), in which the negative symbol (“−”) used in −0.1 mL/min, for example, indicates a direction of the IC circulation pump (712) to cause or produce a counter-flow rate to maintain cells in the bioreactor during the growth phase of the cell culture.

During the feeding of the cells and the use of the IC pumps during feeding to control cell residence in the bioreactor through flow and counter-flow properties, the cells continue to grow and expand. As a result, the cells may demand additional media, e.g., glucose and/or cell growth formulated media, to support the expanding population. In an example embodiment, multiple fluid containers 768A and 768B may be connected to provide media for circulation and recirculation through the system, preventing the need to control lactate values. In alternative embodiments, efforts may be made to control lactate values of the expanding cell population. In embodiments, cell culture lactate values may be maintained at or below about 20 mmol/L, at or below about 15 mmol/L, at or below about 10 mmol/L, or even at or below about 7 mmol/L. In other embodiments, rate(s) of media addition, for example, and/or other settings may be controlled to attempt to maintain the lactate levels≤about 5 mmol/L, for example, to improve cell growth and viability. Other concentrations may be used in other embodiments.

In an example embodiment, because the fluid containers 768A and 768B or the fluid containers 746A and 746B are connected, pump rate does not need to be controlled to control lactate values. In an alternative example embodiment, an effort may be made to control lactate values at ≤about 7 mmol/L by concurrently increasing both the IC inlet (+) pump rate and IC circulation (−) pump rate from ±0.1 to ±0.4 mL/min within the lumen of the hollow fiber membrane over multiple time periods, e.g., days (Days 4-8), according to embodiments. For example, FIG. 11B provides a table 1018 of example IC pump rates for feeding using a “feed cells” task, for example, with a cell expansion system (e.g., CES 500). Table 1018 provides example time periods 1020, e.g., Days, versus example IC pump rates 1022 to produce volumetric flow rates to both sides of the bioreactor to maintain cells in the bioreactor. For example, Days 0-4 (1024) may use an IC inlet or input pump rate of 0.1 mL/min (1026) and an IC circulation pump rate of −0.1 mL/min (1028); Day 5 (1030) may use an IC inlet pump rate of 0.2 mL/min (1032) and an IC circulation pump rate of −0.2 mL/min (1034); Day 6 (1036) may use an IC inlet pump rate of 0.3 mL/min (1038) and an IC circulation pump rate of −0.3 mL/min (1040); and Day 7 (1042) may use an IC inlet pump rate of 0.4 mL/min (1044) and an IC circulation pump rate of −0.4 mL/min (1046). While table 1018 of FIG. 10B provides example pump rates of ±0.1 to ±0.4 mL/min for feeding the cells while retaining the cells in the bioreactor during the growth phase of the cell culture, other pump rates and resulting flow rates may be used according to embodiments without departing from the spirit and scope of the present disclosure. For example, while increments of ±0.1 mL/min for increasing the feed flow rate are shown in this example, other increments, e.g., ±0.005 mL/min, ±0.05 mL/min, etc., may be used to increase the feed flow rate in embodiments. The time periods, e.g., Days, and pump rates in table 1018 of FIG. 11B are offered merely for illustrative purposes and are not intended to be limiting. For example, the time periods and pump rates in table 1018 of FIG. 11B may be provided for the cell growth chamber 100B. The pump rates in table 1018 of FIG. 11B may be reduced to 10% of the described pump rates for the cell growth chamber 100A.

Returning to FIG. 11A, process 1000 proceeds from feeding the cells during a first time period 1004 to increasing the first inlet flow rate by a first amount to achieve a second inlet flow rate 1006. For example, as in the embodiment depicted in FIG. 11B as discussed above, the IC inlet pump rate (+) may increase from 0.1 mL/min to 0.2 mL/min to produce an IC inlet flow rate of 0.2 mL/min. Further, the IC circulation pump rate (−) may concurrently increase from −0.1 mL/min to −0.2 mL/min to produce an IC circulation flow rate of −0.2 mL/min. (The pump rates may be reduced to 10% of the described pump rates for the cell growth chamber 100A) The first circulation flow rate is thus increased by the first amount to achieve the second circulation flow rate 1008. The cells may then be fed during a second time period at the second inlet flow rate and at the second circulation flow rate at 1010 to maintain the cells in the bioreactor and outside of the headers and outside of the portion of the IC circulation path outside of the bioreactor, for example. Following the second time period of feeding 1010, process 1000 may terminate at END operation 1016 if it is not desired to continue feeding and/or expanding the cells, for example. Alternatively, process 1000 may optionally continue to increase, or otherwise change, the feeding flow rates 1012. There may be any number of feeding time periods, as represented by ellipsis 1014. Following the desired number of feeding time periods 1014, process 1000 then terminates at END operation 1016. While FIGS. 10A and 10B and process 1000 show “increases” in the flow rates, other adjustments to the flow rates may be made. For example, the flow rates may decrease or remain substantially the same from one feeding period to the next. Considerations, such as metabolic activity, for example, may determine how flow rates are adjusted. The “increases” in flow rates in FIGS. 10A and 10B are offered merely for illustrative purposes and are not intended to be limiting.

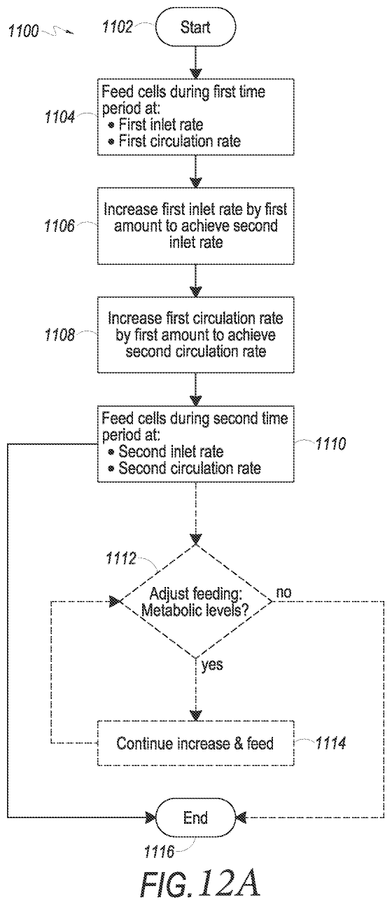

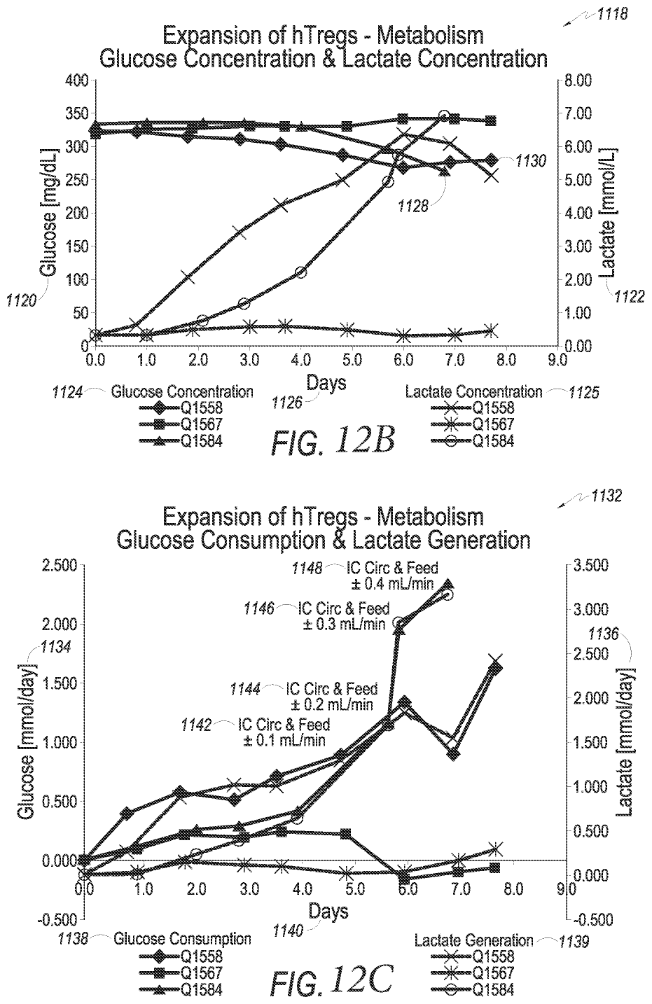

Turning to FIG. 12A, example operational steps of a process 1100 for feeding cells that may be used with a cell expansion system, such as CES 500 (e.g., FIGS. 5B & 5C), are provided in accordance with embodiments of the present disclosure. START operation is initiated 1102, and process 1100 proceeds to load a disposable set onto the cell expansion system, prime the set, perform an IC/EC washout, condition media, and load cells, e.g., suspension or non-adherent cells, for example. Next, process 1100 proceeds to feed the cells during a first time period 1104. In embodiments, a first inlet rate or flow rate and a first circulation rate or flow rate may be used. As an example, a first IC inlet flow rate and a first IC circulation flow rate may be used, in which the first IC inlet flow rate may be controlled by the IC inlet pump, e.g., first pump, and the first IC circulation flow rate may be controlled by the IC circulation pump, e.g., second pump. In an example embodiment, the IC inlet pump (554) may cause a volumetric flow rate or fluid flow rate of 0.1 mL/min to enter the IC inlet port (501A) of the bioreactor (501) with the IC circulation pump (512) causing a complementary IC circulation flow rate of −0.1 mL/min to enter the IC outlet port (501B) of the bioreactor (501), in which the negative symbol (“−”) used in −0.1 mL/min, for example, indicates a direction of the IC circulation pump (512) to cause or produce a counter-flow rate to maintain cells in the bioreactor during the growth phase of the cell culture. For example, the flow rates described may be suitable for cell growth chamber 100B. The described flow rates may be reduced to 10% for cell growth chamber 100A.

During the feeding of the cells and the use of the IC pumps to control cell residence in the bioreactor through flow and counter-flow properties, the cells continue to grow and expand. As a result, the cells may demand additional media, e.g., glucose and/or cell growth formulated media, to support the expanding population. In an example embodiment, multiple fluid containers 768A and 768B may be connected to provide media for circulation and recirculation through the system, preventing the need to control lactate values. In alternative embodiments, efforts may be made to control lactate values of the expanding cell population. In an example embodiment, an effort may be made to control lactate values at ≤about 7 mmol/L, for example, by concurrently increasing both the IC inlet (+) pump rate and IC circulation (−) pump rate from ±0.1 to ±0.4 mL/min within the lumen of the hollow fiber membrane over multiple days, e.g., Days 4-8, according to embodiments. For example, see FIG. 11B, table 1018, and the discussion above, for example pump rates for feeding. As noted, while table 1018 of FIG. 11B provides pump rates of ±0.1 to ±0.4 mL/min for feeding the cells while retaining the cells in the bioreactor during the growth phase of cell culturing, other pump rates and resulting flow rates may be used according to embodiments without departing from the spirit and scope of the present disclosure. The time periods, e.g., Days, and pump rates in table 1018 of FIG. 11B are offered merely for illustrative purposes and are not intended to be limiting. For example, the time periods and pump rates in table 1018 of FIG. 11B may be provided for the cell growth chamber 100B. The pump rates in table 1018 of FIG. 11B may be reduced to 10% of the described pump rates for the cell growth chamber 100A.

Returning to FIG. 12A, process 1100 proceeds from feeding the cells during a first time period 1104 to increasing the first inlet rate or flow rate by a first amount to achieve a second inlet rate or flow rate 1106. For example, as in the embodiment depicted in FIG. 11B as discussed above, the IC inlet pump rate (+) may increase from 0.1 mL/min to 0.2 mL/min to produce an IC inlet flow rate of 0.2 mL/min. Further, the IC circulation pump rate (−) may concurrently increase from −0.1 mL/min to −0.2 mL/min to produce an IC circulation flow rate of −0.2 mL/min. (The pump rates may be reduced to 10% of the described pump rates for the cell growth chamber 100A) The first circulation rate or flow rate is thus increased by the first amount to achieve the second circulation rate or flow rate 1108. The cells may then be fed during a second time period at the second inlet rate or flow rate and at the second circulation rate or flow rate 1110 to maintain the cells in the bioreactor and outside of the headers or the portion of the IC circulation path outside of the bioreactor, for example. Following the second period of feeding 1110, process 1100 may terminate at END operation 1116 if it is not desired to continue feeding and/or expanding the cells, for example.

Alternatively, process 1100 may optionally determine whether to adjust the feeding rates or flow rates based on metabolic activity, in which process 1100 proceeds to optional query 1112 to determine whether to adjust feeding based on metabolic levels. For example, monitoring the glucose and/or lactate levels can facilitate the adjustment of cell expansion system media flow rates, e.g., IC media flow rate, to support cell, e.g., Treg, expansion in a bioreactor, such as a hollow fiber bioreactor, for example.

As shown in FIGS. 12B and 12C, embodiments provide for controlling the lactate values of cell expansion runs or procedures involving the expansion of hTregs, for example. Graphs 1118 and 1132 indicate that measurements of glucose and lactate levels may be used to adjust cell expansion system media flow rates, e.g., IC media flow rates, to support the expansion of Tregs, for example. For example, FIG. 12B provides a graph 1118 showing the metabolisms of expanding hTregs, in which such cell expansion may occur in a cell expansion system, such as the Quantum® Cell Expansion System. The glucose concentration (mg/dL) 1120 and lactate concentration (mmol/L) 1122 are shown for various cell expansion runs 1124 and 1125 across periods of time, e.g., Days, 1126. Throughout these Treg runs 1124 and 1125, an effort may be made to control the lactate values of the expanding cell population to values≤about 7 mmol/L by concurrently increasing both the IC inlet (+) pump rate and IC circulation pump rate from ±0.1 to ±0.4 mL/min, for example, within the lumen of the hollow fiber membrane over Days 4-8, for example. In other embodiments, other pump rates may be used. As shown, the lowest glucose levels during the Treg cell expansions may range from a concentration 1128 of 264 mg/dL on Day 7 (Q1584) to a concentration 1130 of 279 mg/dL on Day 8 (Q1558), according to the embodiments shown. As depicted, the base glucose concentration, in the cell growth formulated medium for runs 1124 may range from 325 mg/dL to 335 mg/dL, for example. In other embodiments, it may be desired to maintain the lactate levels≤about 5 mmol/L to improve cell growth and viability. In embodiments, graphical user interface (GUI) elements may be used to control the rate of media addition and to maintain the lactate metabolic waste product from glycolysis below a defined level during the expansion of cells.

Turning to FIG. 12C, graph 1132 shows the metabolisms of expanding hTregs, in which such cell expansion may occur in a cell expansion system, such as the Quantum® Cell Expansion System. The glucose consumption (mmol/day) 1134 and lactate generation (mmol/day) 1136 are shown for various cell expansion runs 1138 and 1139 across periods of time 1140, e.g., Days. To control lactate values at ≤about 7 mmol/L, for example, concurrent increases may be made to the IC inlet (+) pump rate and IC circulation (−) pump rates from ±0.1 to ±0.4 mL/min, in an embodiment. For example, graph 1132 shows IC circulation and IC feed rates of ±0.1 mL/min (1142); ±0.2 mL/min (1144); ±0.3 mL/min (1146); and ±0.4 mL/min (1148). Other embodiments may use other flow rates. The flow rates used and shown in FIGS. 12B and 12C are offered for purposes of illustration and are not intended to be limiting. It is noted that while positive (+) may be shown for the direction of the IC inlet pump and negative (−) may be shown for the direction of the IC circulation pump, such directions are offered for illustrative purposes only, in which such directions depend on the configurations of the pumps used.

Returning to FIG. 12A and optional query 1112, if it is not desired to measure metabolic activity and/or adjust feeding levels based on such measurement(s), process 1100 proceeds “no” to END operation 1116, and process 1100 terminates. For example, where multiple feed containers are used, as described with respect to FIGS. 7A-7C, media may be circulated and re-circulated, preventing a need to measure metabolic activity and/or adjust feeding levels. Alternatively, where it is desired to adjust feeding levels based on metabolic activity, process 1100 proceeds “yes” to optional step 1114 to continue to increase the rate of media addition and feed the expanding cell population. While step 1114 is shown as one step, this step may involve numerous adjustments to the media addition rate, such as increasing the IC inlet flow rate and increasing the IC circulation flow rate, for example. Step 1114 is shown as one step only for illustrative purposes and is not intended to be limiting. Following any adjustments to the rate of media addition, process 1100 proceeds to optional query 1112 to determine whether to continue measuring metabolic levels and/or adjust feeding. If it is not desired to continue measuring metabolic activity and/or adjusting feeding levels based on metabolic activity, process 1100 proceeds “no” to END operation 1116, and process 1100 terminates. While FIG. 11A and process 1100 show “increases” in the rates or flow rates, other adjustments to the flow rates may be made. For example, the rates or flow rates may decrease or remain substantially the same from one feeding period to the next. The type of adjustments which may be made may depend on the metabolic activity assessment of the growing cell population. The “increases” in flow rate in FIG. 11A, for example, are offered merely for illustrative purposes and are not intended to be limiting.

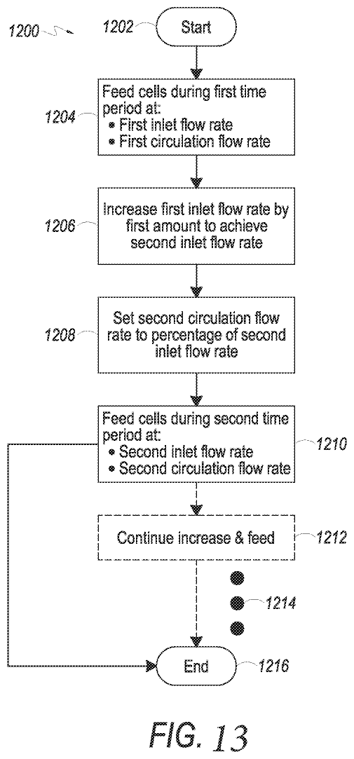

Next, FIG. 13 illustrates example operational steps of a process 1200 for retaining cells in a location during feeding using a cell expansion system, such as CES 500 (e.g., FIGS. 5B &5C) or CES 700 (e.g. FIGS. 7A-7C), in accordance with embodiments of the present disclosure. START operation 1202 is initiated, and process 1200 proceeds to load a disposable set onto the cell expansion system, prime the set, perform IC/EC washout, condition media, and load cells, e.g., suspension or non-adherent cells, for example. The disposable set may include the cell growth chamber 100A or the cell growth chamber 100B, as illustrated in FIGS. 1C, 4B, and 4C. Next, process 1200 proceeds to feed the cells during a first time period 1204. In embodiments, a first inlet flow rate and a first circulation flow rate may be used. As an example, a first IC inlet flow rate and a first IC circulation flow rate may be used, in which the first IC inlet flow rate may be produced and controlled by the IC inlet pump, e.g., first pump, and the first IC circulation flow rate may be produced and controlled by the IC circulation pump, e.g., second pump. In an example embodiment, the IC inlet pump (554) may cause a volumetric flow rate of 0.1 mL/min to enter the IC inlet port (501A) of the bioreactor with the IC circulation pump (512) causing a complementary IC circulation volumetric flow rate or fluid flow rate of −0.1 mL/min to enter the IC outlet port (501B) of the bioreactor (501), in which the negative symbol (“−”) used in −0.1 mL/min, for example, indicates a direction of the IC circulation pump to cause or produce a counter-flow rate to maintain cells in the bioreactor during the growth phase of the cell culture. In another embodiment, the first IC circulation flow rate may be a percentage of the first IC inlet flow rate. For example, the first IC circulation flow rate may be about fifty percent (50%) or about one-half (½), or another percentage or portion according to embodiments, of the first IC inlet flow rate. Alternatively, in an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture.

During the feeding (and expansion) of the cells and the use of the IC pumps, for example, to control cell residence in the bioreactor through flow and counter-flow properties, the cells continue to grow and expand. As a result, the cells may demand additional media, e.g., glucose and/or cell growth formulated media, to support the expanding population. In embodiments, efforts may be made to increase the rate of media addition to feed the expanding cell population. In an example embodiment, an increase in the IC inlet pump rate (+) may cause the IC inlet flow rate to increase by a first amount to achieve a second IC inlet flow rate 1206. For example, the IC inlet flow rate may increase by a first amount of 0.1 mL/min to achieve a second IC inlet flow rate of 0.2 mL/min, according to an embodiment. Other adjustments may be made to the IC inlet flow rate according to other embodiments.

In an example embodiment, the cells may be fed 916 through the EC circulation feed loop 752, the IC circulation feed loop 753, or a combination thereof, as described with reference to FIGS. 7A-7C.

Next, the second IC circulation flow rate may be set, or adjusted or configured, to equal a percentage, or portion, or fraction of the second IC inlet flow rate 1208, according to embodiments. For example, the second IC circulation flow rate may be set, or adjusted or configured, to equal about fifty percent (50%) or about one-half (½), or another percentage or portion according to embodiments, of the value of the IC inlet flow rate, according to an embodiment. Depending on the value of the first IC circulation flow rate, an adjustment to the IC circulation pump rate (−) may cause the second IC circulation flow rate to increase, according to an embodiment. In another embodiment, an adjustment may be made to the IC circulation pump rate to produce or cause the second IC circulation flow rate to decrease such that the IC circulation flow rate may be substantially equal to a pre-defined percentage or pre-defined fraction of the second IC inlet flow rate. In yet another embodiment, no adjustment may be made to the IC circulation pump rate. For example, where the first IC inlet flow rate equals 0.1 mL/min, and the first IC circulation flow rate equals −0.1 mL/min, if the second IC inlet flow rate is increased to 0.2 mL/min, the second IC circulation flow rate may be set to (−½)*(second Q_{IC Inlet}) (where Q_{IC Inlet} is the IC inlet flow rate) or (−½)*(0.2 mL/min), which provides for a second Q_{IC Circ} (where Q_{IC Circ} is the IC circulation flow rate) of −0.1 mL/min, and no adjustment to the IC circulation pump rate may be made to achieve such second Q_{IC Circ}.

Turning to FIGS. 5B and 5C, such figures depict operational configurations of cell expansion system 500 showing fluid movement in the first circulation path, in accordance with embodiments of the present disclosure. The configurations of FIGS. 5B and 5C show a split, for example, in the flow rate to the IC inlet port, e.g., first port, and to the IC outlet port, e.g., second port, to keep the cells in the cell growth chamber or bioreactor. As discussed with respect to CES 500 above, in the IC loop or first circulation path 502, fluid may be initially advanced by the IC inlet pump 554. Such fluid may be advanced in a first direction, such as a positive direction, for example. Fluid may flow into cell growth chamber or bioreactor 501 through IC inlet port 501A, through hollow fibers in cell growth chamber or bioreactor 501, and may exit via IC outlet port 501B. Media may flow through IC circulation pump 512 which may be used to control the rate of media flow. IC circulation pump may pump the fluid in a first direction or second direction opposite the first direction. IC outlet port 501B may be used as an inlet in the reverse direction, for example. In an embodiment, the IC circulation pump 512 may pump the fluid in a direction opposite the direction of the IC inlet pump, for example. As an example, the direction of the IC inlet pump may be positive (+), and the direction of the IC circulation pump may be negative (−) to cause a counter-flow such that fluid may enter both sides of the bioreactor to keep the cells in the bioreactor.

In an embodiment, a first portion of fluid may branch at connection 517 to flow into the IC inlet port (501A) of the bioreactor 501. In an embodiment, the IC circulation pump 512 may operate at a pump rate that may be matched, or closely or substantially be matched, to the IC inlet pump 554 rate, but in the opposite direction, such that a second portion of fluid may branch at connection 517 to flow into the IC outlet port (501B) of the bioreactor 501. For example, an IC inlet pump rate of +0.1 mL/min may be matched, or closely or substantially be matched, to a complementary IC circulation pump rate of −0.1 mL/min to maintain cells in the bioreactor during the growth phase of the cell culture. Alternatively, an IC inlet pump rate of less than +0.1 mL/min, or about +0.01 mL/min, may be matched, or closely or substantially be matched, to a complementary IC circulation pump rate of less than −0.1 mL/min, or about −0.01 mL/min, to maintain cells in the bioreactor during the growth phase of the cell culture. Furthermore, in additional embodiments, intracapillary harvest synchronization, as described herein, may be followed during the growth phase of the cell culture. Such pump adjustment tactic during the feeding may counteract forces associated with the loss of cells from the IC outlet port. Such type of feeding using pump adjustments to cause flow and counter-flow into the IC inlet port (501A) and IC outlet port (501B), respectively, of the bioreactor may be referred to as a modified feeding method, according to an embodiment. In another example embodiment, the IC circulation pump rate may be adjusted to cause a flow rate into the IC outlet port (501B) that may be equal to about fifty percent (50%) or about one-half (½), or another percentage or fraction in other embodiments, of the IC inlet flow rate, but in the opposite direction. For example, with an IC inlet pump rate of 0.4 mL/min, an IC circulation pump rate may be set, or configured or adjusted, to about −0.2 mL/min. Other percentages or portions may be used in other embodiments.

FIGS. 5B and 5C illustrate operational configurations showing fluid movement in CES 500, in which the flow and counter-flow rates to keep the cells in the cell growth chamber or bioreactor 501 are shown, according to embodiments. For example, such flow rates are illustrated in FIG. 5B as “X” flow rate 503; “(−½) X” flow rate 505 (where the negative symbol (“−”) is an indication of direction, in which a direction of flow rate 505 is shown by the directional arrow in FIG. 5B); and (½) X” flow rate 507, in which about ½, or a first portion, of the flow rate branches at connection 517 to enter IC inlet port (501A) of bioreactor 501, and about ½, or a second portion, of the flow rate branches at connection 517 to enter the IC outlet port (501B) of bioreactor 501, according to embodiments. As shown, the sum of the first portion and the second portion may be substantially equal to the total flow rate 503, in which the total flow rate 503 is pumped by IC inlet pump 554. Depending on the flow and counter-flow rates which may be used to keep the cells in the bioreactor or cell growth chamber 501, other types of fractions or percentages of the IC inlet pump rate may be used to set, or configure or adjust, the IC circulation pump. As such, FIG. 5C illustrates such flow rates as “X” flow rate 511; “−(y %)*X” flow rate 513 (where the negative symbol (“−”) is an indication of direction, in which a direction of flow rate 513 is shown by the directional arrow in FIG. 5C); and “(100%−y %)*X” flow rate 515, where “y” equals a numeric percentage, according to embodiments. In embodiments, the sum of flow rate 513 and flow rate 515 is substantially equal to flow rate 511.

In an embodiment, all, or substantially all, of the flow from the first fluid flow path 506 may flow to IC inlet port (501A) of bioreactor 501 from connection 517, for example. In another embodiment, all, or substantially all, of the flow from the first fluid flow path 506 may flow to IC outlet port (501B) of bioreactor 501 from connection 517. In yet another embodiment, a first portion of the flow from first fluid flow path 506 may flow from connection 517 to IC inlet port (501A), and a second portion of the flow from the first fluid flow path 506 may flow from connection 517 to IC outlet port (501B). In embodiments, the percentage of the IC inlet flow rate at which the IC circulation flow rate may be set may range from about 0 percent to about 100 percent. In other embodiments, the percentage may be between about 25 percent and about 75 percent. In other embodiments, the percentage may be between about 40 percent and about 60 percent. In other embodiments, the percentage may be between about 45 percent and about 55 percent. In embodiments, the percentage may be about 50 percent. It is to be understood that the operational configurations shown in FIGS. 5B and 5C represent possible configurations for various operations of the cell expansion system, and modifications to the configurations shown are within the scope of the one or more present embodiments.

Returning to FIG. 13, the cells may be fed (and continue expanding) during a second time period at the second inlet flow rate and at the second circulation flow rate 1210 to maintain the cells in the bioreactor and outside of the headers or portion of the IC circulation path outside of the bioreactor, for example. Following the second period of feeding 1210, process 1200 may terminate at END operation 1216 if it is not desired to continue feeding and/or expanding the cells, for example. Alternatively, process 1200 may optionally continue to increase, or otherwise change, the feeding flow rates 1212. There may be any number of feeding time periods, as represented by ellipsis 1214. Following the desired number of feeding time periods 1214, process 1200 may then terminate at END operation 1216. While FIG. 13 and process 1200 show “increases” in the flow rates, other adjustments to the flow rates may be made. For example, the flow rates may decrease or remain substantially the same from one feeding period to the next. Considerations, such as metabolic activity, for example, may determine how flow rates are adjusted. The “increases” in flow rates in FIG. 13 are offered merely for illustrative purposes and are not intended to be limiting.

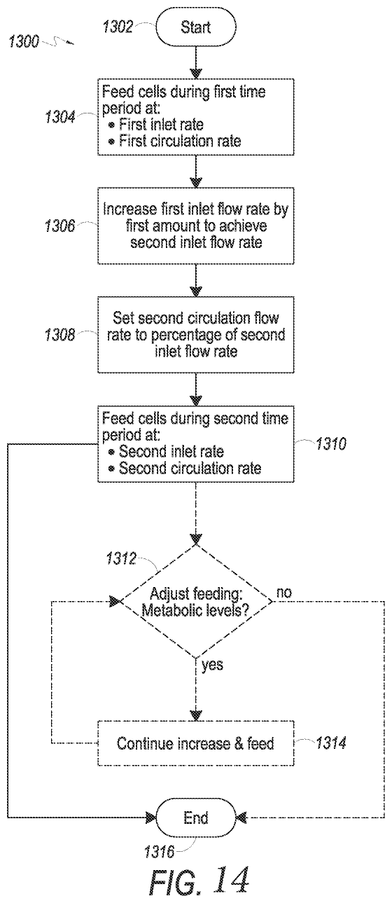

Turning to FIG. 14, example operational steps of a process 1300 for feeding cells while retaining the cells in a first location, e.g., in the bioreactor, that may be used with a cell expansion system, such as CES 500 (e.g., FIGS. 5B & 5C) or CES 700 (e.g., FIGS. 7A-7C), are provided in accordance with embodiments of the present disclosure. START operation 1302 is initiated, and process 1300 proceeds to load a disposable set onto the cell expansion system, prime the set, perform IC/EC washout, condition media, and load cells, e.g., suspension or non-adherent cells, for example. Next, process 1300 proceeds to feed the cells during a first time period 1304. In embodiments, a first inlet flow rate and a first circulation flow rate may be used. As an example, a first IC inlet flow rate and a first IC circulation flow rate may be used, in which the first IC inlet flow rate may be produced and controlled by the IC inlet pump, e.g., first pump, and the first IC circulation flow rate may be produced and controlled by the IC circulation pump, e.g., second pump. In an example embodiment, the first IC inlet pump rate may cause a volumetric flow rate of 0.1 mL/min to enter the IC inlet port of the bioreactor with a complementary IC circulation pump rate of −0.1 mL/min causing a volumetric flow rate of −0.1 mL/min to enter the IC outlet port of the bioreactor, in which the negative symbol (“−”) used in −0.1 mL/min, for example, indicates a direction of the IC circulation pump (512) to cause or produce a counter-flow rate to maintain cells in the bioreactor during the growth phase of the cell culture. Alternatively, in an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture. In another embodiment, the first IC circulation flow rate may be a percentage or fraction or portion of the first IC inlet flow rate. For example, the first IC circulation flow rate may be about fifty percent (50%) or about one-half (½), or another percentage or portion according to embodiments, of the IC inlet flow rate.

During the feeding (and expansion) of the cells and the use of the IC pumps to control cell residence in the bioreactor through flow and counter-flow properties, the cells continue to grow and expand. As a result, the cells may demand additional media, e.g., glucose and/or cell growth formulated media, to support the expanding population. In embodiments, efforts may be made to increase the rate of media addition to feed the expanding cell population. In an example embodiment, an increase in the IC inlet pump rate (+) may cause the IC inlet flow rate to increase by a first amount to achieve a second IC inlet flow rate 1306. For example, the IC inlet flow rate may be increased by a first amount of 0.1 mL/min to achieve a second IC inlet flow rate of 0.2 mL/min, according to an embodiment. Other adjustments may be made to the IC inlet flow rate according to other embodiments.

Next, the second IC circulation flow rate may be set, or configured or adjusted, to equal a percentage or portion or fraction of the second IC inlet flow rate 1308, according to embodiments. For example, the second IC circulation flow rate may be set, or configured or adjusted, to equal about fifty percent (50%) or about one-half (½), or another percentage or portion according to embodiments, of the value of the IC inlet flow rate, according to an embodiment. In an embodiment, all, or substantially all, of the flow from the first fluid flow path 506 may flow to IC inlet port (501A) of bioreactor 501 from connection 517, for example. In another embodiment, all, or substantially all, of the flow from the first fluid flow path 506 may flow to IC outlet port (501B) of bioreactor 501 from connection 517. In yet another embodiment, a first portion of the flow from first fluid flow path 506 may flow from connection 517 to IC inlet port (501A), and a second portion of the flow from the first fluid flow path 506 may flow from connection 517 to IC outlet port (501B). In embodiments, the percentage of the IC inlet flow rate at which the IC circulation flow rate may be set may range from about 0 percent to about 100 percent. In other embodiments, the percentage may be between about 25 percent and about 75 percent. In other embodiments, the percentage may be between about 40 percent and about 60 percent. In other embodiments, the percentage may be between about 45 percent and about 55 percent. In embodiments, the percentage may be about 50 percent.

Depending on the value of the first IC circulation flow rate, an adjustment to the IC circulation pump rate (−) may cause the second IC circulation flow rate to increase, according to an embodiment. In another embodiment, an adjustment may be made to the IC circulation pump rate to cause the second IC circulation flow rate to decrease such that the second IC circulation flow rate may be substantially equals to a pre-defined percentage or pre-defined fraction of the second IC inlet flow rate. In yet another embodiment, no adjustment may be made to the IC circulation pump rate. For example, where the first IC inlet flow rate equals 0.1 mL/min, and the first IC circulation flow rate equals −0.1 mL/min, if the second IC inlet flow rate is increased to 0.2 mL/min, the second IC circulation flow rate may be set to (−½)*(second Q_{IC Inlet}) or (−½)*(0.2 mL/min), which provides for a second Q_{IC Circ} of −0.1 mL/min, and no adjustment to the IC circulation pump rate may be made to achieve such second Q_{IC Circ}, according to an embodiment.

The cells may then be fed (and continue expanding) during a second time period at the second IC inlet flow rate and at the second IC circulation flow rate 1310 to maintain the cells in the bioreactor and outside of the headers or portion of the IC circulation path outside of the bioreactor, for example. Following the second time period of feeding 1310, process 1300 may terminate at END operation 1316 if it is not desired to continue feeding and/or expanding the cells, for example.

In embodiments, a first time period, a second time period, a third time period, a fourth time period, a fifth time period, etc. may each comprise one or more days (and/or hours and/or minutes). For example, a time period may be one (1) to fourteen (14) days, according to embodiments. However, a time period may be less than one (1) day or greater than fourteen (14) days, in other embodiments. In an embodiment, a first time period for feeding may comprise Day 0, Day 1, Day 2, Day 3, Day 4; a second time period for feeding may comprise Day 5; a third time period for feeding may comprise Day 6; and a fourth time period for feeding may comprise Day 7, for example. In another embodiment, a first time period may comprise Day 0, Day 1, and Day 2 (e.g., duration of about 3 days); a second time period may comprise Day 3, Day 4, and Day 5 (e.g., duration of about 3 days); a third time period may comprise Day 6, Day 7, and Day 8 (e.g., duration of about 3 days); a fourth time period may comprise Day 9 and Day 10 (e.g., duration of about 2 days); and a fifth time period may comprise Day 11, Day 12, and Day 13 (e.g., duration of about 3 days). Time periods may be different durations according to embodiments. Each time period may be measured in days, hours, minutes, and/or portions thereof.

Returning to FIG. 14, process 1300 may optionally continue to optional query 1312 to determine whether to adjust feeding rates or flow rates based on metabolic activity or metabolic levels. For the expansion of Tregs, for example, it may be desired to control the lactate values of the expanding cell population to values≤about 7 mmol/L. Where it is desired to maintain the cell culture lactate values at or below about 7 mmol/L, for example, the rate of media addition may be controlled during the expansion of the cells, e.g., regulatory T cells. In other embodiments, it may be desired to maintain the lactate levels≤about 5 mmol/L to improve cell growth and viability. In embodiments, graphical user interface (GUI) elements may be used to control the rate of media addition and to maintain the lactate metabolic waste product from glycolysis below a pre-defined level during the expansion of cells.

At optional query 1312, if it is not desired to measure metabolic activity and/or adjust feeding levels based on such measurement(s), process 1300 proceeds “no” to END operation 1316, and process 1300 terminates. Alternatively, where it is desired to adjust feeding levels based on metabolic activity, process 1300 proceeds “yes” to optional step 1314 to continue to increase the rate of media addition and feed the expanding cell population. While step 1314 is shown as one step, this step may involve numerous adjustments to the media addition rate, such as increasing the IC inlet flow rate and increasing the IC circulation flow rate, for example. Step 1314 is shown as one step only for illustrative purposes and is not intended to be limiting. Following any adjustments to the rate of media addition, process 1300 returns to optional query 1312 to determine whether to continue to measure metabolic levels and/or adjust feeding. If it is not desired to adjust feeding levels based on metabolic activity, process 1300 proceeds “no” to END operation 1316, and process 1300 terminates. While FIG. 13 and process 1300 show “increases” in the rates or flow rates, other adjustments to the flow rates may be made. For example, the rates or flow rates may decrease or remain substantially the same from one feeding period to the next. The type of adjustments which may be made may depend on the metabolic activity assessment of the growing cell population. The “increases” in flow rate in FIG. 13 are offered merely for illustrative purposes and are not intended to be limiting.

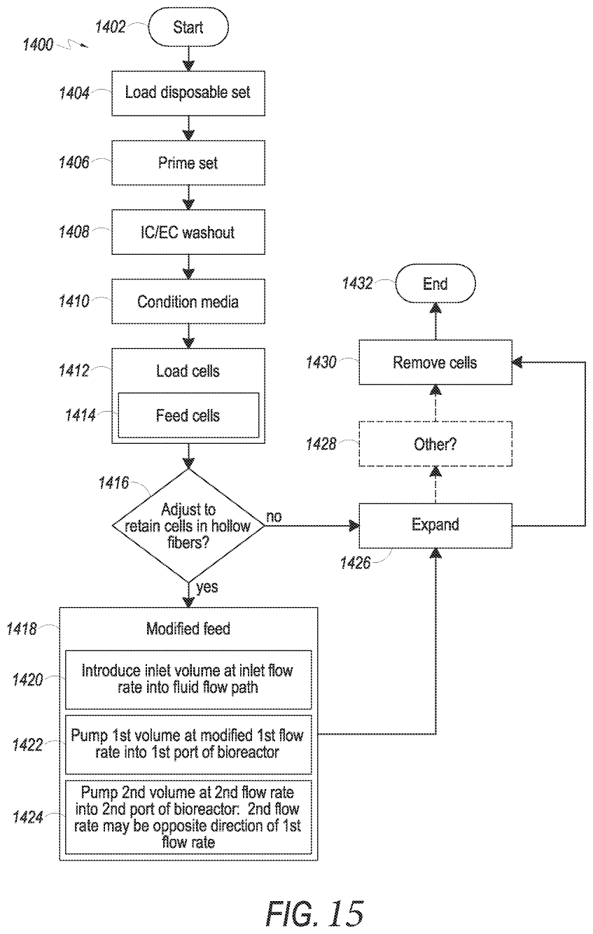

Next, FIG. 15 illustrates example operational steps of a process 1400 for retaining cells during feeding that may be used with a cell expansion system, such as CES 500 (FIGS. 5B & 5C) or CES 700 (FIGS. 7A-7C), in accordance with embodiments of the present disclosure. START operation 1402 is initiated, and process 1400 proceeds to load a disposable tubing set or premounted fluid conveyance assembly (e.g., 210 or 400) 1404 onto the cell expansion system. The disposable set may include the cell growth chamber 100A or the cell growth chamber 100B, as illustrated in FIGS. 1C, 4B, and 4C. Next, the system may be primed 1406. In an embodiment, a user or operator, for example, may provide an instruction to the system to prime by selecting a task for priming, for example. In an embodiment, such task for priming may be a pre-programmed task, for example. Next, the IC/EC washout task may be performed 1408, in which fluid on the IC circulation loop and on the EC circulation loop may be replaced, for example. The replacement volume may be determined by the number of IC volumes and EC volumes exchanged, according to an embodiment.

Next, to maintain the proper or desired gas concentration across the fibers in the bioreactor membrane, the condition media task 1410 may be executed to allow the media to reach equilibrium with the provided gas supply before cells are loaded into the bioreactor. For example, contact between the media and the gas supply provided by the gas transfer module (GTM) or oxygenator may be provided by adjusting the EC circulation rate. The system may then be maintained in a proper or desired state until a user or operator, for example, is ready to load cells into the bioreactor. In an embodiment, the system may be conditioned with media, such as complete media, for example. Complete media may be any media source used for cell growth. In an embodiment, the system may be conditioned with serum-free media, for example. In an embodiment, the system may be conditioned with base media. Any type of media understood by those of skill in the art may be used.

Process 1400 next proceeds to loading cells 1412 into the bioreactor from a cell inlet bag, for example. In an embodiment, the cells in the cell inlet bag may be in solution with media to feed 1414 the cells, for example. In another embodiment, the cells in the cell inlet bag may be in solution both with media to feed 1414 the cells and with a soluble activator complex to stimulate the cells, e.g., T cells or Tregs. In an embodiment, the cells (and feed solution, in embodiments) may be loaded into the bioreactor from the cell inlet bag until the bag is empty. Cells (and feed solution, in embodiments) may be chased from the air removal chamber to the bioreactor. In an embodiment, a “load cells with uniform suspension” task may be executed to load the cells (and feed solution, in embodiments). In another embodiment, a “load cells centrally without circulation” task may be executed to load the cells (and feed solution, in embodiments) into a specific, e.g., central, region of the bioreactor. Other loading methods and/or loading tasks may be used in accordance with embodiments.

Next, process 1400 proceeds to query 1416 to determine whether to use a modified feeding method to retain the cells, e.g., non-adherent or suspension cells, such as T cells or Tregs, in the bioreactor, e.g., hollow fiber bioreactor. For example, it may be desired to locate the cells in the bioreactor itself and out of the headers of the bioreactor or the rest of the IC loop. If it is not desired to use a modified feeding method to retain the cells in the bioreactor itself, process 1400 proceeds “no” to expand the cells 1426, in which the cells may continue to grow/expand using the media that they were initially fed with in step 1414, for example.

On the other hand, if it is desired to retain the cells in the bioreactor itself, process 1400 proceeds “yes” to modified feed 1418, in which the cells may be fed by using a flow rate into the IC inlet port (501A) of the bioreactor (501) and a flow rate into the IC outlet port (501B) of the bioreactor (501) to keep the cells in the bioreactor. In so doing, an inlet volumetric flow rate or inlet flow rate may be introduced into the first fluid flow path (506) 1420. For example, an IC inlet flow rate may be introduced into the first fluid flow path (506) 1420. The IC inlet pump (554), e.g., a first peristaltic pump (in an embodiment), may operate at a pre-defined number of revolutions per minute (RPMs) to cause a pre-defined IC inlet volumetric flow rate, or IC inlet flow rate, of fluid in the first fluid flow path (506) 1420. A processor(s) and/or controller(s) may direct or control the first pump and/or second pump, for example, to operate at a pre-defined number of RPMs, according to an embodiment. Depending on the speed and direction of the IC circulation pump (512), a modified first flow rate, or first portion of the IC inlet flow rate, may enter the IC inlet port (501A), or first port, of the bioreactor (501) 1422. A pump rate of a pump may depend on the diameter of the pump or configuration of the pump, e.g., peristaltic pump. Other types of pumps may also be used, in which the pump rate may depend on the configuration of the pump used. The IC circulation pump (512), e.g., a second peristaltic pump (in an embodiment), may operate at a pre-defined number of RPMs, and in a direction opposite a direction of the first pump, to cause or produce a pre-defined IC circulation flow rate, or second flow rate, or second portion of the IC inlet flow rate, to enter the IC outlet port (501B), or second port, of the bioreactor (501) 1424. For example, embodiments may provide for about one-half (about ½), or a first portion, of the IC inlet flow rate to branch at connection 517 to enter IC inlet port (501A) of bioreactor 501, and about one-half (or ½), or a second portion, of the IC inlet flow rate to branch at connection 517 to enter the IC outlet port (501B) of bioreactor 501. As shown, the sum of the first portion and the second portion may substantially equal the IC inlet flow rate 503 (e.g., FIG. 5B), in which the IC inlet flow rate 503 may be pumped by IC inlet pump 554. Depending on the flow and counter-flow rates which may be used to keep the cells in the bioreactor or cell growth chamber 501, other types of fractions or percentages of the IC inlet pump rate may be used to set, or configure or adjust, the IC circulation pump, according to embodiments.

In an example embodiment, the cells may be fed 916 through the EC circulation feed loop 752, the IC circulation feed loop 753, or a combination thereof, as described with reference to FIGS. 7A-7C.

After feeding the cells with such flow and counter-flow properties to keep the cells in the bioreactor, process 1400 proceeds to grow/expand the cells 1426. While the expansion of cells is shown at step 1426, the cells may also grow/expand during one or more other step(s), such as 1412, 1414, 1416, 1418, 1420, 1422, 1424, for example. From expand step 1426, process 1400 proceeds to harvest or remove the cells 1430. Process 1400 may then terminate at END operation 1432. If any other steps are desired before harvest, such as continuing with a second modified feed method or other type of feeding method, process 1400 proceeds to optional “Other” step 1428, according to embodiments. From optional step 1428, process 1400 proceeds to harvest or remove the cells from the bioreactor 1430, and process 1400 may then terminate at END operation 1432.

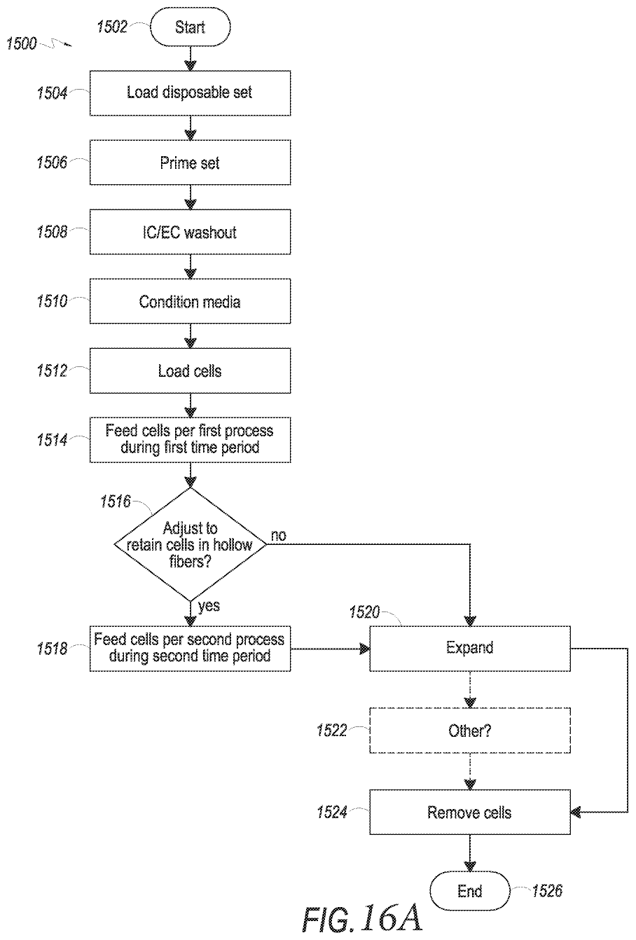

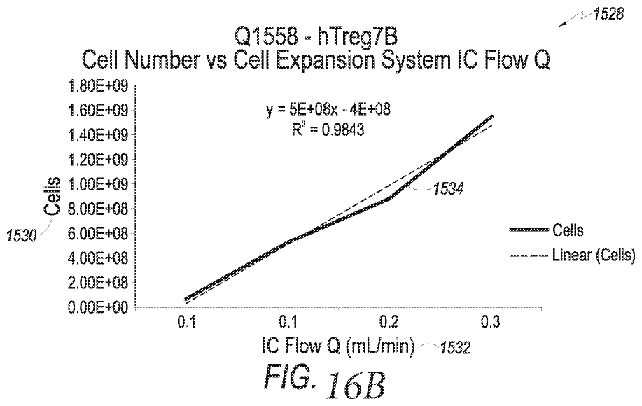

Turning to FIG. 16A, example operational steps of a process 1500 for feeding cells to retain the cells in a first location, e.g., in the bioreactor itself, that may be used with a cell expansion system, such as CES 500 (e.g., FIGS. 5B & 5C) or CES 700 (FIGS. 7A-7C), are provided in accordance with embodiments of the present disclosure. START operation 1502 is initiated, and process 1500 proceeds to load the disposable tubing set or premounted fluid conveyance assembly (e.g., 210 or 400) 1504 onto the cell expansion system. The disposable set may include the cell growth chamber 100A or the cell growth chamber 100B, as illustrated in FIGS. 1C, 4B, and 4C. Next, the system may be primed 1506. In an embodiment, a user or operator, for example, may provide an instruction to the system to prime by selecting a task for priming, for example. In an embodiment, such task for priming may be a pre-programmed task. Next, an IC/EC washout task may be performed 1508, in which fluid on the IC circulation loop and on the EC circulation loop may be replaced, for example. The replacement volume may be determined by the number of IC volumes and EC volumes exchanged, according to an embodiment.

Next, to maintain the proper or desired gas concentration across the fibers in the bioreactor membrane, the condition media task 1510 may be executed to allow the media to reach equilibrium with the provided gas supply before cells are loaded into the bioreactor. For example, contact between the media and the gas supply provided by the gas transfer module (GTM) or oxygenator may be provided by adjusting the EC circulation rate. The system may then be maintained in a proper or desired state until a user or operator, for example, is ready to load cells into the bioreactor. In an embodiment, the system may be conditioned with media, such as complete media, for example. Complete media may be any media source used for cell growth. In an embodiment, the system may be conditioned with serum-free media, for example. In an embodiment, the system may be conditioned with base media. Any type of media understood by those of skill in the art may be used.

Process 1500 next proceeds to loading cells 1512 into the bioreactor from a cell inlet bag, for example. In an embodiment, the cells in the cell inlet bag may be in solution with media to feed the cells, for example. In another embodiment, the cells in the cell inlet bag may be in solution both with media and with a soluble activator complex to stimulate the cells, e.g., T cells or Tregs. In an embodiment, the cells may be loaded into the bioreactor from the cell inlet bag until the bag is empty. Cells may be chased from the air removal chamber to the bioreactor. In an embodiment, a “load cells with uniform suspension” task may be executed to load the cells. In another embodiment, a “load cells centrally without circulation” task may be executed to load the cells into a specific, e.g., central, region of the bioreactor. Other loading methods and/or loading tasks may be used in accordance with embodiments.

Next, process 1500 proceeds to feed the cells per a first process during a first time period 1514. In an example embodiment, the cells may be fed 916 through the EC circulation feed loop 752, the IC circulation feed loop 753, or a combination thereof, as described with reference to FIGS. 7A-7C. In an embodiment, the cells may be fed at a minimum or low feed rate, for example, where the cell population is beginning to grow/expand, and a minimum or low feed rate is able to meet the feeding demands of such population. For example, an IC inlet pump rate of +0.1 mL/min to cause or produce a first fluid flow rate of 0.1 mL/min may be used during such first time period. While this example provides for a low or minimum feed rate of 0.1 mL/min, a low or minimum feed rate may be greater than or equal to about 0.01 mL/min and less than or equal to about 0.1 mL/min, according to embodiments. In embodiments, the low or minimum feed rate may be greater than 0.1 mL/min. If it is desired to reduce the loss of cells from the hollow fiber membrane bioreactor during such first time period, the IC inlet pump rate of +0.1 mL/min may be matched, or closely or substantially matched, to a complementary IC circulation pump rate of −0.1 mL/min to maintain cells in the bioreactor during the growth phase of the cell culture. Other pump rates and resulting fluid flow rates may be used in other embodiments. Alternatively, in an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture.

Next, process 1500 proceeds to query 1516 to determine whether to adjust the feeding rate to retain the cells in the bioreactor itself while also accounting for a growing cell population and increasing feeding demands, according to embodiments. For example, FIG. 16B shows an increasing feed rate in response to an increasing cell population. As shown in FIG. 16B, graph 1528 shows the cell number versus the IC flow rate for a run or procedure on a cell expansion system, e.g., Quantum® Cell Expansion System. In an embodiment, the IC flow rate comprises media for feeding the cells, and may thus also be referred to as the IC media flow rate. The number of cells 1530 is shown versus the IC flow rate (mL/min) 1532. As shown, the IC flow rate increases from 0.1 mL/min to 0.2 mL/min to 0.3 mL/min with an increase in the cells and the growing feeding demands of an expanding cell population, according to an embodiment. In the embodiment shown, there may be a substantially linear relationship, as shown by line 1534, between the number of cells and the IC flow rate.

Returning to FIG. 16A and query 1516, if it is not desired to adjust the feeding rate, process 1500 proceeds “no” to expand the cells 1520, in which the cells may continue to grow/expand using the media with which they were fed during the first time period 1514, for example. While the expansion of cells is shown at step 1520, the cells may also grow/expand during one or more other step(s), such as 1512, 1514, 1516, 1518, for example. On the other hand, if it is desired to adjust the feeding rate while keeping the cells in the bioreactor, process 1500 proceeds “yes” to feed the cells per a second process during a second time period 1518. In an embodiment, such second process may involve feeding the cells at substantially the same feed rates as during the first time period, for example. In another embodiment, the second process may involve feeding the cells at different feed rates as compared to the feed rates used during the first time period. In an embodiment, the IC inlet flow rate may be increased, and the IC circulation flow rate may be set, or configured or adjusted, to equal a percentage or portion or fraction of the IC inlet flow rate. For example, the IC circulation flow rate may be set to equal about fifty percent (50%) or about one-half (½), or another percentage or portion according to embodiments, of the value of the IC inlet flow rate, according to an embodiment. Determining whether to set, configure or adjust, the IC circulation flow rate to a percentage or fraction or portion of the IC inlet flow rate may be based on the value of the IC inlet flow rate, according to an embodiment. For example, an embodiment provides for the following method to retain cells in a bioreactor when feeding the cells using IC inlet flow (where Q_{IC circ}=IC circulation flow rate (mL/min); Q_{IC Inlet}=IC Inlet flow rate (mL/min)):

Q_{IC Circ}=(−)½*Q_{IC Inlet} when Q_{IC Inlet}≥0.2 mL/min

and

Q_{IC Circ}=(−)Q_{IC Inlet} when Q_{IC Inlet}=0.1 mL/min.

While the above equations provide for different calculations of the IC circulation flow rate based on the values of the IC inlet flow rates (e.g., 0.2 mL/min or 0.1 mL/min), other values of such IC inlet flow rate for making such determination may be used, according to other embodiments. Further, while about fifty percent (50%) or about one-half (½) is used in this example, other percentages, ratios, fractions, and/or portions may be used in accordance with embodiments. Returning to process 1500, after feeding the cells with such flow and counter-flow properties as a part of second process 1518 to keep the cells in the bioreactor, process 1500 proceeds to grow/expand the cells 1520. While the expansion of cells is shown at step 1520, the cells may also grow/expand during one or more other step(s), such as 1512, 1514, 1516, 1518, for example. From expand step 1520, process 1500 proceeds to harvest or remove the cells 1524. Process 1500 may then terminate at END operation 1526. If any other steps are desired before harvesting the cells, process 1500 proceeds to optional “Other” step 1522, according to embodiments. From optional step 1522, process 1500 proceeds to harvest or remove the cells from the bioreactor 1524, and process 1500 may then terminate at END operation 1526.

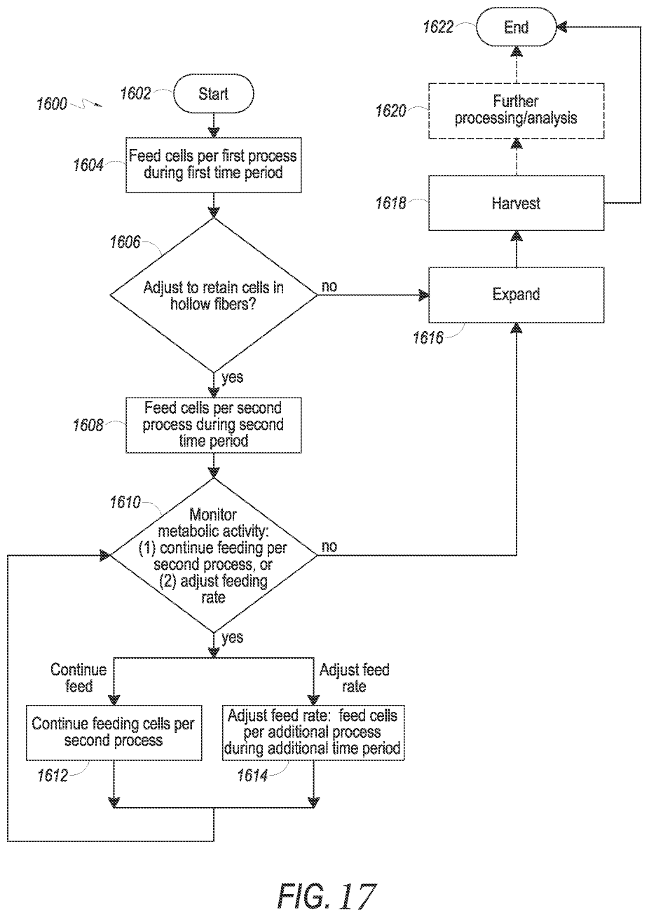

Next, FIG. 17 illustrates example operational steps of a process 1600 for feeding cells that may be used with a cell expansion system, such as CES 500 (e.g., FIGS. 5B & 5C) or CES 700 (FIGS. 7A-7C), in accordance with embodiments of the present disclosure. START operation 1602 is initiated, in which a disposable set may be loaded onto a cell expansion system, the system may be primed, IC/EC washout may be performed, media may be conditioned, and cells may be loaded, for example. The disposable set may include the cell growth chamber 100A or the cell growth chamber 100B, as illustrated in FIGS. 1C, 4B, and 4C. Process 1600 next proceeds to feed the cells according to a first process during a first time period 1604. In an example embodiment, the cells may be fed 916 through the EC circulation feed loop 752, the IC circulation feed loop 753, or a combination thereof, as described with reference to FIGS. 7A-7C. In an embodiment, the cells may be fed at a minimum or low feed rate, for example, where the cell population is beginning to grow/expand, and a minimum or low feed rate is able to meet the feeding demands of such population. For example, an IC inlet pump rate of +0.1 mL/min may be used during such first time period. While this example provides for a low or minimum feed rate of 0.1 mL/min, a low or minimum feed rate may be greater than or equal to about 0.01 mL/min and less than or equal to about 0.1 mL/min, according to embodiments. In embodiments, the low or minimum feed rate may be greater than 0.1 mL/min. If it is desired to reduce the loss of cells from the hollow fiber membrane bioreactor during such first time period, the IC inlet pump rate of +0.1 mL/min may be matched, or closely or substantially matched, to a complementary IC circulation pump rate of −0.1 mL/min to maintain cells in the bioreactor during the growth phase of the cell culture. Alternatively, in an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture.

Next, process 1600 proceeds to query 1606 to determine whether the feeding rate may be adjusted to account for a growing cell population and/or to continue efforts to keep the cells in the bioreactor. If it is desired to adjust the feeding rate to retain the cells in the bioreactor, process 1600 proceeds “yes” to feed the cells according to a second process during a second time period 1608. In an embodiment, such second process may involve feeding the cells at substantially the same feed rates as during the first time period, for example. In another embodiment, the second process may involve feeding the cells at different feed rates as compared to the feed rates used during the first time period. In an embodiment, the IC inlet flow rate may be increased, and the IC circulation flow rate may be set, or configured or adjusted, to equal a percentage or fraction or portion of the IC inlet flow rate. For example, the IC circulation flow rate may be set to equal about fifty percent (50%) or about one-half (½), or another percentage or portion according to embodiments, of the value of the IC inlet flow rate, according to an embodiment. Determining whether to set the IC circulation flow rate to a percentage of the IC inlet flow rate may be based on the value of the IC inlet flow rate, according to an embodiment. For example, an embodiment provides for the following (where Q_{IC Circ}=IC circulation flow rate (mL/min), Q_{IC Inlet}=IC Inlet flow rate (mL/min)):

Q_{IC Circ}=(−)½*Q_{IC Inlet} when Q_{IC Inlet}≥0.2 mL/min

and

Q_{IC Circ}=(−)Q_{IC Inlet} when Q_{IC Inlet}=0.1 mL/min.

While the above equations provide for different calculations of the IC circulation flow rate based on the values of the IC inlet flow rates (e.g., 0.2 mL/min or 0.1 mL/min), other values of such IC inlet flow rate for making such determination may be used, according to other embodiments. Further, while about fifty percent (50%) or about one-half (½) is used in this example, other percentages, ratios, fractions, and/or portions may be used in accordance with embodiments. Returning to process 1600, after feeding the cells with such flow and counter-flow properties as a part of second process 1608 to keep the cells in the bioreactor, process 1600 proceeds to query 1610 to determine whether to monitor or measure the metabolic activity, e.g., glucose consumption and/or lactate generation, of the growing cell population. If it is not desired to monitor the metabolic activity, process 1600 proceeds “no” to expand the cells 1616, in which the cells may continue to grow/expand using the media with which they were fed during the first time period 1604 and/or second time period 1608, for example. While the expansion of cells is shown at step 1616, the cells may also grow/expand during one or more other step(s), such as 1604, 1606, 1608, 1610, 1612, 1614, for example.

Returning to query 1610, if it is desired to monitor or measure the metabolic activity of the growing cell population, process 1600 proceeds “yes” to either continue to feed the cells per the second process or adjust the feed rate, based on the metabolic activity and/or measurements thereof. In an embodiment, monitoring the glucose and/or lactate levels may facilitate the adjustment of media flow rates, e.g., IC flow rate, to support cell, e.g., T cell or Treg, expansion in a bioreactor, e.g., hollow fiber bioreactor. In embodiments, cell culture lactate values may be maintained below about 7 mmol/L, for example. In embodiments, by using a cell expansion system graphical user interface (GUI), for example, to control a rate(s) of media addition, lactate metabolic waste product from glycolysis may be maintained below about 7 mmol/L, for example, during the expansion of cells, e.g., regulatory T cells. In other embodiments, rate(s) of media addition, for example, and/or other settings may be controlled to attempt to maintain the lactate levels≤about 5 mmol/L, for example, to improve cell growth and viability. Other concentrations may be used in other embodiments.

Depending on the metabolic measurements and the desired levels of lactate, for example, process 1600 proceeds to either continue feeding the cells according to the second process 1612 or adjusting the feed rate 1614. For example, the cells may be continued to be fed according to the second process 1612 where measurements of the metabolic activity show lactate levels≤about 5 mmol/L, according to an embodiment. In another embodiment, the cells may be continued to be fed according to the second process 1612 where the measurements of the metabolic activity show lactate levels≤about 7 mmol/L, for example From continuing to feed the cells per the second process 1612, process 1600 returns to query 1610 to continue monitoring the metabolic activity of the growing cell population.

Depending on the metabolic measurements and the desired levels thereof, process 1600 proceeds to adjust the feed rate 1614, in which the cells may be fed according to an additional process during an additional time period. Such additional process(es) and additional time period(s) may include, for example, a third process during a third time period, a fourth process during a fourth time period, a fifth process during a fifth time period, etc., according to embodiments. In an embodiment, such additional process may involve feeding the cells at substantially the same feed rates as during the first and/or second time periods, for example. In another embodiment, the additional process may involve feeding the cells at different feed rates as compared to the feed rates used during the first and/or second time periods. For example, the IC inlet flow rate may be increased, and the IC circulation flow rate may be matched, or closely or substantially matched, to the IC inlet flow rate, but in the opposite direction, in an embodiment. In another embodiment, the IC inlet flow rate may be increased, and the IC circulation flow rate may be set, or configured or adjusted, to equal a percentage or fraction or portion of the IC inlet flow rate, and in the opposite direction. While adjusting the feed rate 1614 shows an “additional” process and an “additional” time period in step 1614, any number of processes and time periods may be used to adjust the feed rate based on metabolic activity.

From adjusting the feed rate 1614, process 1600 returns to query 1610. If it is not desired to adjust, or further adjust, the feeding rate, process 1600 proceeds “no” to expand the cells 1616, in which the cells may continue to grow/expand using the media with which they were fed during the first time period 1604, second time period 1608, and/or additional time period 1614. While the expansion of cells is shown at step 1616, the cells may also grow/expand during one or more other step(s), such as step(s) 1604, 1606, 1608, 1610, 1612, 1614, for example. From expand step 1616, process 1600 proceeds to harvest or remove the cells 1618 from the bioreactor and into a harvest bag(s) or container(s), for example. Process 1600 may then terminate at END operation 1622. Alternatively, from harvest operation 1618, process 1600 may optionally proceed to allow for further processing/analysis 1620. Such optional further processing/analysis 1620 may include characterizing the phenotype(s), for example, of the harvested cells, e.g., T cells or Tregs. From optional further processing/analysis step 1620, process 1600 may then terminate at END operation 1622.

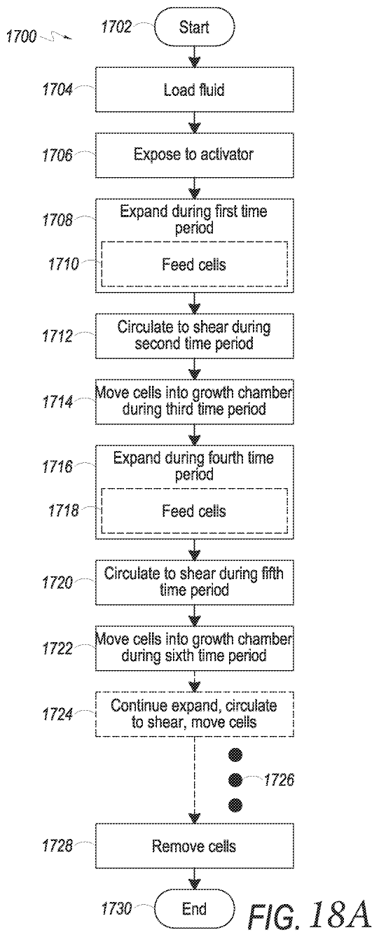

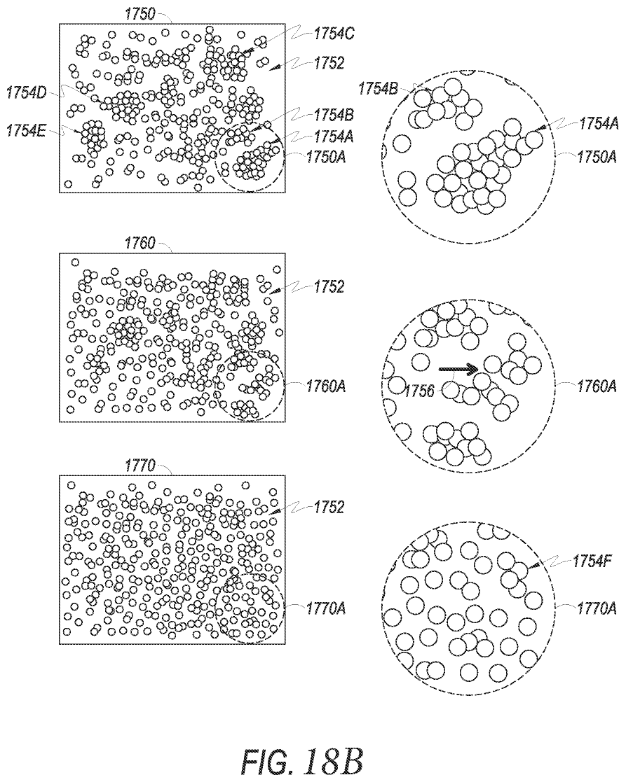

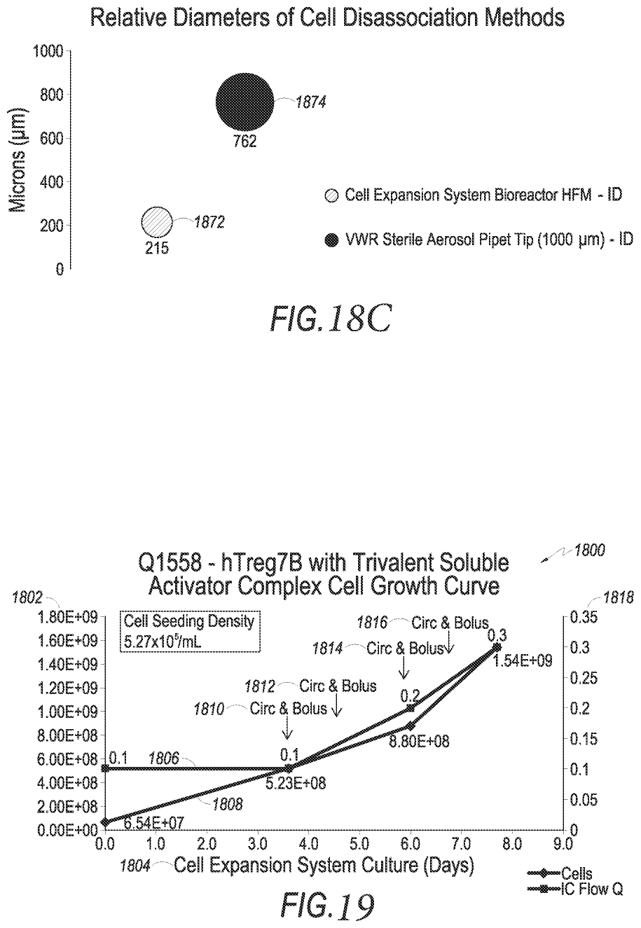

FIG. 18A illustrates operational steps of a process 1700 for expanding cells that may be used with a cell expansion system in embodiments of the present disclosure. As described below, process 1700 may include steps to shear cells that have been expanded in the cell growth chamber according to embodiments of the present disclosure. In embodiments, these steps may be implemented as part of a “modified circulation” task. START operation 1702 is initiated and process 1700 proceeds to loading fluid 1704 with cells into a cell growth chamber in a cell expansion system. In embodiments, the cells may comprise non-adherent cells, such as one or more types of T cells. In one embodiment, the cells include Tregs.

Process 1700 proceeds to exposing the cells to an activator 1706. The activator, which may include antibody complexes, may be added to the fluid loaded at step 1704. In embodiments, the activator may be a soluble human antibody CD3/CD28/CD2 cell activator complex. Process 1700 proceeds to expanding 1708 the cells for a first time period. Step 1708 may include feeding 1710 the cells. The cells may be fed nutrients to promote their expansion. For example, media with glucose, proteins, and reagents may be delivered into the cell growth chamber to provide nutrients for cell expansion.

The first time period for expanding 1708 the cells may be based on the time it may take for cell colonies, micro-colonies, or clusters to form. A cell colony, micro-colony, or cluster may be a group of one or more attached cells. In embodiments, the cells, e.g., Tregs, may benefit from cell contact. The cell contact may stimulate signaling that promotes expansion and growth. However, after a period of expansion, the cells may attach to each other to form cell colonies, micro-colonies, or clusters. Without being bound by theory, it is believed that after a time period of the cells expanding 1708 the cells may form relatively large cell colonies, micro-colonies, or clusters that continue to grow. The cell colonies, micro-colonies, or clusters may create necrotic centers where nutrients (e.g., glucose), gasses (e.g., oxygen), and reagents (e.g., activator) do not reach cells in the center of the cell colonies, micro-colonies, or clusters. As a result, the conditions for cell expansion in the center of these cell colonies, micro-colonies, or clusters may be such that the expansion rate may slow (e.g., increase doubling time) or the conditions may lead to cell necrosis.

In embodiments, to allow the expansion 1708 of the cells, the first time period may be between about 5 hours and about 48 hours. In some embodiments, the first time period may be greater than about 6 hours, greater than about 12 hours, greater than about 24 hours, or even greater than about 48 hours. In other embodiments, the first time period may be less than about 72 hours, less than about 60 hours, less than about 48 hours, less than about 36 hours, less than about 24 hours, or even less than about 12 hours. After the first time period, process 1700 proceeds to circulate 1712 to shear cell colonies, micro-colonies, or clusters during a second time period. Step 1712 may be performed to reduce the size of the cell colonies, micro-colonies, or clusters. The second time period may in embodiments be less than about 120 minutes such as between about 60 minutes and about 0.5 minutes. In other embodiments, the second time period may be based on a volume of fluid introduced into the first circulation path.

FIG. 18B illustrates a number of views 1750, 1760, and 1770 of cells in a volume of fluid (1752) that may be expanded in a cell growth chamber as part of process 1700. For example, in some embodiments, the cell growth chamber may be a hollow fiber bioreactor. In these embodiments, views 1750, 1760, and 1770 may illustrate cells in a fiber of a hollow fiber bioreactor, for example. 1750A, 1760A, and 1770A are zoomed in portions of views 1750, 1760, and 1770, respectively. Referring to view 1750, the cells may be shown after cells have been loaded 1704, exposed to an activator, 1706 and expanded 1710 for a time period, e.g., the first time period. As illustrated in view 1750, a number of cell colonies 1754A-E have formed.

In order to reduce the number of cells in, and the size of, the cell colonies, micro-colonies, or clusters 1754A-E, step 1712 may circulate fluid and the cells through a first fluid circulation path. Without being bound by theory, it is believed that the circulation may create some force acting on the cell colonies including shear stress, as illustrated by arrow 1756 as shown in zoomed in portion 1760A. The shear stress 1756 may provide enough force to separate cells in the cell colonies. As the circulation continues, the cell colonies may begin to break up into smaller sizes, as shown in view 1760. View 1770 illustrates the cells after the circulation has been performed for the second time period. As illustrated in view 1770, the size of the cell colonies are reduced with some colonies being completely separated into individual cells. In some embodiments, the circulation to shear step 1712 may be performed until the cells and fluid comprise a single cell suspension.

In other embodiments, cell colonies, micro-colonies, or clusters of cells may remain after circulate to shear 1712. For example, colony 1754F in zoomed in portion 1770A illustrates that some colonies of a reduced size may remain after step 1712. In embodiments the cell colonies, micro-colonies, or clusters (e.g., 1754F) that remain may be between about 25 microns and about 300 microns. In other embodiments, circulate to shear 1812 may reduce the size of cell colonies, micro-colonies, or clusters (e.g., 1754F) so the cell colonies, micro-colonies, or clusters may be between about 50 microns and about 250 microns. In yet other embodiments, step 1712 may reduce the size of cell colonies, micro-colonies, or clusters to between about 75 microns and about 200 microns. In some embodiments, the size of the cell colonies, micro-colonies, or clusters may be less than about 200 microns e.g., about 100 microns after step 1712.

In embodiments, the size of the remaining cell colonies, micro-colonies, or clusters may be somewhat a function of some structural features of the cell growth chamber. As mentioned above, the cell growth chamber may be a hollow fiber bioreactor with hollow fibers in some embodiments. As may be appreciated, cell colonies, micro-colonies, or clusters, as they circulate, may be affected by shear stress each time they contact the side walls of the hollow fiber. This contact may more efficiently reduce the size of cell colonies. When the inner diameter is larger, such as in a conventional process that may utilize a pipet to induce shear stress to reduce colony sizes, contact with a side wall may not occur as often. FIG. 18C illustrates differences in inner diameter size between one embodiment of a hollow fiber (e.g., 215 microns) 1772 and a pipet tip 1774 (762 microns), which may be used to disassociate attached cells in cell colonies, micro-colonies, or clusters. In embodiments, the smaller inner diameter of a hollow fiber is believed to more efficiently and effectively reduce a size of cell colonies during the circulating to shear step 1712.

The second time period for the circulate to shear 1712 may in embodiments be less than about 120 minutes, less than about 90 minutes, less than about 60 minutes, less than about 30 minutes, or even less than about 15 minutes. In some embodiments, the second time period may be between about 15 minutes and about 1 minute, such as about 4 minutes.

After the second time period, process 1700 proceeds to move cells into cell growth chamber during a third time period 1714. At step 1714 cells that are not positioned in the cell growth chamber, as a result of the circulation to shear step 1712, are moved back into the cell growth chamber during a third time period. In embodiments, this may involve activating one or more pumps to introduce fluid into a fluid circulation path. For example, fluid may be introduced from a fluid inlet path to a first fluid flow path and then into the cell growth chamber, from both an inlet port and an outlet port of the cell growth chamber. The movement of the fluid into the cell growth chamber from the inlet port and the outlet port may move cells back into the cell growth chamber.

In some embodiments, the fluid used in the step to move the cells back into the cell growth chamber at 1714 may include reagents that promote cell growth. For example, in embodiments, the fluid may be media that includes glucose, proteins, or other reagents. In one embodiment, the fluid may include one or more supplements. In one embodiment, the fluid is complete media and includes a cytokine, e.g., human IL-2 cytokine supplement. The addition of the fluid may be referred to as a bolus addition. The combination of steps 1712 and 1714 may be referred to in embodiments as circulate and bolus addition.

In other embodiments, the third time period may be based on a volume of fluid introduced into the cell growth chamber during the circulate to shear step 1712. For example, in embodiments, step 1712 may be performed until about 300 ml, about 250 ml, about 200 ml, or about 150 ml, have been introduced into the fluid circulation path.

After the third time period, process 1700 proceeds to expand during a fourth time period 1716. Similar to step 1708, step 1716 may include feeding 1718 the cells. The cells may be fed nutrients to promote their expansion. For example, media with glucose, proteins, and reagents may be delivered into the cell growth chamber to provide nutrients for cell expansion.

Similar to the first time period, the fourth time period may be based on the time it may take for cell colonies to form. In embodiments, the fourth time period may be between about 5 hours and about 48 hours. In some embodiments, the fourth time period may be greater than about 6 hours, greater than about 12 hours, greater than about 24 hours, or even greater than about 48 hours. In other embodiments, the fourth time period may be less than about 72 hours, less than about 60 hours, less than about 48 hours, less than about 36 hours, less than about 24 hours, or even less than about 12 hours. Because more cells are likely in the cell growth chamber, the fourth time period may be shorter than the first time period in some embodiments.

After the fourth time period, process 1700 proceeds to step 1720 to circulate to shear for a fifth time period to reduce second cell colonies. In embodiments, step 1720 may use the first circulation rate. However, in other embodiments, the circulation rate used at step 1720 may be different, either greater or less than the first circulation rate.

After the fifth time period, process 1700 proceeds to step 1722 where the cells that are not positioned in the cell growth chamber, may be moved back into the cell growth chamber during a sixth time period. Fluid may be introduced from a fluid inlet path to a first fluid flow path and into the cell growth chamber from an inlet port and an outlet port of the cell growth chamber. The movement of the fluid into the cell growth chamber from the inlet port and the outlet port may move cells back into the cell growth chamber. In some embodiments, the fluid used to move the cells back into the cell growth chamber may include reagents that promote cell growth. For example, in embodiments, the fluid may be media that includes glucose, proteins, or other reagents. In one embodiment, the fluid may include one or more supplements. In one embodiment, the fluid is complete media and includes a cytokine, e.g., human IL-2 cytokine.

The process 1700 may optionally perform the steps of expand, circulate, and move cells for an additional number of times as illustrated by optional step 1724 and ellipsis 1726. The steps of expand, circulate, and move may be performed sequentially for a period of time. For example, in some embodiments, the steps may be performed once every four days, once every three days, once every two days, daily, twice daily, or three times daily, for a period of from about two days to about twenty days (such as about 10 days). In some embodiments, the steps may be performed at varying periods of time. For example, in one embodiment, the steps may be performed after three days, and then every other day. As another example, the steps may be performed after two days and then twice daily. These are merely examples and other embodiments may utilize other periods of time.

For example, FIG. 19 shows a graph 1800 of performing circulation and bolus additions at different periods of time during a process of expanding cells. Curve 1808 shows the cell number 1802 versus days of cell culture 1804 on a cell expansion system, e.g., Quantum® Cell Expansion System. Curve 1806 shows IC Flow rate 1818 versus days of cell culture 1804 on a cell expansion system, e.g., Quantum® Cell Expansion System. As shown by curve 1806, the IC flow rate remains the same at 0.1 mL/min for the first three days. There is an increase in the flow rate to 0.2 mL/min at day six and an increase to 0.3 mL/min after day seven. The increase in flow rate may be in response to the increase in cell numbers as the culture days 1804 increase. Also, shown in FIG. 19, are several circulation and bolus addition steps (e.g., 1810, 1812, 1814, and 1816). There is a circulation and bolus addition 1810 performed 3.5 days after cell culture. There is another circulation and bolus addition 1812 performed after 4.5 days of cell culture. Another circulation and bolus addition is performed after 6 days of cell culture 1814, and another circulation and bolus addition after 6.5 days of cell culture 1816. As may be appreciated by looking at curves 1806 and 1808, the multiple circulation and bolus additions (1810-1816), in combination with the increasing IC flow rates, may have a positive effect on the rate of cell expansion, e.g., number of cells.

Referring back to 18A, process 1700 proceeds to remove cells 1728 from the cell growth chamber. In embodiments, this may involve harvesting the cells. Step 1728 may include additional steps such as circulation steps (e.g., 1712 and 1720) prior, or during, removal of the cells from the cell growth chamber. Process 1700 terminates at END operation 1730.

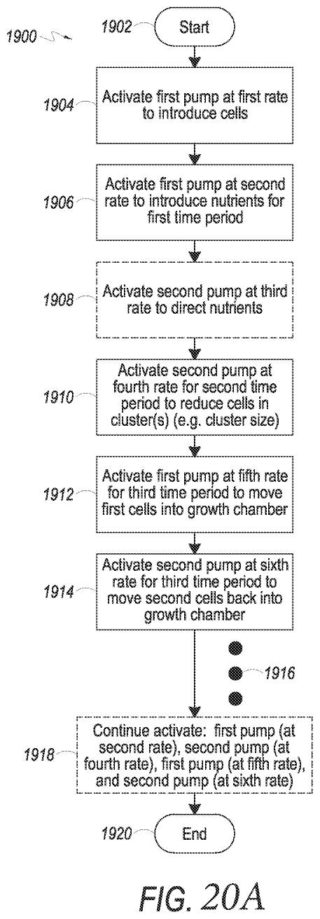

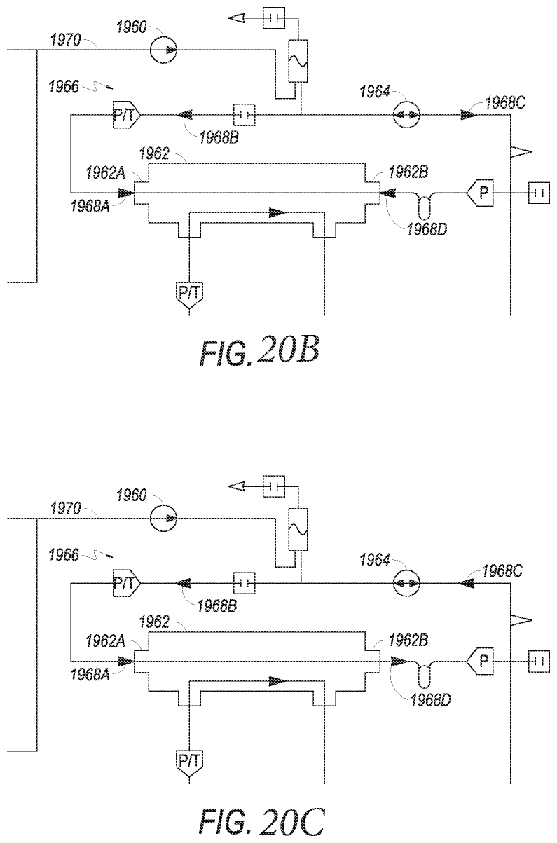

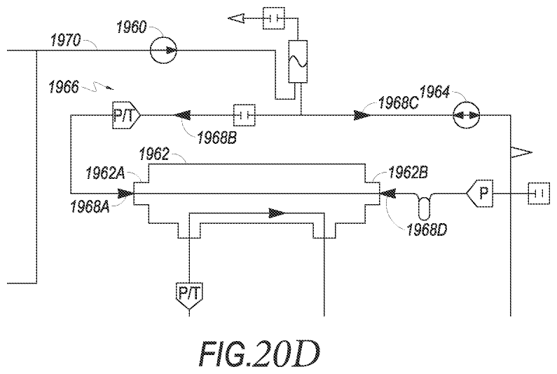

FIG. 20A illustrates operational steps of a process 1900 for operating pumps that may be used in a cell expansion system in embodiments of the present disclosure. As described below, process 1900 may include steps to activate pumps in a process of reducing cells in cell clusters that have been expanded in the cell growth chamber according to embodiments of the present disclosure. In embodiments, these steps may be implemented as part of a “modified circulation” task. In embodiments, the steps of process 1900 may be performed by a computer processor. START operation 1902 is initiated and process 1900 proceeds to step 1904, where a first pump is activated at a first flow rate to introduce a first volume of fluid including cells into the intracapillary portion of a bioreactor of a cell expansion system. For example, the bioreactor may be the cell growth chamber 100A or the cell growth chamber 100B, as illustrated in FIGS. 1C, 4B, and 4C. In embodiments, the first pump may be an inlet pump.

After activating the first pump at the first flow rate, process 1900 proceeds to the first pump activated at a second flow rate 1906 to introduce media with nutrients into the intracapillary portion of the bioreactor for a first time period. The nutrients may include for example proteins, glucose, and other compounds that are used to feed and promote the expansion of the cells. For example, referring to FIG. 20B, a first pump 1960 may be activated at the second flow rate to introduce media into an inlet port 1962A of bioreactor 1962.

In embodiments, the first time period may be based on the time it may take for cell colonies to form. In embodiments, the first time period may be between about 5 hours and about 48 hours. In some embodiments, the first time period may be greater than about 6 hours, greater than about 12 hours, greater than about 24 hours, or even greater than about 48 hours. In other embodiments, the first time period may be less than about 72 hours, less than about 60 hours, less than about 48 hours, less than about 36 hours, less than about 24 hours, or even less than about 12 hours.

Process 1900 then proceeds to activating a second pump at a third flow rate to direct fluid into the bioreactor at 1908. Optional step 1908 may be performed to activate pump 1964 to direct a portion of the fluid, introduced at step 1906, into an outlet port 1962B of the bioreactor 1962 to feed cells, for example.

Process 1900 then proceeds to activating the second pump at a fourth flow rate 1910 to circulate the cells during a second time period and reduce a number of cells in a cell cluster in the bioreactor. In embodiments, the cells are circulated throughout a first fluid circulation path. Referring to FIG. 20C, second pump 1964 may be activated at step 1910 to circulate fluid through first fluid circulation path 1966, is illustrated by arrows 1968A-D.

Without being bound by theory, it is believed that after a time period the expanding cells may form cell colonies, micro-colonies, or clusters. The cell colonies may create necrotic centers where nutrients and proteins (e.g., activator) do not reach cells in the center of the colonies. As a result, the conditions for cell expansion in the center of these cell colonies, micro-colonies, or clusters may be such that the expansion rate may slow (e.g., increase doubling time) and may result in cell necrosis. Step 1910 may be performed to reduce the size of the cell colonies, micro-colonies, or clusters.

In embodiments, the fourth flow rate may be high enough to induce shearing. For example, in embodiments, the fourth flow rate may be as high as about 1000 ml/min. In other embodiments, the fourth flow rate may be between about 100 mL/min and about 600 ml/min, such as for example, 300 mL/min.

After the second time period, process 1900 proceeds to step 1912, where the first pump is activated at a fifth flow rate to introduce fluid through a first fluid flow path for a third time period. A first portion of the fluid introduced in the first fluid flow path may in embodiments move first cells in the first fluid flow path (that may be in the first fluid flow path because of step 1910) back into the cell growth chamber through the inlet port 1962A. Referring to FIG. 20D, pump 1960 may be activated to introduce fluid into the first fluid flow path 1970. As illustrated by arrow 1968A and 1968B, fluid flows from the first fluid flow path 1970 into inlet port 1962A. This moves first cells that are outside bioreactor 1962 back into bioreactor 1962. It is believed that moving cells that are in the first fluid flow path back into the cell growth chamber improves the overall expansion of cells, since the conditions for cell growth are optimized in the cell growth chamber.

In some embodiments, the fluid introduced into the first fluid flow path at step 1912, may include one or more materials (e.g., reagents) that promote cell expansion. For example, in embodiments, the fluid may be media that includes glucose or other nutrients for feeding the cells. In one embodiment, the fluid may include a reagent, which may comprise additional activator for continuing to activate the expansion of cells. The use of the fluid with particular reagent(s) or other materials to move the cells back into the cell growth chamber, and also expose the cells to additional reagents, e.g., growth factors, proteins, etc., that promote expansion may provide improved cell expansion. In embodiments, the additional fluid used in step 1912 may be referred to as a bolus addition.

Also, after the third time period, at step 1914, the second pump may be activated at a sixth flow rate to move a second portion of the fluid introduced through the first fluid flow path, and second cells, into the cell growth chamber through the outlet port 1962B. The second portion of the fluid may in embodiments move second cells in the first fluid flow path (that may be in the first fluid flow path because of step 1910) back into the cell growth chamber through the outlet port. Referring to FIG. 20D, pump 1964 may be activated to move fluid introduced into the first fluid flow path 1970 to the outlet port 1962B. As illustrated by arrow 1968C, pump 1964 moves fluid and cells in first fluid circulation path into bioreactor 1962 through outlet port 1962B. As illustrated, by FIG. 20D, the sixth flow rate may be in a direction opposite the fourth flow rate (FIG. 20C). This fluid movement moves cells that are outside bioreactor 1962 back into bioreactor 1962.

In embodiments, the sixth flow rate may be less than the fifth flow rate, which as noted above moves fluid into the first fluid flow path. As may be appreciated, the fifth flow rate may result in introduction of a volume of fluid into a portion of the circulation path 1966 based on the fifth flow rate. The sixth rate may be set so that a percentage of that volume moves toward the outlet port 1962B.

In embodiments, the sixth flow rate may be set as a percentage of the fifth flow rate. For example, the sixth flow rate may be less than or equal to about 90% of the fifth flow rate. In some embodiments, the sixth flow rate may be set to less than or equal to about 80% of the fifth flow rate. In other embodiments, the sixth flow rate may be less than or equal to about 70% of the fifth flow rate. In yet other embodiments, the sixth flow rate may be less than or equal to about 60% of the fifth flow rate. In some embodiments, the sixth flow rate may be less than or equal to about 50% of the fifth flow rate.

In embodiments, the sixth flow rate is based, at least in part, on the difference between a first volume, between the second pump and the inlet port, and a second volume, between the second pump and the outlet port. Referring to FIG. 20D, portions of the first circulation path may have different volumes. For example, in one embodiment, a first volume between the second pump 1964 and the inlet port 1962A may have a first volume, and a second volume between the second pump 1964 and the outlet port 1962B may have a second volume that is different from the first, e.g., larger. In these embodiments, in order to move the cells from the circulation path back into the bioreactor, so that the cells generally reach the bioreactor at approximately the same time, the sixth flow rate may be set at least in part based on the differences in these volumes.

As may be appreciated, as fluid enters fluid circulation path 1966 from first fluid flow path 1970, the fluid moves toward inlet port 1962A at the flow rate that first pump 1960 is set. When pump 1964 is activated, it will redirect at least a portion of the fluid toward the outlet port 1962B. In embodiments, the second volume (the volume between 1964 and outlet port 1962B) may be larger than the first volume. Therefore, in order to move more fluid into the second volume, the second pump 1964 may be set at a percentage of the rate of pump 1960.

In one embodiment, at step 1912, pump 1960 may be set to 100 ml/min. In this embodiment, the second volume (from pump 1964 to outlet port 1962B) may be larger than the first volume (from pump 1964 to inlet port 1962A). In order to account for the additional volume, embodiments may provide for pump 1964 to be set at 70 ml/min during step 1914. This embodiment may provide for cells to reach bioreactor 1962 during the third time period at about the same time.

In embodiments, process 1900 may optionally perform the steps of 1906-1914 a number of additional times as illustrated by ellipsis 1916 and optional step 1918. The steps of Activate: First Pump (at Second Rate) 1906, Second Pump (at Fourth Rate) 1910, First Pump (at Fifth Rate) 1912, and Second Pump (at Sixth Rate) 1914 may be continuously performed for a period of time. As noted above, the steps may be performed to feed cells, circulate cells to break up cell colonies, micro-colonies, or clusters, and move cells back into a cell growth chamber. For example, in some embodiments, the steps may be performed every three days, every two days, daily, twice daily, or three times a day, for a period of from about two days to about twenty days (such as 10 days). In some embodiments, the steps may be performed at varying periods of time. For example, in one embodiment, the steps may be performed after three days, and then every other day. As another example, the steps may be performed after two days and then twice daily. This is merely one example and other embodiments may utilize other periods of time. Process 1900 terminates at END operation 1920.

It is noted that in some embodiments, process 1900 may include additional steps. For example, a rocking device may be connected to the bioreactor and after the first time period (and during the second time period), when the first pump is activated at step 1910, the rocking device may be activated to rotate the bioreactor as part of circulating the cells to reduce a number of cells in a cell cluster. This is merely one example and other embodiments of process 1900 are not limited thereto.

In an alternative embodiment, the steps of process 1900 may be followed as shown in FIG. 20A with reduced flow rates accomplished by fine motor control. For example, the IC and EC pumps allow running from 0.005 RPM to 600 RPM all within a torque range to prevent stalling across the whole range. The flowrate range therefore is as low as 0.01 ml/min at 500 RPM on the large pumps and 0.01 mL/min at 300 RPM on the small pumps in both directions. The work performed to prevent stalling at the low speeds was due to improved stalling detection methods at slow speeds and control loop controlled differently at the slow speeds. In an example embodiment, the fluid may be pumped through the EC circulation feed loop 752, the IC circulation feed loop 753, or a combination thereof, as described with reference to FIGS. 7A-7C.

The IC and EC pumps are continuously moving at a nearly continuous speed which provides strong counterflow containment with ultra-low flowrates, as previously mentioned. If two matched pumps are duty cycling then they cannot be run against each other to hold the cells in the bioreactor without causing them to shift their position in the bioreactor. With the continuous operation of the IC and EC pumps over long periods of time, the cells are held within the bioreactor at the optimized position which conserves cell culture media overall.

Harvest synchronization, as described below, is provided by alternating pump control that can be run over long period of times. A low flowrate (approximately 1/10 the flowrate used during a step in previous strategies) achieves about 0.01 mL/min. To get those speeds an RPM of 0.005 is required at a continuous speed. Therefore, constant movement of the pump at 0.005 RPM without stalling is achieved. Please see the data found in D0000038948, D0000038355, D0000044793, D0000033725 (and all attached attachments), and D0000041290.

In an alternative embodiment, the steps of process 1900 may be followed as shown in FIG. 20A with greatly reduced time periods from the description above. For example, the IC inlet pump and the EC inlet pump may be activated at alternating time periods, where the time period is less than 10 minutes, or approximately 5 minutes. The pumps may be activated at low flow or ultra-low flow for each of the time periods. For example, law flow, or ultra-low flow may be at a flow rate of within about 0.005 RPM to 600 RPM, or within about 0.01 mL/min to 500 RPM for the standard bioreactor, or standard cell growth chamber, and within about 0.01 mL/min to 300 RPM for the small bioreactor, or small cell growth chamber. Alternating activating the IC inlet pump and the EC inlet pump for low time periods allows for intracapillary harvest synchronization. The Harvest valve and the EC outlet valve are opened with the IC inlet pump activation and the EC inlet pump activation, respectively, during the process 1900. When the IC and EC inlet pumps are running, they run at twice the commanded speed, unless twice the commanded speed exceeds the allowable range. Then the pump runs at the maximum allowable flowrate for the 5 minutes the pump is on. If stop condition is volume dependent, the Time Remaining displayed on the screen will only update when that respective pump is running.

Creating the option to cycle between the harvest outlet and the waste outlet allows the user the option to collect cell culture products of interest (such as cells, viral vectors, exosomes, conditioned media, etc.) into the harvest bag at a continuous, user-selected slow flowrate over a long period of time while still being able to feed and maintain the cell culture. This option is available in tasks for both the Standard bioreactor, or standard cell growth chamber, and Small Bioreactor, or small cell growth chamber, disposable types.

Intracapillary (IC) harvest synchronization provides continuous media over a long period of time while still feeding cells. It allows for conditioned media to feed cells which may have advantages over new media.

In an example embodiment, the fluid may be pumped through the EC circulation feed loop 752, the IC circulation feed loop 753, or a combination thereof, as described with reference to FIGS. 7A-7C.

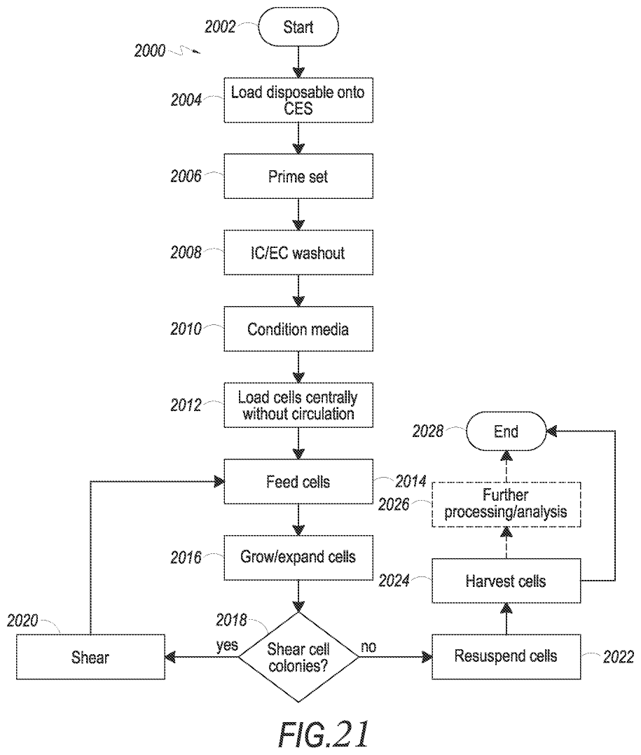

FIG. 21 illustrates example operational steps of a process 2000 for expanding cells that may be used with a cell expansion system, such as CES 500 (e.g., FIG. 5A), CES 600 (FIG. 6), or CES 700 (FIG. 7A-7C), in accordance with embodiments of the present disclosure. START operation 2002 is initiated, and process 2000 proceeds to load the disposable tubing set 2004 onto the cell expansion system. The disposable tubing set 2004 may include the cell growth chamber 100A or the cell growth chamber 100B, as illustrated in FIGS. 1C, 4B, and 4C. Next, the system may be primed 2006. In an embodiment, a user or an operator, for example, may provide an instruction to the system to prime by selecting a task for priming, for example. In an embodiment, such task for priming may be a pre-programmed task. Process 2000 then proceeds to the IC/EC Washout task 2008, in which fluid on the IC circulation loop and on the EC circulation loop is replaced. The replacement volume is determined by the number of IC Volumes and EC Volumes exchanged.

Next, to maintain the proper or desired gas concentration across the fibers in the bioreactor membrane, the condition media task 2010 may be executed to allow the media to reach equilibrium with the provided gas supply before cells are loaded into the bioreactor. For example, rapid contact between the media and the gas supply provided by the gas transfer module or oxygenator is provided by using a high EC circulation rate. The system may then be maintained in a proper or desired state until a user or operator, for example, is ready to load cells into the bioreactor. In an embodiment, the system may be conditioned with complete media, for example. Complete media may be any media source used for cell growth. In an embodiment, complete media may comprise alpha-MEM (α-MEM) and fetal bovine serum (FBS), for example. Any type of media understood by those of skill in the art may be used.

Process 2000 next proceeds to loading cells centrally without circulation 2012 into the bioreactor from a cell inlet bag, for example. In embodiments, a “load cells centrally without circulation” task may be used, in which a first volume of fluid at a first flow rate comprising a plurality of cells may be loaded into the cell expansion system, in which the cell expansion system comprises a cell growth chamber. A second volume of fluid at a second flow rate comprising media may then be loaded into a portion of a first fluid circulation path, for example, to position the first volume of fluid in a first portion of the cell growth chamber. In an embodiment, the first portion of the cell growth chamber or bioreactor may comprise about a central region of the bioreactor. In an embodiment, the first volume is the same as the second volume. In an embodiment, the first flow rate is the same as the second flow rate. In another embodiment, the first volume is different from the second volume. In another embodiment, the first flow rate is different from the second flow rate. In an embodiment, the sum of the first volume and the second volume may equal a percentage or proportion of the volume, e.g., total volume, of the first fluid circulation path, for example. For example, the sum of the first volume and the second volume may be about 50% of the volume, e.g., total volume, of the first fluid circulation path, for example. In an embodiment, fluid in the first fluid circulation path flows through an intracapillary (IC) space of a bioreactor or cell growth chamber. In an embodiment, fluid in a second fluid circulation path flows through an extracapillary (EC) space, for example, of a cell growth chamber or bioreactor. In an example embodiment, the fluid may be fed 916 through the EC circulation feed loop 752, the IC circulation feed loop 753, or a combination thereof, as described with reference to FIGS. 7A-7C. In an embodiment, the sum of the first volume and the second volume may be about 50%, or another percentage or proportion according to embodiments, of the volume of the intracapillary (IC) loop, for example. In an embodiment, the sum of the first volume and the second volume may be about 50%, or another percentage or proportion according to embodiments, of the volume of another fluid path, loop, etc., as applicable. Other percentages or proportions may be used, including, for example, any percentage between and including about 1% and about 100%, in accordance with embodiments.

Following the loading of the cells 2012, process 2000 next proceeds to feed the cells 2014. The cells may be grown/expanded 2016. While step 2016 is shown after step 2014, step 2016 may occur before, or simultaneous with, step 2014, according to embodiments. Next, process 2000 proceeds to query 2018 to determine whether any cell colonies, micro-colonies, or clusters have formed. A cell colony, micro-colony, or cluster may be a group of one or more attached cells. If a cell colony, micro-colony, or cluster has formed, process 2000 proceeds “yes” to shear 2020 any cell colonies, micro-colonies, or clusters. For example, after expanding a plurality of cells for a first time period, the cells may be circulated at a first circulation rate during a second time period to reduce a number of cells in a cell colony, micro-colony, or cluster. In embodiments, the circulating the cells at the first circulation rate may cause the cell colony to incur a shear stress, in which one or more cells in the cell colony may break apart from the cell colony. In an embodiment, reducing the number of cells in the cell colony, micro-colony, or cluster may provide a single cell suspension, for example. In embodiments, circulating the cells to shear any colony, micro-colony, or cluster 2020 may be used every two (2) days, for example, during cell culture to maintain uniform cell density and nutrient diffusion. Other time periods may also be used according to embodiments. In an embodiment, such shearing of any micro-colonies, colonies, or clusters may begin on or after Day 4, for example. Other days or time periods on which to begin such shearing may be used according to embodiments. Following shearing 2020, process 2000 may next return to feed cells 2014.

If it is determined at query 2018 not to shear any cell colonies or clusters, or if none exist, for example, process 2000 proceeds “no” to resuspend cells 2022. In embodiments, circulating the cells may be used to uniformly resuspend those cells that may be loosely adhered during culture. In embodiments, step 2022 may include circulating the cells to uniformly resuspend those cells that may be loosely adhered prior to initiating a harvest task, or other task to remove cells from the bioreactor. Following the resuspension of the cells 2022, process 2000 next proceeds to harvest the cells 2024. Further processing of the removed cells or other analysis may optionally be performed at step 2026, and process 2000 may then terminate at END operation 2028. If it is not desired to perform further processing/analysis, process 2000 terminates at END operation 2030.

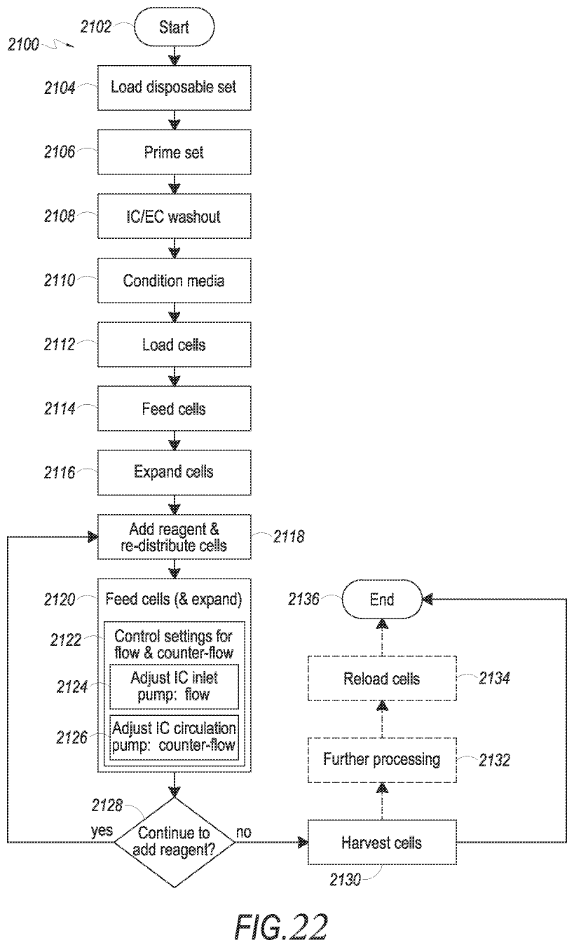

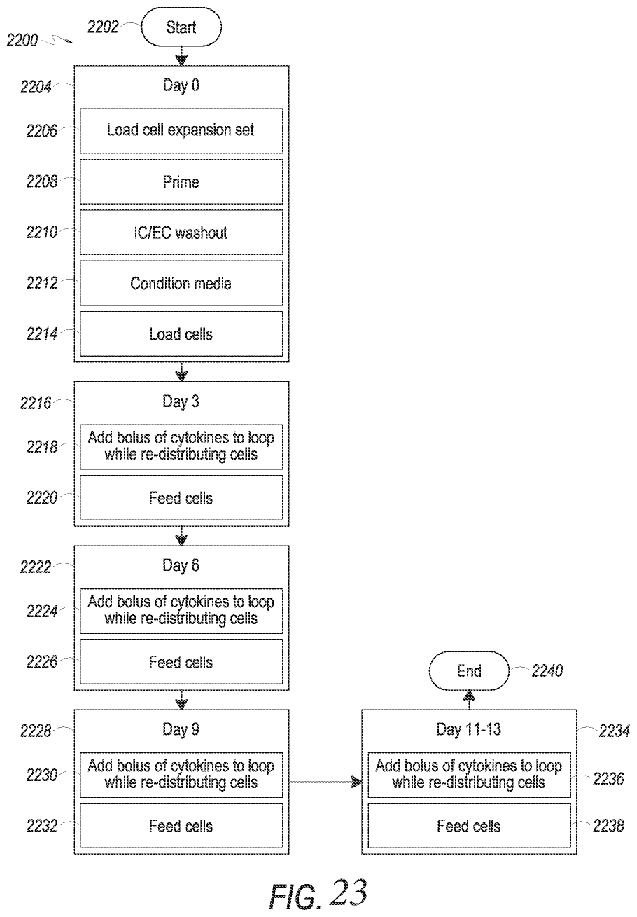

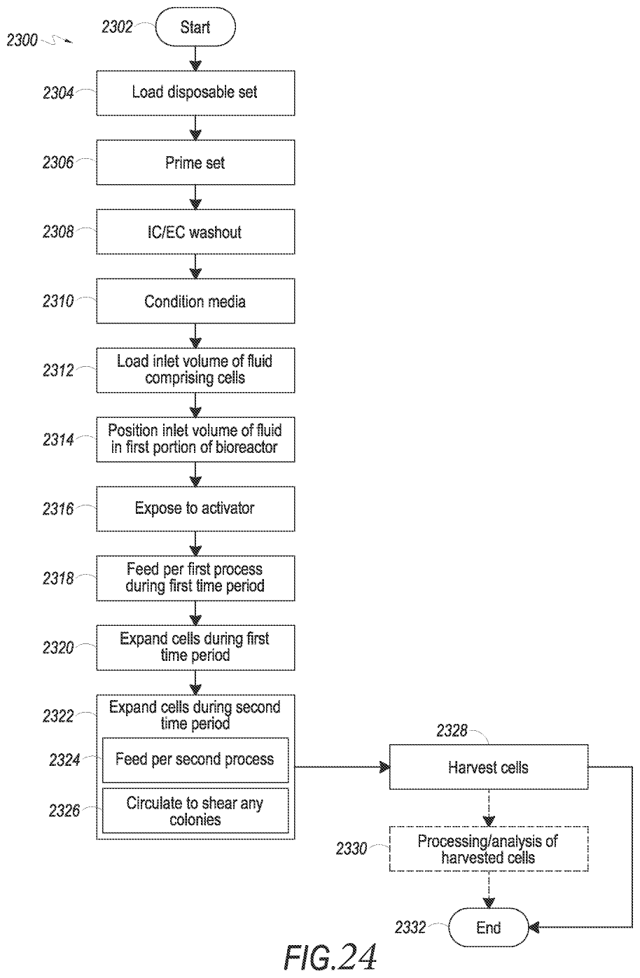

Turning to FIG. 22 and process 2100, START operation is initiated 2102, and process 2100 proceeds to load a disposable set at 2104 onto a cell expansion system, according to embodiments. The disposable set may include the cell growth chamber 100A or the cell growth chamber 100B, as illustrated in FIGS. 1C, 4B, and 4C. The disposable set may then be primed 2106, and an IC/EC washout step 2108 may occur. The media may next be conditioned 2110. Next process 2100 proceeds to load cells 2112, e.g., suspension or non-adherent cells, such as T cells or Tregs. In an embodiment, such cells may be loaded 2112 by a “load cells centrally without circulation” task. In another embodiment, such cells may be loaded 2112 by a “load cells with uniform suspension” task.

Next, process 2100 proceeds with beginning to feed the cells 2114, which may begin on Day 0, according to embodiments. In an example embodiment, the fluid may be fed 916 through the EC circulation feed loop 752, the IC circulation feed loop 753, or a combination thereof, as described with reference to FIGS. 7A-7C. The cells may grow and expand 2116, and by Day 3, for example, it may be desired to add a bolus of a fluid to the IC loop and re-distribute the cells 2118. In an embodiment, such bolus of fluid may comprise a reagent, such as cytokines or other growth factor(s). In another embodiment, such bolus of fluid may comprise a reagent and base media, for example.

Following such bolus addition and re-distribution of cells, process 2100 next proceeds to feeding 2120 the cells again, in which such feeding may occur on Day 3, for example. With such feeding 2120, the parameters of the system, such as one or more pumps controlling flow rate, may be controlled 2122 to achieve complementary flow and counter-flow settings for fluid moving into the bioreactor from both the IC inlet port and the IC outlet port of the bioreactor. For example, the IC inlet pump 2124 may be adjusted or directed to produce a flow, and the IC circulation pump may be adjusted or directed 2126 to produce a counter-flow. For example, an IC inlet pump rate of 0.1 mL/min may be matched, or closely or substantially matched, to a complementary IC circulation pump rate of −0.1 mL/min to maintain cells in the bioreactor during the growth phase of the cell culture, which may be Days 4-7, for example, in embodiments. Alternatively, in an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture. Such control of settings 2122 may allow for counteracting any forces associated with a loss of cells from the IC outlet port of the bioreactor.

Process 2100 next proceeds to query 2128, in which it is determined whether to continue to add reagent or other bolus addition on other days or other time intervals, for example. If it is desired to add additional reagent or other bolus and re-distribute cells, process 2100 branches “yes” to add reagent and re-distribute cells 2118. For example, such bolus addition and re-distribution of cells may next occur on Days 6 and 9, according to embodiments.

If, or once, it is not desired to continue adding a bolus, e.g., reagent, and re-distributing the cells, process 2100 proceeds “no” to harvest the cells 2130, in which the cells may be transferred to a harvest bag(s) or container(s). Process 2100 then terminates at END operation 2136.

Alternatively, from harvest operation 2130, process 2100 may optionally proceed to allow for further processing/analysis 2132. Such further processing 2132 may include characterization of the phenotype(s), for example, of the harvested cells, e.g., T cells or Tregs. From optional further processing/analysis step 2132, process 2100 may proceed to optionally reload any remaining cells 2134. Process 2100 may then terminate at END operation 2136.

Process 2200 illustrates operational steps for a process of expanding cells in cell expansion system according to embodiments of the present disclosure. Process 2200 may be used in some embodiments to expand T cells. As illustrated, various steps may be performed over the course of a 14-day protocol to expand the cells. START operation 2202 is initiated and process 2200 proceeds to Day 0, where a disposable set is loaded onto a cell expansion system 2206. The disposable set may include the cell growth chamber 100A or the cell growth chamber 100B, as illustrated in FIGS. 1C, 4B, and 4C. The disposable set may then be primed 2208, in which the set may be primed 2208 with PBS (e.g., Lonza Ca2+/Mg2+-free), for example. In preparation for the loading of cells, the priming fluid may be exchanged using an IC/EC washout 2210. For example, the PBS in the system may be exchanged for TexMACS GMP Base Medium, according to one embodiment. The media may next be conditioned 2212. The condition media 2212 may be performed to allow the media to reach equilibrium with provided gas supply before cells are loaded into a bioreactor.

Next on Day 0, process 2200 proceeds to load cells 2214, e.g., suspension or non-adherent cells, such as T cells or Tregs. In an embodiment, such cells may be loaded 2214 by a “load cells centrally without circulation” task. In another embodiment, such cells may be loaded 2214 by a “load cells with uniform suspension” task.

At Day 3 2216, a bolus of cytokines may be added while the cells are redistributed 2218. In embodiments, the redistribution of cells may be performed in combination with the bolus addition to mix the cells and more thoroughly expose the cells to the cytokines (e.g., IL-2) that may be in the bolus addition. In embodiments, the redistribution may also break up colonies or clusters of cells that may have formed. In embodiments, the redistribution may occur first by circulating the cells in a fluid circulation path. The bolus addition may then be added in the process of pushing the cells back into the bioreactor, such as by introducing fluid into a fluid circulation path to push cells back into the bioreactor. After the redistribution and bolus addition 2218, process 2000 proceeds to feed cells 2220.

The cells may again be redistributed 2224 with another bolus addition at Day 6 2222. The redistribution may break up colonies or clusters of cells that may have formed during Days 3-5. The bolus addition may expose the cells to additional reagents that promote expansion. Process 2000 proceeds to feed cells 2226 at Day 6. At Day 9 2228, the cells may once again be redistributed 2230 with a bolus addition. The redistribution may break up colonies or clusters of cells that may have formed during Days 6-8. The bolus addition may expose the cells to additional supplements that promotes expansion. Process 2000 proceeds to feed cells 2232 at Day 9.

At Day 11-13 2234, the cells may again be redistributed 2236 with a bolus addition. The redistribution may break up colonies or clusters of cells that may have formed during Days 9-10. The bolus addition may expose the cells to additional reagents that promote expansion. Process 2000 then proceeds to feed cells 2238. In embodiments, the steps 2236 and 2238 may be performed on each of Day 11, Day 12, and Day 13. This may occur as a result of the cells expanding during Days 0-10 and there being larger numbers of cells in the bioreactor. Performing the redistribution and bolus addition of cells may promote expansion of the cells by breaking up colonies and clusters of cells more often and mixing them with reagents in the bolus addition to promote cell expansion. Process 2200 terminates at END operation 2240.

Process 2300 illustrates operational steps for a process of expanding cells, e.g., suspension or non-adherent cells, in a cell expansion system according to embodiments of the present disclosure. Process 2300 may be used in some embodiments to expand T cells, such as Tregs. The combination of steps of process 2300 may allow the expansion of the cells to useful clinical amounts using initial low seeding densities.

START operation 2302 is initiated and process 2300 proceeds to load disposable set 2304 onto a cell expansion system. The disposable set may include the cell growth chamber 100A or the cell growth chamber 100B, as illustrated in FIGS. 1C, 4B, and 4C. The disposable set may then be primed 2206, in which the set may be primed 2206 with PBS (e.g., Lonza Ca2+/Mg2+-free), for example. In preparation for the loading of cells, the priming fluid may be exchanged using an IC/EC washout 2308. For example, the PBS in the system may be exchanged for TexMACS GMP Base Medium, according to one embodiment. The media may next be conditioned 2310. The condition media 2310 may be performed to allow the media to reach equilibrium with a provided gas supply before cells are loaded into a bioreactor.

Process 2300 proceeds to 2312 load inlet volume of fluid with cells. In embodiments, the cells may comprise non-adherent cells, such as one or more types of T cells, e.g., Tregs. In one embodiment, the cells comprise Tregs. Embodiments may provide for the inlet volume of fluid with the cells to be loaded through an IC inlet path utilizing an IC inlet pump and into an IC circulation path. In embodiments, the load inlet volume 2312 is loaded without activating an IC circulation pump.

Process 2300 proceeds to positioning the inlet volume in a first portion of a bioreactor 2314. In embodiments, the positioning may be performed by introducing a second volume of fluid, which may comprise media and may be introduced into a portion of the IC circulation path to push the inlet volume with the cells into the first position in the bioreactor. In embodiments, the inlet volume of fluid and the second volume of fluid may be the same. In other embodiments, the inlet volume of fluid and the second volume of fluid may be different. In yet other embodiments, a sum of the inlet volume of fluid and the second volume of fluid may be equal to a percentage of a volume of the IC circulation path.

Following the position inlet volume 2314, process 2300 proceeds to expose cells to activator 2316 in order to activate the cells to expand. The cells may be exposed to an activator 2316 that is soluble in some embodiments. The activator, which may include antibody complexes in some embodiments, may be added to the media and may be included in the inlet volume or added later, such as with the second volume. In embodiments, the activator may be a human antibody CD3/CD28/CD2 cell activator complex, for example.

Process 2300 proceeds to feed the cells per a first process during a first time period 2318. In an example embodiment, the cells may be fed 916 through the EC circulation feed loop 752, the IC circulation feed loop 753, or a combination thereof, as described with reference to FIGS. 7A-7C. In an embodiment, the cells may be fed at a minimum or low feed rate, for example, where the cell population is beginning to grow/expand for a first time period 2320, and a minimum or low feed rate is able to meet the demands of such population. For example, an IC inlet pump rate of +0.1 mL/min may be used during such first time period. If it is desired to reduce the loss of cells from the hollow fiber membrane bioreactor during such first time period, the IC inlet pump rate of +0.1 mL/min may be matched, or closely or substantially matched, to a complementary IC circulation pump rate of −0.1 mL/min to maintain cells in the bioreactor during the growth phase of the cell culture. Alternatively, in an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture.

From 2320, process 2300 proceeds to expand cells during a second time period 2322. Expanding during the second period of time 2322 may also involve feeding the cells per a second process during the second time period 2324. In an embodiment, such second process may involve feeding the cells at substantially the same feed rates as during the first time period, for example. In another embodiment, the second process may involve feeding the cells at different feed rates as compared to the feed rates used during the first time period. For example, the feed rates may increase as a result of the expansion of the cells during the first time period.

While expanding the cells during the second time period 2322, the cells may also be circulated to shear cells colonies or cell clusters 2326. Step 2326 may involve circulating the cells in the IC circulation path to shear any colonies or clusters that may have formed during the first time period. The shear colonies or clusters 2326 step may reduce a number of cells in a cell colony or cell cluster. In embodiments, the circulate to shear 2326 may cause cell colonies to incur a shear stress, causing one or more cells in the cell colony to break apart from the cell colony.

Process 2300 may next proceed to harvest operation 2328, in which the cells may be transferred to a harvest bag(s) or container(s). In embodiments, a therapeutic dose of cells may be harvested. In embodiments, the cells harvested at operation 2328 may be on the order of 1×10⁹ cells. The harvested cells may have viabilities between about 75% and about 95%, in embodiments.

Process 2300 may then optionally proceed to allow for further processing/analysis 2330. Such further processing may include characterization of the phenotype(s), for example, of the harvested cells, e.g., T cells or Tregs. In one embodiment, the harvested cells may express biomarkers consistent with Tregs. For example, the cells may express CD4+, CD25+, and/or FoxP3+ biomarkers. In embodiments, the harvested cells may include the CD4+CD25+ phenotype at a frequency of above about 80%. In other embodiments, the cells may include the CD4+FoxP3+ phenotype at a frequency of above about 55%. Process 2300 may then terminate at END operation 2332.

The operational steps depicted in the above figures are offered for purposes of illustration and may be rearranged, combined into other steps, used in parallel with other steps, etc., according to embodiments of the present disclosure. Fewer or additional steps may be used in embodiments without departing from the spirit and scope of the present disclosure. Also, steps (and any sub-steps), such as priming, conditioning media, loading cells, for example, may be performed automatically in some embodiments, such as by a processor executing pre-programmed tasks stored in memory, in which such steps are provided merely for illustrative purposes. Further, the example pump rate settings for feeding cells depicted in FIG. 11B, for example, are offered for purposes of illustration. Other pump rates, flow rates, directions, etc. may be used in accordance with embodiments of the present disclosure.

Examples and further description of tasks and protocols, including custom tasks and pre-programmed tasks, for use with a cell expansion system are provided in U.S. patent application Ser. No. 13/269,323 (“Configurable Methods and Systems of Growing and Harvesting Cells in a Hollow Fiber Bioreactor System,” filed Oct. 7, 2011) and U.S. patent application Ser. No. 13/269,351 (“Customizable Methods and Systems of Growing and Harvesting Cells in a Hollow Fiber Bioreactor System,” filed Oct. 7, 2011), which are hereby incorporated by reference herein in their entireties for all that they teach and for all purposes.

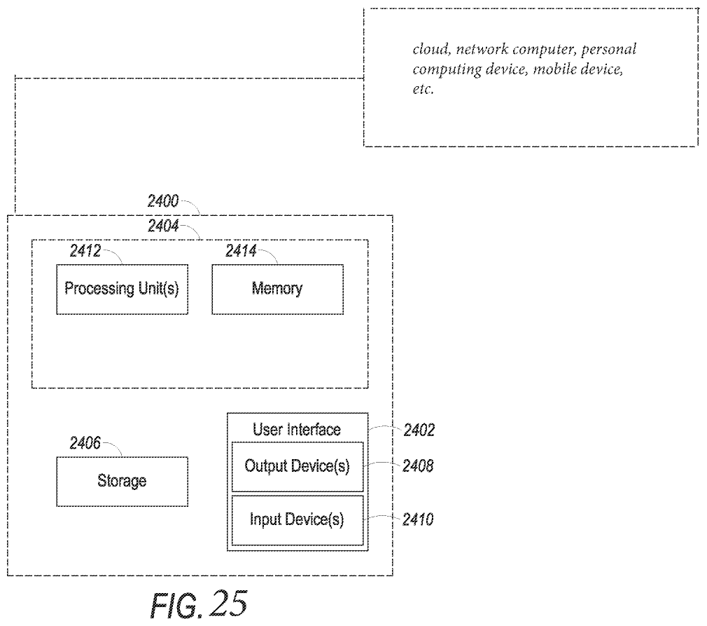

Next, FIG. 25 illustrates example components of a computing system 2400 upon which embodiments of the present disclosure may be implemented. Computing system 2400 may be used in embodiments, for example, where a cell expansion system uses a processor to execute tasks, such as custom tasks or pre-programmed tasks performed as part of processes such as processes illustrated and/or described herein. In embodiments, pre-programmed tasks may include, follow “IC/EC Washout” and/or “Feed Cells,” for example.

The computing system 2400 may include a user interface 2402, a processing system 2404, and/or storage 2406. The user interface 2402 may include output device(s) 2408, and/or input device(s) 2410 as understood by a person of skill in the art. Output device(s) 2408 may include one or more touch screens, in which the touch screen may comprise a display area for providing one or more application windows. The touch screen may also be an input device 2410 that may receive and/or capture physical touch events from a user or operator, for example. The touch screen may comprise a liquid crystal display (LCD) having a capacitance structure that allows the processing system 2404 to deduce the location(s) of touch event(s), as understood by those of skill in the art. The processing system 2404 may then map the location of touch events to UI elements rendered in predetermined locations of an application window. The touch screen may also receive touch events through one or more other electronic structures, according to embodiments. Other output devices 2408 may include a printer, speaker, etc. Other input devices 2410 may include a keyboard, other touch input devices, mouse, voice input device, etc., as understood by a person of skill in the art. For example, the user interface 2402 may be the user interface 264 described with reference to FIG. 2A. The user interface 2402 may include different screens for different portions of a task/protocol/process/method, for different user inputs or requests, etc.

Processing system 2404 may include a processing unit 2412 and/or a memory 2414, according to embodiments of the present disclosure. The processing unit 2412 may be a general purpose processor operable to execute instructions stored in memory 2414. Processing unit 2412 may include a single processor or multiple processors, according to embodiments. Further, in embodiments, each processor may be a multi-core processor having one or more cores to read and execute separate instructions. The processors may include general purpose processors, application specific integrated circuits (ASICs), field programmable gate arrays (FPGAs), other integrated circuits, etc., as understood by a person of skill in the art.

The memory 2414 may include any short-term or long-term storage for data and/or processor executable instructions, according to embodiments. The memory 2414 may include, for example, Random Access Memory (RAM), Read-Only Memory (ROM), or Electrically Erasable Programmable Read-Only Memory (EEPROM), as understood by a person of skill in the art. Other storage media may include, for example, CD-ROM, tape, digital versatile disks (DVD) or other optical storage, tape, magnetic disk storage, magnetic tape, other magnetic storage devices, etc., as understood by a person of skill in the art.

Storage 2406 may be any long-term data storage device or component. Storage 2406 may include one or more of the systems described in conjunction with the memory 2414, according to embodiments. The storage 2406 may be permanent or removable. In embodiments, storage 2406 stores data generated or provided by the processing system 2404.

Computing system 2400 may be in communication with a cloud, a network computer, a personal computing device, a mobile device, etc., though a wireless network, a Bluetooth network, or other network system. Alternatively, computing system 2400 may be in communication with a personal computing device, a network computing device, a mobile device, etc., through a hard-wired connection.

Processing system 2404 may control pump activation, speed, and fluid flow. Processing system 2404 may control pump activation/speed to provide ultra-low feed rates with continuous motion (instead of stepped motion) with the control loop of the pump process. Continuous, consistent movement (compared to period/stepped movement) facilitates finer pump control and lower feed rates. Operation may be switched between two pumps operating at a low flow rate to facilitate continuous harvesting of cells. In an example embodiment, the first IC inlet pump may cause a volumetric flow rate of less than 0.1 mL/min, or about 0.01 mL/min to enter the IC inlet port (701A) with the IC circulation pump causing a complementary IC circulation volumetric flow rate or fluid flow rate of less than −0.1 mL/min, or about −0.01 mL/min, to enter the IC outlet port (701B), in which the negative symbol (“−”) used in −0.01 mL/min, for example, indicates a direction of the IC circulation pump to cause or produce a counter-flow rate to maintain cells in the bioreactor during the growth phase of the cell culture.

Processing system 2404 may control pump activation/speed for counterflow containment. Counterflow containment with ultra-low flow allows concentration of cells in a desired area. Counterflow containment conserves cell culture media and allows for lower protein concentrations in the system. The ability to provide continuous motion allows for the lower protein concentrations. Opposite pumps pump in opposite directions to cluster cells.

Processing system 2404 may be configured to run various tasks/methods/processes/protocols that are input by the user or stored as preset tasks/methods/processes/protocols in the memory 2414. For example, processing system 2404 may be configured to run or coordinate execution of a cell processing application (CPA). The cell processing application enables tracking and logging of the user's materials, procedures, user logins, and more. The system can be used to push protocols to a fleet of machines that are all identical and therefore saving a lot of time setting up a validated protocol to run on a fleet of machines. All reports can then be pulled back to the application and a live readout of the fleet can be seen remotely on the application. Temperatures and pressures can be updated at a configurable rate to the application, but also alarms, and warnings are sent to the application. For example, the application may push the alarm as a remote alarm to one or more users for recording or remediation. For example, the remote alarm may be an email, a text message, or other digital alert sent to the user. CPA also connects FINIA® and Quantum Flex® to hold all of the user's reports in a unified application.

CPA has been tested in the following protocols and reports: User Access Control Protocol: D0000028598, User Access Control Report: D0000043953, Remote Alarming Protocol: D0000028623, Remote Alarming Report: D0000043950, Barcode Protocol: D0000028807, Barcode Report: D0000044530, Protocol Task Management Protocol: D0000028798, Protocol Task Management Report: D0000044037, Device Settings Protocol: D0000028621, Device Settings Report: D0000045376, EOR Protocol: D0000028620, EOR Report: D0000045378, D0000047097: Network Performance, D0000047098: Multiple Devices, D0000047099: Access and Audit, D0000047100: Data and Reports.

The cell processing application may include features of tracking and data management. Tracking and logging may be enabled through CPA. Tracking and logging may be done periodically (e.g., scheduled) or on demand A user may schedule times to record based on trigger events or periodic times.

The cell processing application may also perform fleet control. CPA unifies modular cell therapies including a CES, e.g., Quantum Flex® Cell Expansion System, and FINIA®, to name a few. Thus, the CES described herein, for example CES 100, 700, etc., is a single module in a modular system controlled by the cell processing application. For example, CPA may control a fleet of up to 100 or more devices. Using CPA, the same instructions may be sent to multiple machines rather than individual machines to streamline and ensure consistency in operation.

The cell processing application may include custom tasks/methods/processes/protocols. Users may draft custom protocols or custom tasks. Tasks may be detailed steps for a user-defined process. Protocols may be a compilation of tasks needed to complete a test or cell expansion process, etc., saved in a single file. The memory 2414 may store predefined, prewritten, or stock protocols and tasks. Stock tasks may be compiled by a user into custom protocols. Protocols in the CPA system may be modified in real time and re-uploaded to the fleet of devices. Tasks and protocols may be written, modified, selected both on the user interface or on an external computer or handheld device.

The cell processing application may include different user profiles, accounts, and access. Each user is assigned one of a selection of predefined roles or a custom role. Each role includes a set of permissions which may be customizable by an administrator. The permission may include control levels (e.g., read-only, write, etc.) of access. One user may have multiple roles. The CPA may require user authentication for security measures.

The cell processing application may trigger and send remote notifications and alarms. An email or other notification (digital notification, text, etc.) may be sent to the user so the condition can be monitored/inspected/etc. without the user having direct access or contact with the machine. The user may be able to communicate remotely with the machine, including reviewing real-time data through the cell processing application. The user may be able to remotely send commands to the machine, such as ignore alarm, stop testing, modify the protocol or test, etc. This is more efficient for the user.

The cell processing application controls network access for software updates. New software may be pushed from the cloud, a remote computer, or remote device to the CES or other devices.

The cell processing application may receive data from a barcode scanner. The barcode scanner may be one of the input device(s) 2410 previously described. The disposable packages, media, cells, etc., each include a barcode with product information. The CPA may check the data from the barcode scanner to ensure the correct component or media is being used. Barcodes may be stored in the memory 2414 or the CPA for future reference, such as product recalls, or expansion data. The CPA may adjust task/method/process/protocol configuration based on data from the barcode scanner.

Computing system 2400 may increase process efficiency by driving contact between “reagents of interest” (virus particles, transfection reagent, secondary cell type, differentiation reagent, induction reagent, etc.) and adherent or suspension cells in a hollow fiber bioreactor (HFB). Cells (adherent or suspension) are seeded in the intracapillary (IC) side of the HFB. The reagent of interest is introduced to the IC side of the HFB. Countercurrent flow (split by driving the IC Inlet pump and the IC circulation pump in opposite directions—Inlet=positive flow, Circ=negative flow) is used to drive active contact between the reagent of interest and the population of cells. This process can continue as long as necessary.

Many processes in cell culture require the exposure of the cell population to a particular suspended element to modify the cells or produce a secondary product. For example, in order to produce a viral vector product, the population of cells must be exposed to active viral particles that will replicate inside the cells; to transfect a cell by introducing a novel gene to the cell, the cells must be exposed to both the genes of interest (GOI) and the reagents required to bring the GOI inside the cell for translation. Both of these example processes have variable efficiencies based upon the environment in which the process occurs. Many of these passive models for these types of processes rely on a high degree of random chance to drive interactions between the reagent of interest (ROI) and the cell population. These passive models result in lower efficiencies of viral integration/plasmid transfection/GOI expression.

The process described herein is intended to increase the efficiency of these processes by driving active contact between the cell population and the ROI. Cells (adherent or suspension) are seeded in the fibers of the HFB. Once cells are established, ‘reagent of interest’ (virus particles, transfection reagent, secondary cell type, differentiation reagent, induction reagent, etc.) is introduced to the HFB. Vehicle fluid is drawn out of the 2-port bag. Flow is split by the Inlet and Circulation pumps. Vehicle fluid enters the IC side of the HFB from both sides. The IC Waste Valve is closed, so fluid must leave the HFB through pores in the HFB membrane going from IC □EC sides. Vehicle fluid is recirculated from the EC side back to 2-port bag. ‘reagent of interest’ is contained in the IC loop due to the molecules being larger than the membrane pores and is forced against the layer of cells on the membrane wall driving contact between the ‘reagent of interest’ and the cell population. Continue for as long as required.

### EXAMPLES

The following description includes some examples of protocols/methods/processes that may be used with a cell expansion system, such as CES 500 (e.g., FIGS. 5A, 5B, 5C) and/or CES 600 (FIG. 6), for example, which implements aspects of the embodiments. Although specific features may be described in the examples, such examples are provided merely for illustrative and descriptive purposes. For example, while examples may provide for the expansion of T cells and/or Treg cells, other and/or additional cell types and/or combinations thereof may be used in other embodiments. Although specific parameters, features, and/or values are described, e.g., use of a CES, e.g., Quantum® Cell Expansion System, according to some embodiments, these parameters, features, and/or values, etc., are provided merely for illustrative purposes. The present disclosure is not limited to the examples and/or specific details provided herein.

Further, the examples provided herein are not intended to limit other embodiments, which may include different or additional steps, parameters, or other features. The example methods or protocols, including the steps (and any sub-steps), may be performed automatically in some embodiments, such as by a processor executing pre-programmed tasks stored in memory. In other embodiments, the steps (and any sub-steps) may be performed through the combination of automated and manual execution of operations. In further embodiments, the steps (and any sub-steps) may be performed by an operator(s) or user(s) or through other manual means.

While example data may be provided in such examples, such example data are provided for illustrative purposes and are not intended to limit other embodiments, which may include different steps, parameters, values, materials, or other features.

In some examples, the protocol package(s) or method(s) for the smaller bioreactor, or smaller cell growth chamber, may be the same tasks as for the standard bioreactor, or standard cell growth chamber. In some protocol package(s) or method(s) for the smaller bioreactor, or smaller cell growth chamber, the following differences may be included: 1. reduced flowrates when draining the Air Removal Chamber (ARC) in ARC Management steps and when flow is forced across the membranes. This is because the small bioreactor has a smaller fiber surface area, so the flowrates are reduced to avoid high pressures. 2. Different default selections based on the differences in the disposable sets (i.e., where applicable, flowrates and volume stop conditions are scaled down per differences in volumes of certain portions of the set). 3. Ranges are opened to allow users the option to scale down flows up to 1/10th of the Standard Bioreactor tasks.

In some example embodiments, protocol packages(s) or method(s) may include: 1. Default (Template) Adherent Cell Expansion Protocol; 2. Default (Template) Suspension Cell Expansion Protocol; 3. Default (Template) Custom One-Step Protocol; 4. Custom Step 1-10 Tasks with increased options available compared to Legacy Quantum custom task; 5. Load Cells with Multiple Distribution Cycles (task created by scientific team to optimize seeding for MSCs); 6. Load and Position Cells (task created by scientific team to optimize loading for T-Cells); 7. Feed Non-Adherent Cells (task created by scientific team to optimize feeding for T-Cells); and/or 8. Circulate and Position Cells (task created by scientific team to optimize sampling during T-Cell expansion)

### Example 1

Methods

General Treg Cell Culture

Immunomagnetic-isolated CD4⁺CD25⁺ Tregs may be acquired from healthy adult donor peripheral blood by leukapheresis (HemaCare Corporation, Van Nuys, CA) and may be subsequently expanded at Terumo BCT at a concentration of 1.0×10⁵ cells/mL in sterile-filtered TexMACS™ GMP Medium supplemented using three T25 flasks (7 mL/flask) with the recombinant human IL-2 IS Premium grade cytokine at 200 IU/mL (Miltenyi Biotec GmbH, Bergisch Gladbach) and Gibco PSN 100× antibiotic mixture (ThermoFisher Scientific, Waltham, MA). The actively growing Treg cell suspension may be subsequently used as the inoculum in each of the three (3) Quantum Cell Expansion System experimental runs. Tregs for both the inoculum and the Quantum System expansion may be co-stimulated using a soluble tetrameric Immunocult™ human antibody CD3/CD28/CD2 cell activator complex (Stem Cell Technologies, Vancouver, BC) at 25 μL/mL in the absence of microbeads. Co-stimulation may be performed on Days 0 and 9 for the Treg inoculum and on Day 0 for the Quantum System Treg expansion. The Quantum System HFM bioreactor may be characterized by an intracapillary loop volume of 177.1 mL and surface area of 21,000 cm².

Quantum System Treg Expansion

According to embodiments, two (2 L) bags of sterile filtered media may be prepared for the Treg scale up expansion in the Quantum System using the Quantum Media Bag 4 L Set (Cat. 21021). One 2 L bag of complete media containing TexMACS GMP, IL-2 and PSN antibiotics may be used to supply the IC compartment and one 2 L of base media containing TexMACS GMP and PSN antibiotics may be used to supply the EC inlet compartment of the bioreactor. After priming the Quantum System with PBS (Lonza Cat. 17-516Q, Walkersville, MD), media bags may be connected to the appropriate IC and EC inlet lines using the TSCD-Q Terumo Sterile Welder. The complete media may be protected from exposure to light.

The total cell load for each run (4.5-6.5×10⁷ Tregs) may be resuspended, using aseptic technique, in 50 mL of complete medium with a Quantum Cell Inlet Bag (Cat. 21020) for introduction into the Quantum System bioreactor. Additional disposable bags, such as the Quantum CES Media Bag 4 L (Cat. 21021) and Waste Bag 4 L (Cat. 21023) may also be used during the Treg scale-up expansion runs.

At the completion of the “Load Cells Centrally without Circulation” Task, the Quantum System runs (n=3) may be seeded with Tregs at a concentration of 2.5-3.7×10⁵ cells/mL in 177 mL of complete medium or an average of 2.1-3.1×10³ cells/cm² within the lumen or the intracapillary (IC) compartment of the hollow fiber membrane bioreactor.

Day 0-4:

Example Quantum Custom Task

Feed Cells, Modified.

IC/EC Exchange & Condition Media for Regulatory T cells, Example.

The TexMACS GMP Complete Medium with IL-2 supplement (200 IU/mL) Media bag may be attached to the IC Media line of the Quantum System with the Terumo BCT TSCD-Q sterile welder. The TexMACS Base Media may be attached to the EC Media line. The IC/EC Washout and Condition Media Tasks may be performed, respectively. Complete Media may be used for IC Exchange or Washout and Base Media may be used for the EC Exchange or Washout to conserve the amount of IL-2 and activator complex.

The system may be placed on modified “Feed Cells” prior to introducing cells. The IC inlet rate (Q₁) and IC circulation rate (Q₂) may be increased in a matched rate to 0.2, 0.3, and 0.4 mL/min on Days 5, 6, and 7, but opposite direction on Days 4, 5, 6 or as needed to keep the lactate level between 5-8 mmol/L.

TABLE 1
Feed Cells, Modified, Example.
Table 1: Feed Cells

| Setting | Step 1 |
|---|---|
| IC Inlet | IC Media |
| IC Inlet Rate | 0.1 |
| IC Circulation Rate | −0.1 |
| EC Inlet | None |
| EC Inlet Rate | 0.00 |
| EC Circulation Rate | 100 |
| Outlet | EC Waste |
| Rocker Control | No Motion: |
| | (0°) |
| Stop Condition | Manual |
| Estimated Fluid | Unknown |
| Omit or Include | Include |

Example Quantum Custom Task:

Load Cells Centrally without Circulation, Example.

Purpose: This task may enable suspension cells to be centrally distributed within the bioreactor membrane while allowing flow on the extracapillary (EC) circulation loop. The pump flow rate to the IC loop may be set to zero.

Prior to loading the cells into the Quantum systems using the Load Cells without Circulation, the modifications to the task may be entered.

TABLE 2
Load Cells Centrally without Circulation, Modifications, Example
Table 2: Solutions for Loading Suspension Cells

| | | Volume |
|---|---|---|
| | | (estimate based on factory |
| Bag | Solution in Bag | default values) |
| Cell Inlet | 50 mL | N/A |
| Reagent | None | N/A |
| IC Media | Serum-Free Media | 6 mL/hour |
| Wash | None | N/A |
| EC Media | None | N/A |

TABLE 3
Load Cells Centrally without Circulation, Example
Table 3: Custom Task 8 Load Cells without Circulation

| Setting | Step 1 | Step 2 |
|---|---|---|
| IC Inlet | Cell | IC Media |
| IC Inlet Rate | 50 | 50 |
| IC Circulation Rate | 0 | 0 |
| EC Inlet | None | None |
| EC Inlet Rate | 0.00 | 0.00 |
| EC Circulation Rate | 30 | 30 |
| Outlet | IC Waste | IC Waste |
| Rocker Control | In Motion: | In Motion: |
| | (−90 to 180°) | (−90 to 180°) |
| | Dwell Time | Dwell Time |
| | 1 sec | 1 sec |
| Stop Condition | Empty Bag | IC Volume: |
| | | 57.1 mL |
| Estimated Fluid | Unknown | <0.1 L |
| Omit or Include | Include | Include |

Default cell feeding tasks may be returned to as needed and the expansion protocol may be continued using Feed Cells Task.

TABLE 4
Feed Cells, Modified, Example.
Table 4: Feed Cells

| Setting | Step 1 |
|---|---|
| IC Inlet | IC Media |
| IC Inlet Rate | 0.1 |
| IC Circulation Rate | −0.1 |
| EC Inlet | None |
| EC Inlet Rate | 0.00 |
| EC Circulation Rate | 100 |
| Outlet | EC Waste |
| Rocker Control | No Motion: |
| | (0°) |
| Stop Condition | Manual |
| Estimated Fluid | Unknown |
| Omit or Include | Include |

Day 4 or Later:

Resuspension of Treg Cells During Cell Culture or Prior to Harvest, Example.

A purpose of this modified Circulation Task may be to uniformly resuspend those cells that may be loosely adhered during culture or prior to initiating the Harvest Task.

In addition, this task may be used to shear Treg cell colonies every two (2) days during cell culture in order to maintain uniform cell density and nutrient diffusion beginning on or after Day 4. If the task is used to shear colonies during the culture process, the Quantum System may be returned to the modified “Feed Cells” Task.

TABLE 5
Circulation and Resuspension of Cells, Return
Cells to Bioreactor, & Feed, Example
Table 5: Custom Task 6 Settings to Resuspend Settled Cells

| Setting | Step 1 | Step 2 | Step 3 | Step 4 |
|---|---|---|---|---|
| IC Inlet | None | IC Media | IC Media | IC Media |
| IC Inlet Rate | 0 | 0.1 | 100 | 0.1 |
| IC Circulation | 300 | −0.1 | −70 | −0.1 |
| Rate | | | | |
| EC Inlet | None | None | None | None |
| EC Inlet Rate | 0 | 0 | 0 | 0 |
| EC Circulation | 100 | 100 | 100 | 100 |
| Rate | | | | |
| Outlet | EC Outlet | EC Outlet | EC Outlet | EC Outlet |
| Rocker Control | In Motion: | Stationary | In Motion: | In Motion: |
| | (−90 to | | (−90 to | (−90 to |
| | 180°) | | 180°) | 180°) |
| | Dwell Time: | | Dwell Time: | Dwell Time: |
| | 1 sec | | 1 sec | 1 sec |
| Stop Condition | Time: | Time: | IC Volume | Manual |
| | 4 min | 1 min | 150 mL | |
| Estimated Fluid | Unknown | 0.1 L | 0.2 L | Unknown |
| Omit or Include | Include | Include | Include | Include |

Harvest Quantum Harvest Task with Modification, Example.

TABLE 6
Harvest, Modification, Example
Table 6: Harvest, Modified

| Setting | Step 1 | Step 2 |
|---|---|---|
| IC Inlet | None | IC Media |
| IC Inlet Rate | 0 | 400 |
| IC Circulation Rate | 300 | −70 |
| EC Inlet | None | IC Media |
| EC Inlet Rate | 0 | 60 |
| EC Circulation Rate | 100 | 30 |
| Outlet | EC Outlet | Harvest |
| Rocker Control | In Motion: | In Motion: |
| | (−90° to 180°) | (−90° to 180°) |
| | Dwell Time: 1 sec | Dwell Time: 1 sec |
| Stop Condition | Time: | IC Volume: |
| | 4 minutes | 378 mL |
| Estimated Fluid | IC Media: <0.1 L | IC Media: 0.5 L |
| Omit or Include | Include | Include |

Harvested cells may be removed from the Quantum System by RF welding for further evaluation and analysis.

Post-Harvest Analysis

Harvested cells may be enumerated with a Vi-CELL XR 2.04 Cell Viability Analyzer (Beckman Coulter) over a range of 5-50 μm and may be quantified for membrane integrity by trypan blue dye exclusion.

Metabolism

Regulatory T cell metabolism may be monitored from the Quantum EC sample port daily by i-STAT handheld analyzer (Abbott Point of Care, Princeton, NJ) using G Cartridge for glucose and lactate concentrations using i-STAT G Cartridge (Cat. 03P83-25) and i-STAT CG4+ Cartridge (Cat. 03P85-50) respectively.

Cell Surface Biomarker Expression

Human Regulatory T cells (natural and induced) compose a small subset (2-10%) of all T cells in human cord blood and peripheral blood. Functionally, Tregs may be responsible for maintaining immunological homeostasis which may include the modulation of immune tolerance in both innate and adoptive responses. Moreover, the expression of the transcriptional regulator forkhead box P3 (FoxP3) gene product may be known to correlate with the CD4⁺CD25⁺FoxP3⁺CD127^{lo/-} Treg phenotype and the immune suppression of antigen presentation cells (APCs) and effector T cells (Teff). IL-2 binding to the CD25/IL-2 receptor (Rα) and the activation of STATS transcriptional factor may be used for Foxp3 induction. FoxP3 suppression may upregulate the activity of several genes such as CTLA-4, TNFRSF18 and IL2RA and may downregulate IL-2 via its association with histone acetylase KATS and histone deacetylase HDAC7.

Treg phenotype frequency of the harvested cell surface biomarkers may be quantified by flow cytometry. To this end, the cells may be stained with the following antibody conjugates and gated against viable, unstained cells: Fixable Viability Dye eFluor® 780 (eBioscience 65-0865), mouse anti-human CD4-PE (BD Pharmingen 561844), anti-CD4-Alexa Fluor 647 (BD Pharmingen 557707), anti-CD4-FITC (BD Pharmingen 561842), anti-CD4-FITC (BD Pharmingen 555346), anti-CD25-PE (BD Pharmingen 555432), anti-CD127-PE (BD Pharmingen 557938), anti-CD45RO-PE (BD Pharmingen 347967), and anti-FoxP3-Alexa Fluor 647 (BD Pharmingen 560045). Specimen data may be acquired on a bead-compensated BD Canto II flow cytometer equipped with FACSDiva v6.1.3 software using 1×106 cells and 20,000 total events per sample.

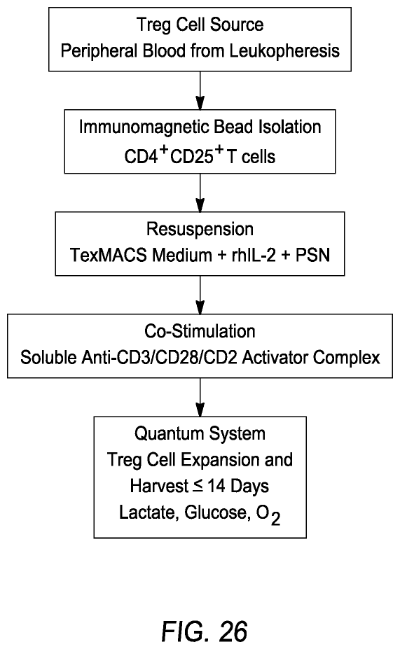

An experimental flow example is shown in FIG. 26.

Possible Results

Preliminary studies with Tregs in static culture may show that these cells tend to form micro-colonies on the order of 100 μm in diameter. Separating these cells during the medium exchange process every two days may help to limit cellular necrosis and return the cells to a high density, single cell suspension by using a 1,000 μL pipet tip that has an ID of 762 μm. Alternatively, the process of maintaining a single cell suspension may be accomplished more efficiently in an automated HFM bioreactor where the fiber lumen ID is on the order of 200 μm, such as in the Quantum System, with the aid of a preprogrammed, daily circulation task. In addition, this automated feeding task may reduce the likelihood of contamination while maintaining continuous nutrient flow to the Treg culture since it may be performed in a functionally closed system.

Possible Treg Cell Density and Viability

TABLE 7
T25 Flask Possible Harvest

| | | |
|---|---|---|
| Cell Density | 3.11 × 10⁶ | cells/mL |
| | 8.71 × 10⁵ | cells/cm2 |

| | |
|---|---|
| Viability | 81.90% |
| Stimulation Cycles | 2 |
| Doublings | 4.9 |

TABLE 8
Quantum Possible Harvest

| Cell Density | | |
|---|---|---|
| Harvest Bag | 3.61 × 10⁶ | cells/m |
| Bioreactor | 1.24 × 10⁷ | cells/mL |
| | ≥7.33 × 10⁴ | cells/cm2 |
| Viability | 84.80% | |
| Stimulation Cycles | 1 | |
| Doublings | 4.6 | |
| Doubling Time | 41.6 | hours |

Preliminary Cell Seeding Density Experiments

In preparation for the expansion of immunomagnetic selected cells from the Donor in the automated bioreactor, a series of static growth experiments may be performed to determine if stimulated Tregs may be cultured at a seeding density of less than 1.0×10⁶ cells/mL. This portion of the study may be performed by seeding 18 wells of a 24-well tissue culture plate 1.0×10⁵ cells/mL or well in TexMACS GMP medium supplemented with IL-2 (200 IU/mL) and PSN antibiotics. These cells may also be co-stimulated with the soluble anti-CD3/CD28/CD2 mAb complex on Day 0 and Day 9 at 25 μL/mL. Cells may be manually harvested and counted on Day 14 by Vi-CELL XR.

TABLE 9
Summary of possible Treg cell seeding static plate test.

| | | | | | | Treg Plate |
|---|---|---|---|---|---|---|
| | Average | Average | | | | Harvest |
| Treg | Treg | Treg | Average | | | Cell |
| Plate | Seeding | Harvest | Treg | | Treg | Viability |
| Samples | Day 0 | Day 14 | Harvest | Treg | Cell | (Trypan |
| (1 mL | (viable | (viable | Day14 | Cell | DT | Blue |
| each) | cells/mL) | cells/mL) | (cells/cm²) | DS | (hours) | Exclusion) |
| n = 18 | 1.00 × 10⁵ | 2.65 × 10⁶ | 1.33 × 10⁶ | 4.7 | 71.0 | 61.5% |
| | | SE 4.14 × 10⁴ | | | | |

Abbreviations: Doublings (DS),
Doubling Time (DT).

After harvest, the cell samples may be pooled for Treg biomarker analysis by flow cytometry. Possible results may show that the frequencies of CD4⁺C25⁺, CD4⁺CD127⁻, and CD4⁺FoxP3⁺ phenotype may be respectively 90.9%, 79.7%, and 31.6% in static culture. The CD4⁺CD25⁺ phenotype (>70%) may be generally the most reliable determinant for Treg biomarker identification since FoxP3+ detection may be highly dependent on the permeabilization method and cell viability.

The data from this static plate test may suggest that human Tregs may be expanded, with cell seeding densities on the order of 10⁵ cells/mL, when cultured in the presence of a soluble co-stimulation anti-CD3/CD28/CD2 mAb complex and serum-free medium.

Treg Metabolism

Regulatory T cells may be dependent on mitochondrial metabolism and may have the ability to oxidize multiple carbon sources, i.e., lipid or glucose. Tregs may be known to shift their metabolism from fatty acid oxidation (FAO) to glycolysis when they enter a highly proliferative state as a result of mTOR regulation of glycolysis and fatty acid metabolism. Moreover, it may have been shown that glycolysis may be necessary for the generation and suppressive functionality of human inducible Tregs by modulating the expression of FoxP3 variants through IL-2/STATS/enolase-1 promoter signaling and 2-Deoxy-D-glucose inhibition studies. Accordingly, monitoring the glucose and lactate levels may facilitate the adjustment of Quantum System media flow rates to support Treg expansion in the hollow fiber bioreactor. Initially, the Tregs may be thought to transiently reduce their metabolic rate before they enter the cell cycle and proliferate. This may be supported by the transient reduction of glycolysis and mTOR activity in freshly isolated human Tregs before TCR stimulation. Specifically, mTORC1 may be thought to increase the expression of glucose transporters such as Glut-1-mediated glucose transport as a consequence of the upregulated mTOR pathway.

The possible results of three (3) expansions from three separate Treg cell aliquots may indicate that the glucose consumption and lactate generate may appear to correlate within each Quantum System run. All three of the ex vivo expansion runs may show that glucose consumption in Tregs may increase above background levels by Day 1 and 2 out of 3 runs may show that the lactate generation levels may increase above background levels by Day 2. One run, with reduced cell viability at thaw, may generate a lagging lactate generation rate which may be reflected in the cell harvest yield. Maximum glucose consumption rates for the 2 out of 3 runs, in the most actively growing Treg cultures, may be 1.618 and 2.342 mmol/day on Days 8 and 7 respectively. The maximum lactate generation rates may be 2.406 and 3.156 mmol/day at the same time points.

Throughout the Treg expansion runs, an effort may be made to control the lactate values at ≤7 mmol/L by concurrently increasing both the IC Input (+) and IC circulation (−) pump rates from (±0.1 to ±0.4 mL/min) within the lumen of the hollow fiber membrane over Days 4-8. The lowest glucose levels during the course of the Treg cell expansions may range from 264 mg/dL on Day 7 (Q1584) to 279 mg/dL on Day 8 (Q1558). The base glucose concentration, in the cell growth formulated medium for these feasibility expansions, may be 325 to 335 mg/dL which may be found to be supportive when used in conjunction with the Quantum System flow rate adjustments.

Regulatory T Cell Biomarker Expression

The evaluation of the Treg cell harvest by flow cytometry may be centered on the CD4⁺CD25⁺FoxP3⁺ T cell subsets in this feasibility study. In T lymphocytes, the human CD4 gene, on Chromosome 12, may encode for a membrane glycoprotein which may interact with the major histocompatibility complex class II and functions to initiate the early phase of T cell activation. In regulatory T cells, the human CD25 (IL2R) gene, on Chromosome 10, encodes for the IL-2 receptor and functions by sequestering the cytokine IL-2. In regulatory T cells, the forkhead/winged-helix box P3 human gene, on the Chromosome X, may encode for the FoxP3 transcriptional factor which may be essential for Treg suppressor function. FoxP3 gene product may bind to the promoter region of CD25, CTLA-4 and IL-2, IL7R, IFN-γ genes thereby upregulating CD25 and CTLA-4 and repressing IL-2, IL7R, and IFN-γ gene transcription. The CD127 gene may encode for the IL-7 receptor and Tregs may be generally characterized by low CD127 (IL-7R) expression when compared to conventional T cells. However, certain Treg subsets may be known to express high CD127 levels during in vitro and in vivo activation which may correlate to a higher Treg survival when the cells are incubated with IL-7. The CD45RO gene product may be expressed on naive thymus derived Tregs that upon activation may lose CD45RA and express CD45RO.

TABLE 10
Possible regulatory T cell biomarker expression as a percent of parent population.

| Quantum | | | | | | |
|---|---|---|---|---|---|---|
| System | | | | | | |
| Expansion | Seeding | Harvest | CD4⁺ | CD4⁺ | CD4⁺ | CD4⁺ |
| Run | Viability | Viability | CD45RO⁺ | CD25⁺ | CD127^{low} | FoxP3⁺ |
| Q1558 | 81.9% | 84.8% | 72.4% | 86.7% | 40.1% | 58.2% |
| Q1567* | 49.6% | 69.8% | 55.3% | 79.3% | 74.2% | *5.6% |
| Q1584 | 90.7% | 94.6% | 72.8% | 90.5% | 41.0% | 64.9% |
| Average | | | | 85.5% | | *42.9% |
| (n = 3) | | | | | | |
| Average | | | | | | *61.6% |
| (n = 2)* | | | | | | |

*Low cell viability at thaw/harvest and incomplete permeabilization on Q1567 cells for FoxP3+ frequency.

The average expression of the CD4⁺CD25⁺ Treg phenotype frequency may be 85.5% in the cells harvested from the Quantum System which may compare favorably with the published CD4⁺CD25⁺ release criteria of >70%. In the Q1567 Treg expansion, the elevated frequency of the CD4⁺CD127^{low} population (74.2%) may be a reflection of the low cell viability in this particular thawed cell sample since these cells may be cultured only with IL-2 as a cytokine supplement, according to an embodiment. In cells expanded by the two Quantum System runs with seeding and harvest viability above 80%, the CD4⁺FoxP3⁺ expression frequency may be 61.6%. This finding may be consistent with the published release specification of ≥60% for FoxP3+. Furthermore, the results of the two billion cell expansions may compare favorably with the CD3⁺CD45⁺ (87.30%), CD25⁺ (47.76%), and FoxP3+(59.64%) biomarker expression in the original donor Treg cell specimen which may be received from HemaCare BioResearch Products.

Additional flow cytometry analysis may be performed on cryopreserved Treg cells from the Q1584 expansion run by a third-party laboratory, for example, using fluorescence Minus One (FMO) gating, different stains, and different instrumentation. FMO control may be a type of gating control used to interpret cell populations by taking into account the spread of all the fluorochromes in the data plots minus the one used to quantify the frequency of a particular marker. For example, the flow results from the third-party laboratory may indicate that the CD4⁺CD25⁺ Treg cell population frequency may be 95.4% from the Q1584 run which may compare favorably with the 90.5% which may be found by the Terumo BCT CES Laboratory. Incomplete staining with the alternative anti-FoxP3-PE clone stain may limit the third-party laboratory quantification of this internal biomarker, but the dot-plots may suggest that there may be a subpopulation of high expressing FoxP3⁺ Tregs in the Q1584 specimen that may not be observed in the Control Treg cell reference sample. Although interesting, additional studies may be needed to confirm these observations.

Harvest Yield

The possible average diameter of viable (trypan blue exclusion) Treg cells at Quantum System harvest may be 10.89, 11.04, and 11.06 μm respectively across the Q1558, Q1567, and Q1584 runs over a range of 5-50 μm as defined with 50 samples for each run. This may compare to an average cell diameter of 11.91, 12.40, and 7.83 μm respectively from flasks at the time of bioreactor seeding.

These possible cell diameter data may suggest that there may be more uniformity in the diameter of the cells harvested from the Quantum System than there may be in the diameter of the cells which may be expanded in the inoculum flasks.

The Treg Quantum System possible harvest data are summarized in Table 11. Moreover, the impact of the CD4⁺CD25⁺ cell viability at the point of seeding the bioreactor may be evident when comparing the results of Q1554/1584 harvests with the Q1567 harvest. There may be a 32-41% higher viability in the bioreactor inoculum for the Q1554/1584 expansion runs versus the viability for the Q1567 run. This may be due to a variation in the original cell isolation, cryopreservation technique or the length of storage since the cell aliquots that may be used in this study (HemaCare PB425C-2; Lot 14034019) may be derived from the same donor collection on Feb. 11, 2014.

TABLE 11
Possible expansion of Treg cells from inoculum flasks to Quantum System harvests.

| | | | | Treg | Treg | |
|---|---|---|---|---|---|---|
| | Treg | Treg | | DS (11 | DS (7-8 | Treg |
| | Viability | Viability | Tregs | Days) | Days) | DT (hours) |
| Quantum | Flask | Quantum | Quantum | Flask | Quantum | Quantum |
| Run | Inoculum^{A} | Harvest^{A} | Harvest^{A} | Inoculum^{B} | Harvest^{B} | Harvest^{B} |
| Q1554 | 81.9% | 84.8% | 1.82 × 10⁹ | 5.0 | 4.8 | 38.5 |
| Q1567 | 49.6% | 69.8% | 1.59 × 10⁸ | 4.4 | 1.8 | 101.3 |
| Q1584 | 90.7% | 94.6% | 1.30 × 10⁹ | 4.7 | 4.6 | 35.7 |

Abbreviations: DS-Population Doublings,
DT-Population Doubling Time in hours.
A. Harvest data may be based on Vi-Cell XR counts with Trypan Blue for membrane integrity.
B.
Note:
the Treg cell inoculum from flasks may receive two (2) rounds costimulation on Days −0 and −9; whereas the Tregs which may be harvested from the Quantum Systems may receive one (1) round of costimulation on Day −0.

The objective of this feasibility study may be to determine if the Quantum System may support the expansion of Tregs in the range of 7.0×10⁷-1.4×10⁹ cells with commercially available supplements. Two of the three bioreactor harvests from Q1554 and Q1584 may generate an average of 1.56×10⁹ Tregs, using a soluble anti-CD3/CD28/CD2 mAb co-stimulator complex, from a seeding density of <1.0×10⁶ cells/mL in less than eight (8) days. This may translate into an average harvest cell density of 8.81×10⁶ cells/mL or 7.43×10⁴ cells/cm² in the IC loop of the Quantum System bioreactor over the Q1554/1584 runs.

Possible Conclusions

The results of this feasibility study may be exploratory in nature and may not necessarily be designed to cover all technical options. For example, one may consider the reduction of inoculum Treg co-stimulation from two (2) to one (1) activation events. As such, the methods which may be used in the automated Quantum System expansion of immunomagnetic-isolated regulatory T cells may be open to modification. Our attempt here may be to define certain technical aspects of the culture process that may be conducive to further study in the upscale expansion of Tregs within the Quantum System platform. Within this context, the possible study findings may suggest that these possible conclusions or observations may be reasonable and may be helpful in the production of regulatory T cells for research, development, or production purposes.

Human Tregs, as identified as FoxP3⁺/CD25⁺, may be cultured and may be expanded with a soluble co-stimulatory anti-CD3/CD28/CD2 monoclonal antibody (mAb) T cell complex, in the absence of co-stimulatory mAb-coated beads, when supplemented with the cytokine IL-2 in the Quantum System automated hollow fiber bioreactor.

Human Tregs may be efficiently expanded in the Quantum system from cell seeding densities of less than 1×10⁶ cells/mL or less than 6.6×10⁴ cells/cm². To this end, the objective of harvesting Tregs within the range of 7.0×10⁸ to 1.4×10⁹ cells in less than 14 days may be achieved with an average (n=3) of 1.09×10⁹ total cells. An average of 85.5% of the cells may express the Treg CD4⁺CD25⁺ phenotype and an average of 42.9% may express CD4+FoxP3+ phenotype (n=3). In the two (2) billion cell Quantum System expansions, an average of 61.6% of the total cells may express the CD4⁺FoxP3⁺ phenotype. One of the three Quantum system Treg cell expansion runs may be validated for CD4⁺CD25⁺ expression by a third-party laboratory human IMSR due to the limited number of cells.

Human Tregs may be successfully cultured and may be expanded in the Quantum System by centrally seeding the cells within the lumen (IC loop) of an automated hollow fiber bioreactor.

Media IC input (+0.1 to +0.4 mL/min) and IC circulation (−0.0 to −0.4 mL/min) may be adjusted in parallel to support the Treg cell expansion process in order to maintain lactate levels≤7 mmol/L and to maintain the single cell suspension of the Treg culture by shearing cell micro-colonies at an IC circulation rate of 300 mL/min through the lumen of the Quantum System HFM bioreactor under functional closed conditions.

### Example 2

The tables below may provide example task settings (e.g., flow rates, angular rotation, outlet, etc.) for different components (e.g., pumps, rocker, valves, etc.) of a cell expansion system over several days of performing an example protocol for the expansion of T cells. The protocol may follow the following sequence:

Day 0: The Set may be loaded and primed, Media may be added, Load Cells may be added, and feeding may begin.

Day 3: A bolus of cytokines may be added to the IC loop while re-distributing the cells. Feeding may begin again.

Day 6: A bolus of cytokines may be added to the IC loop while re-distributing the cells. Feeding may begin again.

Day 9: A bolus of cytokines may be added to the IC loop while re-distributing the cells. Feeding may begin again.

Day 11-13: Cells may be Harvested; remaining cells may be reloaded. Cells may be Harvested (Day 14)

Table(s) of settings: changes made compared to example factory settings are highlighted in bold and underline

TABLE 12
IL-2 Concentration and Amount in Complete Media, Example
Complete Media

| Volume (mL) | IL-2 (IU/mL) | IL-2 (IU) |
|---|---|---|
| 2000 | 200 | 4E+05 |

TABLE 13
Volumes of Bolus Additions and IL-2 Amounts, Example
Bolus Additions

| | Volume (mL) | IL-2 (IU) |
|---|---|---|
| Day 0 (cell load) | 100 | 1E+05 |
| Day 3 | 150 | 2E+05 |
| Day 6 | 150 | 2E+05 |
| Day 9 | 150 | 4E+05 |

TABLE 14
Settings Day 0-Day 2, Example

| | | Day 0 | | | | | | | | | |
|---|---|---|---|---|---|---|---|---|---|---|---|
| | | | | | | | Load cells with Uniform | | | Cells in | |
| | | Load Cell | | | | | Suspension | | | BR | Feed cells |
| | | Expansion | | IC EC | Condition media | | (1e8 lymphocytes) | | | Custom 1 | |
| | | Set | Prime | washout | Step 1 | Step 2 | Step 1 | Step 2 | Step 3 | Step 1 | Step 2 |
| | | STEP 1 | STEP 2 | STEP 3 | STEP 4 | STEP 5 | STEP 6 | STEP 7 | STEP 8 | STEP 9 | STEP 10 |
| Task | IC inlet | Default | Default | EC media | None | None | Cell | IC | None | IC | IC |
| Settings | | settings | settings | | | | | Media | | Media | Media |
| | IC inlet | | | 100 | 0 | 0 | 25 | 25 | 0 | 100 | 0.1 |
| | rate | | | | | | | | | | |
| | IC circ rate | | | −17 | 100 | 100 | 150 | 150 | 200 | −70 | 1 |
| | EC inlet | | | EC Media | EC Media | EC Media | None | None | None | None | None |
| | EC inlet | | | 148 | 0.1 | 0.1 | 0 | 0 | 0 | 0 | 0 |
| | rate | | | | | | | | | | |
| | EC circ | | | −1.7 | 250 | 30 | 30 | 30 | 30 | 30 | 100 |
| | rate | | | | | | | | | | |
| | Outlet | | | IC and EC | EC outlet | EC outlet | EC | EC | EC | EC | EC |
| | | | | outlet | | | outlet | outlet | outlet | outlet | outlet |
| | Rocker | | | In Motion | Stationary | Stationary | In | In | In | In | Stationary |
| | | | | (−90°, | (0°) | (0°) | Motion | Motion | Motion | Motion | (0°) |
| | | | | 180°, 1 | | | (−90°, | (−90°, | (−90°, | (−90°, | |
| | | | | sec) | | | 180°, 1 | 180°, 1 | 180°, 1 | 180°, 1 | |
| | | | | | | | sec) | sec) | sec) | sec) | |
| | Stop | | | Exchange | Time (10 | Manual | Empty | IC | Time (2 | IC | Manual |
| | condition | | | (2.5 IC | min) | (20-50 | bag | volume | min) | Volume | (4320 |
| | | | | volume; | | min) | | (22 mL) | | (120 | min) |
| | | | | 2.5 EC | | | | | | mL) | |
| | | | | volume) | | | | | | | |
| Extra | Necessary | NA | 2L | 1300 mL | 6 mL/hr | | 100 mL | 22 mL | | 120 mL | 432 mL |
| Information | volume | | (PBS) | EC Media | EC Media | | | IC | | | IC |
| | | | | | | | | Media | | | Media |

| | | | | | | | |
|---|---|---|---|---|---|---|---|
| Time | 10 min | 35 min | 5 min | 30-60 min | 7 min | 2 min | 3 days |

TABLE 15
Settings Day 3-Day 5, Example

| | | Day 3 | | | | |
|---|---|---|---|---|---|---|
| | | | | Cells in BR | | Feed cells |
| | | Add Bag Contents | | Custom 2 | | |
| | | Step 1 | Step 2 | Step 1 | | Step 2 |
| | | STEP 11 | STEP 12 | STEP 13 | | STEP 14 |
| Task | IC inlet | Reagent | IC Media | IC Media | | IC Media |
| Settings | IC inlet rate | 30 | 30 | 100 | | 0.1 |
| | IC circ rate | 100 | 100 | −70 | | −0.1 |
| | EC inlet | None | None | None | | None |
| | EC inlet rate | 0 | 0 | 0 | | 0 |
| | EC circ rate | 100 | 100 | 30 | | 150 |
| | Outlet | EC outlet | EC outlet | EC outlet | | EC outlet |
| | Rocker | In Motion | In Motion | In Motion | | In Motion |
| | | (−90°, 180°, 1 | (−90°, 180°, 1 | (−90°, 180°, 1 | | (0°, 180°, 3600 |
| | | sec) | sec) | sec) | | sec) |
| | Stop | Empty Bag | IC Volume | IC Volume | | Manual |
| | condition | | (22 mL) | (120 mL) | | (4320 min) |
| Extra | Necessary | 150 mL | 22 mL IC | 120 | mL | 432 mL IC |

| | | | |
|---|---|---|---|
| Information | volume | Media | Media |

| | | | | |
|---|---|---|---|---|
| Time | 5 min | 2 | min | 3 days |

TABLE 16
Settings Day 6-Day 8, Example

| | | Day 6 | | | | | |
|---|---|---|---|---|---|---|---|
| | | | | Cells in BR | Feed cells | Feed cells | Feed cells |
| | | Add Bag Contents | | Custom 3 | | | |
| | | Step 1 | Step 2 | Step 1 | Step 2 | Step 3 | Step 4 |
| | | STEP 15 | STEP 16 | STEP 17 | STEP 18 | STEP 20 | STEP 22 |
| Task | IC inlet | Reagent | IC Media | IC Media | IC Media | IC Media | IC Media |
| Settings | IC inlet | 30 | 30 | 100 | 0.1 | 0.1 | 0.1 |
| | rate | | | | | | |
| | IC circ | 100 | 100 | −70 | −0.1 | −0.1 | −0.1 |
| | rate | | | | | | |
| | EC inlet | None | None | None | EC Media | EC Media | EC Media |
| | EC inlet | 0 | 0 | 0 | 0.1 | 0.2 | 0.3 |
| | rate | | | | | | |
| | EC circ | 100 | 100 | 30 | 200 | 200 | 250 |
| | rate | | | | | | |
| | Outlet | EC outlet | EC outlet | EC outlet | EC outlet | EC outlet | EC outlet |
| | Rocker | In Motion | In Motion | In Motion | In Motion | In Motion | In Motion |
| | | (−90° | (−90° | (−90°, 180°, | (0°, 180°, | (0°, 180°, | (0°, 180°, |
| | | 180°, 1 | 180°, 1 | 1 sec) | 3600 sec) | 3600 sec) | 3600 sec) |
| | | sec) | sec) | | | | |
| | Stop | Empty | IC Volume | IC Volume | Time | Time | Manual |
| | condition | Bag | (22 mL) | (120 mL) | (1440 min) | (1440 min) | |
| Extra | Necessary | 150 mL | 22 mL IC | 120 mL | 144 mL IC | 144 mL IC | 144 mL IC |
| Information | volume | | Media | | Media | Media | Media |
| | | | | | 144 mL EC | 288 mL EC | 436 mL EC |
| | | | | | Media | Media | Media |

| | | | |
|---|---|---|---|
| Time | 4 min | 2 min | 3 days |

TABLE 17
Settings Day 9-Day 10, Example

| | | Day 9 | | | | |
|---|---|---|---|---|---|---|
| | | | | Cells in BR | Feed cells | Feed cells |
| | | Add Bag Contents | | Custom 3 | | |
| | | Step 1 | Step 2 | Step 1 | Step 2 | Step 3 |
| | | STEP 23 | STEP 24 | STEP 17 | STEP 18 | STEP 20 |
| Task | IC inlet | Reagent | IC Media | IC Media | IC Media | IC Media |
| Settings | IC inlet rate | 30 | 30 | 100 | 0.1 | 0.1 |
| | IC circ rate | 100 | 100 | −70 | −0.1 | −0.1 |
| | EC inlet | None | None | None | EC Media | EC Media |
| | EC inlet rate | 0 | 0 | 0 | 0.1 | 0.2 |
| | EC circ rate | 100 | 100 | 30 | 200 | 200 |
| | Outlet | EC outlet | EC outlet | EC outlet | EC outlet | EC outlet |
| | Rocker | In Motion | In Motion | In Motion | In Motion | In Motion |
| | | (−90°, 180°, | (−90°, 180°, | (−90°, 180°, | (0°, 180°, | (0°, 180°, |
| | | 1 sec) | 1 sec) | 1 sec) | 3600 sec) | 3600 sec) |
| | Stop | Empty Bag | IC Volume | IC Volume | Time | Time |
| | condition | | (22 mL) | (120 mL) | (1440 min) | (1440 min) |
| Extra | Necessary | 150 mL | 22 mL IC | 120 mL | 144 mL IC | 144 mL IC |
| Information | volume | | Media | | Media | Media |
| | | | | | 144 mL EC | 288 mL EC |
| | | | | | Media | Media |

| | | | |
|---|---|---|---|
| Time | 4 min | 2 min | 2 days |

TABLE 18
Settings Day 11, Example

| | | Harvest | | | | | | |
|---|---|---|---|---|---|---|---|---|
| | | Mix | | Load cells with | | | | |
| | | Custom | | Uniform Suspension | | | Cells in BR | Feed cells |
| | | 4 | | (Harvest Product) | | | Custom 2 | |
| | | Step 1 | Harvest | Step 1 | Step 2 | Step 3 | Step 1 | Step 2 |
| | | STEP 31 | STEP 32 | STEP 33 | STEP 34 | STEP 35 | STEP 13 | STEP 14 |
| Task | IC inlet | None | EC Media | Cell | IC Media | None | IC Media | IC Media |
| Settings | IC inlet | 0 | 100 | 25 | 25 | 0 | 100 | 0.1 |
| | rate | | | | | | | |
| | IC circ | 200 | −20 | 150 | 150 | 200 | −70 | −0.1 |
| | rate | | | | | | | |
| | EC inlet | None | EC Media | None | None | None | None | None |
| | EC inlet | 0 | 100 | 0 | 0 | 0 | 0 | 0 |
| | rate | | | | | | | |
| | EC circ | 200 | 30 | 30 | 30 | 30 | 30 | 150 |
| | rate | | | | | | | |
| | Outlet | EC outlet | Harvest | EC outlet | EC outlet | EC outlet | EC outlet | EC outlet |
| | Rocker | In | In Motion | In Motion | In Motion | In Motion | In Motion | In Motion |
| | | Motion | (−90°, | (−90°, | (−90°, 180°, | (−90°, | (−90°, | (0°, 180°, |
| | | (−90°, | 180°, 1 | 180°, 1 | 1 sec) | 180°, 1 sec) | 180°, 1 sec) | 3600 sec) |
| | | 180°, 1 | sec) | sec) | | | | |
| | | sec) | | | | | | |
| | Stop | Time (3 | IC | Empty | IC volume | Time | IC Volume | Manual |
| | condition | min) | Volume | bag | (22 mL) | (2 min) | (120 mL) | (1440 min) |
| | | | (400 mL) | | | | | |
| Extra | Necessary | | 800 mL | (variable) | 22 mL | | 120 mL | 144 mL IC |
| Information | volume | | EC Media | mL | IC Media | | | Media |

| | | | | | |
|---|---|---|---|---|---|
| Time | 3 min | 4 min | 10 min | 2 min | 1 days |

TABLE 19
Settings Day 12, Example

| | | Harvest | | | | | | |
|---|---|---|---|---|---|---|---|---|
| | | Mix | | | | | | |
| | | Custom | | Load cells with Uniform Suspension | | | Cells in BR | Feed cells |
| | | 4 | | (Harvest Product) | | | Custom 2 | |
| | | Step 1 | Harvest | Step 1 | Step 2 | Step 3 | Step 1 | Step 2 |
| | | STEP 31 | STEP 32 | STEP 33 | STEP 34 | STEP 35 | STEP 13 | STEP 14 |
| Task | IC inlet | None | EC Media | Cell | IC Media | None | IC Media | IC Media |
| Settings | IC inlet | 0 | 100 | 25 | 25 | 0 | 100 | 0.1 |
| | rate | | | | | | | |
| | IC circ | 200 | −20 | 150 | 150 | 200 | −70 | −0.1 |
| | rate | | | | | | | |
| | EC inlet | None | EC Media | None | None | None | None | None |
| | EC inlet | 0 | 100 | 0 | 0 | 0 | 0 | 0 |
| | rate | | | | | | | |
| | EC circ | 200 | 30 | 30 | 30 | 30 | 30 | 150 |
| | rate | | | | | | | |
| | Outlet | EC outlet | Harvest | EC outlet | EC outlet | EC outlet | EC outlet | EC outlet |
| | Rocker | In | In Motion | In Motion | In Motion | In Motion | In Motion | In Motion |
| | | Motion | (−90°, | (−90°, | (−90°, 180°, 1 | (−90°, 180°, | (−90°, 180°, | (0°, 180°, |
| | | (−90°, | 180°, 1 | 180°, 1 | sec) | 1 sec) | 1 sec) | 3600 sec) |
| | | 180°, 1 | sec) | sec) | | | | |
| | | sec) | | | | | | |
| | Stop | Time (3 | IC | Empty bag | IC volume | Time | IC Volume | Manual |
| | condition | min) | Volume | | (22 mL) | (2 min) | (120 mL) | (1440 min) |
| | | | (400 mL) | | | | | |
| Extra | Necessary | | 800 mL | (variable) | 22 mL IC | | 120 mL | 144 mL IC |
| Information | volume | | EC Media | mL | Media | | | Media |
| | Time | 3 min | 4 min | | 10 min | | 2 min | 1 days |

TABLE 20
Settings Day 13, Example

| | | Harvest | | | | | | |
|---|---|---|---|---|---|---|---|---|
| | | Mix | | Load cells with Uniform Suspension | | | Cells in BR | Feed cells |
| | | Custom 4 | | (Harvest Product) | | | Custom 2 | |
| | | Step 1 | Harvest | Step 1 | Step 2 | Step 3 | Step 1 | Step 2 |
| | | STEP 31 | STEP 32 | STEP 33 | STEP 34 | STEP 35 | STEP 13 | STEP 14 |
| Task | IC inlet | None | EC Media | Cell | IC Media | None | IC Media | IC Media |
| Settings | IC inlet | 0 | 100 | 25 | 25 | 0 | 100 | 0.1 |
| | rate | | | | | | | |
| | IC circ | 200 | −20 | 150 | 150 | 200 | −70 | −0.1 |
| | rate | | | | | | | |
| | EC inlet | None | EC Media | None | None | None | None | None |
| | EC inlet | 0 | 100 | 0 | 0 | 0 | 0 | 0 |
| | rate | | | | | | | |
| | EC circ | 200 | 30 | 30 | 30 | 30 | 30 | 150 |
| | rate | | | | | | | |
| | Outlet | EC outlet | Harvest | EC outlet | EC outlet | EC outlet | EC outlet | EC outlet |
| | Rocker | In | In Motion | In Motion | In Motion | In Motion | In Motion | In Motion |
| | | Motion | (−90°, | (−90°, | (−90°, 180°, 1 | (−90°, 180°, | (−90°, 180°, | (0°, 180°, |
| | | (−90°, | 180°, 1 | 180°, 1 | sec) | 1 sec) | 1 sec) | 3600 sec) |
| | | 180°, 1 | sec) | sec) | | | | |
| | | sec) | | | | | | |
| | Stop | Time (3 | IC | Empty bag | IC volume | Time | IC Volume | Manual |
| | condition | min) | Volume | | (22ml) | (2 min) | (120 mL) | (1440 min) |
| | | | (400 mL) | | | | | |
| Extra | Necessary | | 800 mL | (variable) | 22 mL IC | | 120 mL | 144 mL IC |
| Information | volume | | EC Media | mL | Media | | | Media |

| | | | | | |
|---|---|---|---|---|---|
| Time | 3 min | 4 min | 10 min | 2 min | 1 days |

TABLE 21
Settings Day 14, Example

| | | Harvest | |
|---|---|---|---|
| | | Mix | |
| | | Custom 4 | |
| | | Step 1 | Harvest |
| | | STEP 31 | STEP 32 |
| Task | IC inlet | None | EC Media |
| Settings | IC inlet rate | 0 | 100 |
| | IC circ rate | 200 | −20 |
| | EC inlet | None | EC Media |
| | EC inlet rate | 0 | 100 |
| | EC circ rate | 200 | 30 |
| | Outlet | EC outlet | Harvest |
| | Rocker | In Motion | In Motion |
| | | (−90°, 180°, 1 sec) | (−90°, 180°, 1 sec) |
| | Stop condition | Time (3 min) | IC Volume |
| | | | (400 mL) |
| Extra | Necessary | | 800 mL EC Media |
| Information | volume | | |
| | Time | 3 min | 4 min |

### Example 3

Objective: The standard procedure used for coating the BioR147S bioreactor in the Quantum system may require 4-24 hours of circulation with a given adherence promoter. The 10-minute coating procedure using cryoprecipitate (CPPT) described in this document may provide an alternative for customers who desire a more expedient option for coating their bioreactors in preparation for seeding with an adherent cell type.

Preparation for the expansion of adherent cell types in the Quantum system may require the application of an adherence-promoting compound to the inner aspect of the bioreactor fibers to ensure adequate cell attachment. Two compounds may be used as coating agents for the Quantum system: fibronectin (FN) and CPPT.

CPPT is a frozen blood product prepared from fresh frozen plasma (FFP). FFP is thawed and centrifuged, and the precipitate created is collected and stored for later infusion into patients. CPPT contains fibrinogen, Von Willebrand factor, Factor VIII, and fibronectin. Although CPPT may be typically used as a therapeutic agent for patients suffering from various ailments related to blood clotting such as hemophilia or afibrinogenemia, in this case CPPT may be co-opted for use as a cell culture reagent.

During the model, CPPT solution may be introduced to the fibers of the BioR147S on the IC side. The IC waste valve may be closed, the EC waste valve may be open. IC inlet rate may be set to 50 mL/minute. In this configuration, CPPT solution may be hydrostatically deposited onto the inner wall of the bioreactor fiber for 10 minutes. This membrane ultrafiltration method may allow adherence promoting proteins to be physisorbed on the bioreactor fibers as the solution flows through the pores of the fiber from the IC to the EC side.

CPPT may be prepared in such a fashion as to create 25 mL “single donor equivalent (SDE)” aliquots:
1) Unprocessed CPPT may be obtained from a blood center;
2) CPPT may be diluted in PBS to a final volume of 100 mL for every donor represented by the product (e.g.: 5 donors for CPPT product=500 mL of total solution); and
3) This stock solution may be divided into 25 mL aliquots. Each aliquot may be sufficient to coat one Quantum bioreactor.

The 10-minute coating procedure for the Quantum System may provide a series of steps that supplant the ‘Coat Bioreactor’ portion of the standard Quantum expansion protocol (Quantum Cell Expansion System Operator's Manual for Software Version 2.0; Part No. 104101-122, pp. 10-8 to 10-10) that may be commonly used to coat the Quantum bioreactor.

This procedure may make use of the Quantum system's ability to move fluids from the IC (intracapillary) side of the bioreactor fibers to the EC (extracapillary) side via ultrafiltration through the pores of the bioreactor fibers. Rather than passively coating the bioreactor using circulating flow in the IC loop for many hours, this procedure may actively push CPPT coating solution into the IC loop and through the pores of the bioreactor, leaving a residual layer of adherence promoting proteins on the IC side of the bioreactor fibers and facilitating the attachment of adherent cells. This task may be accomplished by closing the IC waste valve and keeping the EC waste valve open allowing fluid no pathway through the bioreactor but through the pores of the fibers.

### Results:

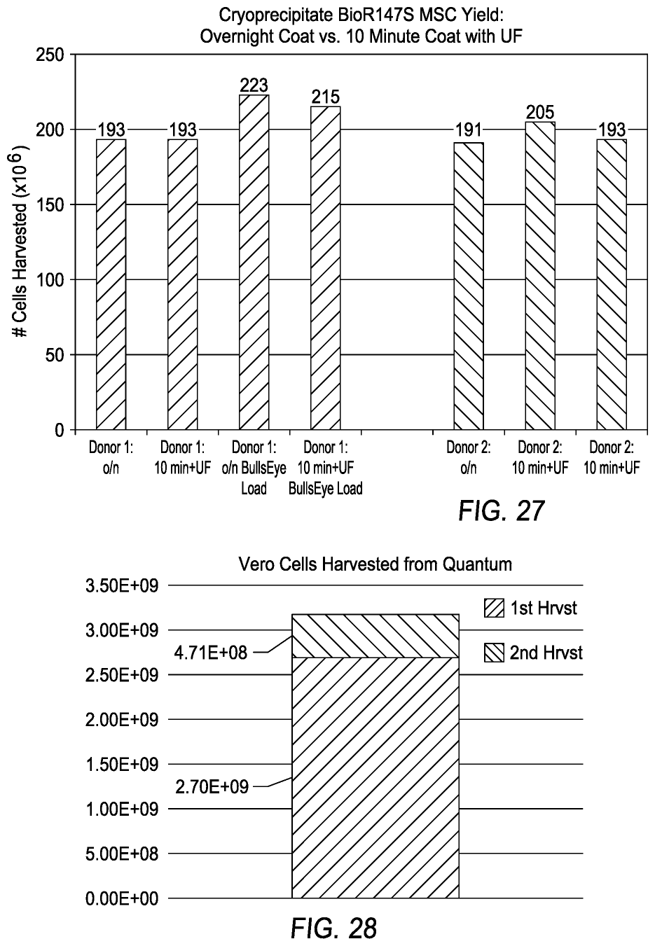

With reference to FIG. 27, at least two studies have been performed evaluating this procedure in the Quantum system for the attachment of MSC in the BioR147S. In each study, 5E+6 MSC may be loaded into a BioR147S bioreactor preconditioned with cell culture media that may be comprised of αMEM+GlutaMAX (Gibco CAT #32561102) and 10% FBS (Hyclone CAT #SH30070.03). Donor 1 MSC may be cultured for 6.8 days and donor 2 MSC may be cultured for 6.9 days.

For donor 1, n may equal 1 for both overnight-coated and 10-minute coated bioreactors. Harvest yields for donor 1 Quantum runs may be both 1.93E+8 MSC. To confirm efficacy of the 10-minute coating technique with other cell load protocols, an additional comparison may be made between Quantums loaded using the BullsEye cell load technique. MSC yield for the overnight coated/BullsEye loaded Quantum may be 2.23E+8, and MSC yield for the 10-minute coated/BullsEye loaded Quantum may be 2.15E+8.

The donor 2 MSC expansion may yield 1.91E+8 MSC from the overnight coated Quantum (n=1), and 2.05E+8 and 1.93E+8 for the 2 10-minute coated Quantums (n=2).

It should be noted that this procedure may be attempted with FN as well. Cell yields for 10-minute FN coated Quantums may be in the range of 40%-50% of overnight-coated harvests. Depending on individual needs, the 10-minute coating procedure may be a useful alternative for customers who prioritize the time-to-harvest over absolute cell yield for their particular project.

Example steps for programming the 10-minute coat with CPPT procedure into the Quantum System are shown in Table 22, below.

TABLE 22

| | Step 1 | Step 2 | Step 3 | |
|---|---|---|---|---|
| IC Inlet: (100 | Reagent | Wash | Wash | |
| mL SDE CPPT) | | | | |
| IC Inlet Rate | 10 | 10 | 60 | Proceed to |
| (mL/min) | | | | |
| IC Circ. Rate | 100 | 100 | −25 | IC/EC |
| (mL/min) | | | | |
| EC Inlet | None | None | Wash | Washout |
| EC Inlet Rate | 0 | 0 | 0.1 | Step |
| (mL/min) | | | | |
| EC Circ. Rate | 30 | 30 | 30 | |
| (mL/min) | | | | |
| Outlet | EC Outlet | EC Outlet | EC Outlet | |
| Rocker Control | Stationary | Stationary | Stationary | |
| | (0) | (0) | (0) | |
| Stop Condition | Empty Bag | IC Volume: | Time: | |
| | | 22 ml | 10.0 min | |

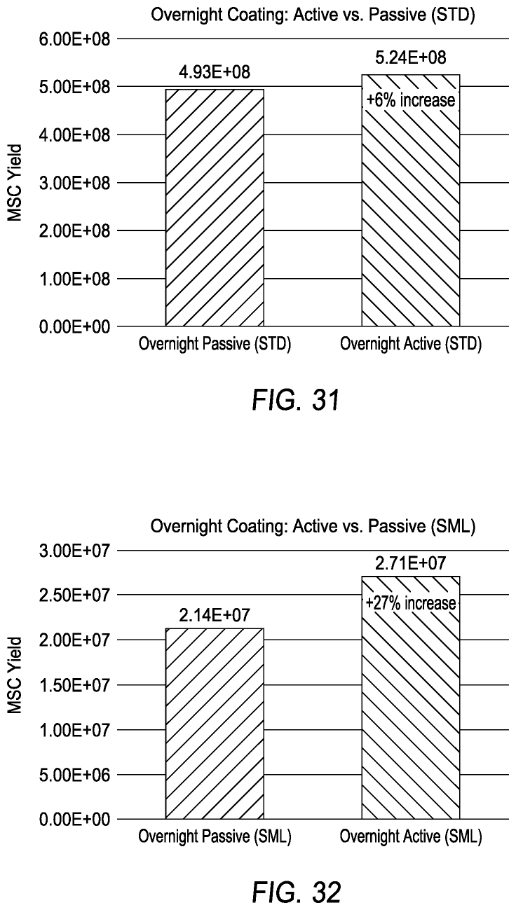

Additionally, with reference to FIGS. 31-32, the procedure for coating the bioreactor overnight with ultrafiltration, also referred to as active coating, has been evaluated. As shown in FIG. 31, overnight coating in a standard bioreactor, such as the bioreactor 100B, using active coating results in about a 6% increase in cell yield compared to passive coating. Additionally, as shown in FIG. 32, overnight coating in the small bioreactor, such as bioreactor 100A, using active coating results in about a 27% increase in cell yield compared to passive coating. The above-described results were produced with mesenchymal stem cells (MSCs).

### Example 4

Objective: The objective of this series of studies may be to determine the efficacy of the Quantum system for expansion of a cell line commonly used to produce viruses and subsequently to measure virus production from those cells in the Quantum system.

Executive Summary

Quantum may be used to culture up to 3.2 billion Vero cells at densities greater than that achieved in manual flask culture. Vero cells may be harvested and banked for future use or left attached to the Quantum hollow fiber bioreactor for viral inoculation. A maximum of 4.04E11 viral particles may be measured in the IC loop of the Quantum after 24 hours of incubation in the system. The MOI used to achieve this result may be higher than is typical, but further work may demonstrate that high viral titers such as the one demonstrated here may be reached using a more standard MOI and likely with a lower MOI. This may demonstrate that Quantum provides an environment coupled with unique fluid dynamic control that can produce virus more efficiently than current methods.

### Discussion

Vero cells may be a continuous cell line (CCL) developed by the WHO and may have been successfully used to produce viruses used in vaccine manufacture since the 1980s. Vero cells may be the most widely accepted CCL for production of both Polio and Rabies vaccines and may have been used to culture vaccines for a myriad of viruses including Dengue Fever, Chikungunya, Smallpox and West Nile Encephalitis (Barrett et al 2009). As evidenced by the ongoing outbreak of the novel Coronavirus that causes COVID-19, there may be a need for tools that could facilitate the production of multiple variations of a particular vaccine to determine the most effective variant before larger scale testing and production begins. Quantum may be seen as a potential option for use in this type of early-stage vaccine work.

In addition to being a reliable option for the culture, Quantum may have advantages over other culture systems used for viral production. The perfusion feeding mechanism and dual intracapillary (IC) and extracapillary (EC) compartments of the Quantum bioreactor may allow for a reduction of the ‘cell density effect’ seen in some viral culture systems in which viral yields per cell are significantly reduced due to the presence of high levels of toxic metabolic waste produced by infected cells as the virus propagates itself (Tapia et al, 2013). Quantum's ability to bring in fresh medium to replace spent medium as viral production proceeds may help to alleviate this problem. This process may also make use of Quantum's unique two-compartment hollow fiber environment that may use ultrafiltration across the membrane to which the host cells are bound to drive contact between suspended virus and the host cell population. This may lead to an increase in the efficiency of viral production per cell seeded due to an increased frequency of virus-host cell interaction events.

The Quantum may be seen in this context as a nimble development tool for the rapid prototyping of vaccine candidates during the development phase of vaccine production.

Results

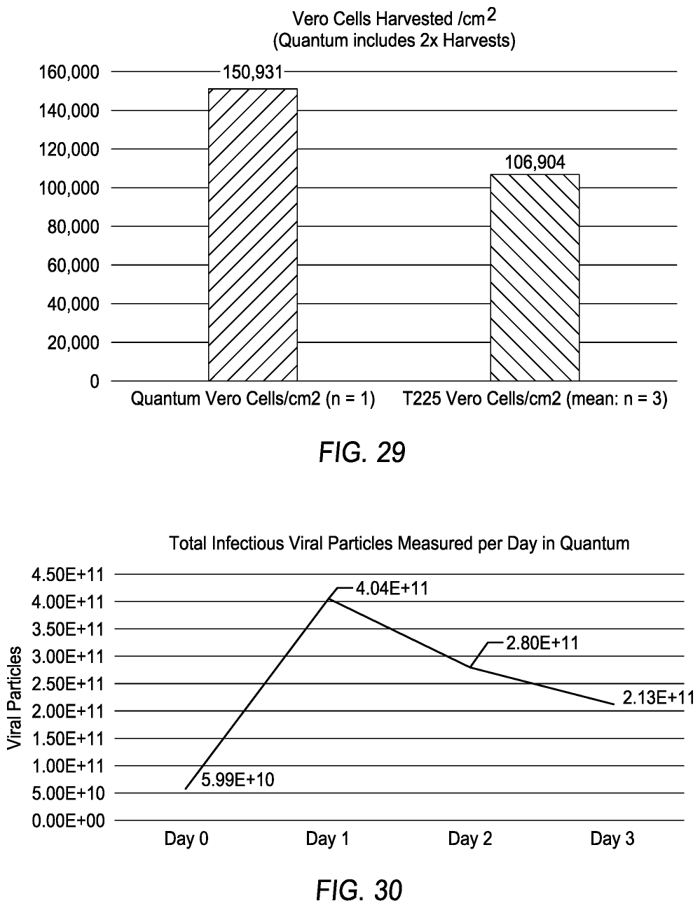

Vero Cell Culture: Initial work may focus on the expansion of viral host cells. The work may demonstrate that the TerumoBCT Quantum® system is an effective platform for the expansion of Vero cells. After coating the system overnight with 5 mg vitronectin (VN), as the harvest yield after 7 days may be approximately 3.2 billion (FIG. 28) and may be higher on a per cm2 basis than the average of 3 T225 flasks (FIG. 29). The starting number of Vero cells loaded for this run may be 25 million cells with an estimated 99% seeding efficiency as measured by post-load IC loop cell counts.

Production of Virus: The next phase of this project may involve inoculating expanded Vero cells in the Quantum with a model virus and measuring the viral yield produced from Vero cells in the Quantum system. Encephalomyocarditis (EMC) virus may be selected as the kinetics of EMC virus replication may be well defined in the protocols of TerumoBCT's pathogen reduction laboratory.

In order to maximize exposure of susceptible Vero cells to active EMC virus, a custom Quantum task may be written that may introduce the viral particles to the IC loop, may impart ultrafiltration to drive contact of virus with cells, and may allow 12 hours of time for viral incubation before the cycle of recirculate, ultrafiltrate, and incubate may be repeated every 12 hours for a total of 72 hours. A substantial overage of viral particles may be introduced to the system at a ratio of approximately 20 viral particles for each Vero cell presumed to be in the system. A final harvest of approximately 600 mL may be collected on day three after viral inoculation.

While the resulting infection curves, as determined by the TCID-50 assay, may not be seen as representative of a typical viral propagation event using a more standard multiplicity of infection (MOI) of one viral particle per 20 cells, the addition of an excess of virus may be seen as potentially defining a maximum viral production from the system operating under the assumption that it may be likely that all cells that may be infected were infected within the first 24 hours of viral propagation.

A total of 4.04E11 viral particles may be measured in the IC loop of the Quantum after a resuspension event (FIG. 30). This may be a 6.74-fold increase in viral particles relative to the number initially introduced to the system (5.99E10).

If a similar number of viral particles may be achieved using a more standard MOI of 1:20, the fold increase may be approximately 2100.

Example virus propagation steps are shown in Table 23 and Table 24, below.

TABLE 23
Day 1

| | | | UF |
|---|---|---|---|
| | Step 1 | Step 2 | Step 3 |
| IC Inlet | Cell | IC Media | IC Media |
| IC Inlet Rate | 50 | 50 | 0.2 |
| IC Circ. Rate | 0 | 0 | −0.1 |
| EC Inlet | None | None | None |
| EC Inlet Rate | 0 | 0 | 0 |
| EC Circ. Rate | 30 | 30 | 30 |
| Outlet | ECoutlet | EC outlet | EC outlet |
| Rocker Control | In Motion | In Motion | Stationary (0°) |
| | (−90°, 180°, 1 sec) | (−90°, 180°, 1 sec) | |
| Stop Condition | Empty Bag | IC volume (47 mL) | Time (30 min) |
| Media Volume (mL) | 0 | 47 | 6 |
| | Feed | Redistribute | Concentrate |
| | Step 4 | Step 5 | Step 6 |
| IC Inlet | None | None | IC Media |
| IC Inlet Rate | 0 | 0 | 10 |
| IC Circ. Rate | 0 | 200 | −5 |
| EC Inlet | EC Media | None | None |
| EC Inlet Rate | 1.3 | 0 | 0 |
| EC Circ. Rate | 1.5 | 30 | 30 |
| Outlet | EC outlet | EC outlet | EC outlet |
| Rocker Control | In Motion | (−90°, 180°, 1 sec | Stationary (0°) |
| | (−90°, 180°, 20 min) | In Motion | |
| Stop Condition | Time (720 min) | Time (2 min) | Time (12 min) |
| Media Volume (mL) | 936 | 0 | 120 |
| | Feed | Redistribute | Concentrate |
| | Step 8 | Step 9 | Step 10 |
| IC Inlet | None | None | IC Media |
| IC Inlet Rate | 0 | 0 | 10 |
| IC Circ. Rate | 0 | 200 | −5 |
| EC Inlet | EC Media | None | None |
| EC Inlet Rate | 1.3 | 0 | 0 |
| EC Circ. Rate | 30 | 30 | 30 |
| Outlet | EC outlet | EC outlet | EC outlet |
| Rocker Control | In Motion | Stationary (0°) | Stationary (0°) |
| | (−90°, 180°, 20 min) | | |
| Stop Condition | Time (720 min) | Time (5 min) | Time (12 min) |
| Media Volume (mL) | 936 | 0 | 120 |
| | Feed | Redistribute | Concentrate |
| | Step 12 | Step 13 | Step 14 |
| IC Inlet | None | None | IC Media |
| IC Inlet Rate | 0 | 0 | 10 |
| IC Circ. Rate | 0 | 200 | −5 |
| EC Inlet | EC Media | None | None |
| EC Inlet Rate | 1.3 | 0 | 0 |
| EC Circ. Rate | 30 | 30 | 30 |
| Outlet | EC outlet | EC outlet | EC outlet |
| Rocker Control | In Motion | Stationary (0°) | Stationary (0°) |
| | (−90°, 180°, 20 min) | | |
| Stop Condition | Time (720 min) | Time (5 min) | Time (12 min) |
| Media Volume (mL) | 936 | 0 | 120 |
| | Feed | Redistribute | Concentrate |
| | Step 16 | Step 17 | Step 18 |
| IC Inlet | None | None | IC Media |
| IC Inlet Rate | 0 | 0 | 10 |
| IC Circ. Rate | | 200 | −5 |
| EC Inlet | EC Media | None | None |
| EC Inlet Rate | 1.3 | 0 | 0 |
| EC Circ. Rate | 30 | 30 | 30 |
| Outlet | EC outlet | EC outlet | EC outlet |
| Rocker Control | In Motion | Stationary (0°) | Stationary (0°) |
| | (−90°, 180°, 20 min) | | |
| Stop Condition | Time (720 min) | Time (5 min) | Time (12 min) |
| Media Volume (mL) | 936 | 0 | 120 |
| | Feed | Redistribute | Concentrate |
| | Step 20 | Step 21 | Step 22 |
| IC Inlet | None | None | IC Media |
| IC Inlet Rate | 0 | 0 | 10 |
| IC Circ. Rate | 0 | 200 | −5 |
| EC Inlet | EC Media | None | None |
| EC Inlet Rate | 1.3 | 0 | 0 |
| EC Circ. Rate | 30 | 30 | 30 |
| Outlet | EC outlet | EC outlet | EC outlet |
| Rocker Control | In Motion | Stationary (0°) | Stationary (0°) |
| | (−90°, 180°, 20 min) | | |
| Stop Condition | Time (720 min) | Time (5 min) | Time (12 min) |
| Media Volume (mL) | 936 | 0 | 120 |
| | Feed | Redistribute | Concentrate |
| | Step 24 | Step 25 | Step 26 |
| IC Inlet | None | None | IC Media |
| IC Inlet Rate | 0 | 0 | 10 |
| IC Circ. Rate | 0 | 200 | |
| EC Inlet | EC Media | None | None |
| EC Inlet Rate | 1.3 | 0 | 0 |
| EC Circ. Rate | 30 | 30 | 30 |
| Outlet | EC outlet | EC outlet | EC outlet |
| Rocker Control | In Motion | Stationary (0°) | Stationary (0°) |
| | (−90°, 180°, 20 min) | | |
| Stop Condition | Time (720 min) | Time (5 min) | Time (6 min) |
| Media Volume (mL) | 936 | 0 | 120 |

Total IC Media: 726
Total EC Media: 5616

TABLE 24
Harvest
Custom Harvest

| Step 1 | Step 2 |
|---|---|
| None | EC Media |
| 0 | 100 |
| 300 | −20 |
| None | EC Media |
| 0 | 60 |
| 300 | 30 |
| EC outlet | Harvest |
| In Motion | In Motion |
| (−90°, 180°, 1 sec) | (−90°, 180°, 1 sec) |
| Time (4 min) | IC Volume (400 mL) |

### Example 5

Objective: The objective of this series of studies may be to determine the efficacy of reduced or low flow rates for both non-adherent or suspension and adherent cell types in the Quantum system. For example, flow rates between about 0.01 mL/min to about 0.1 mL/min may be used with a smaller sized cell growth chamber or bioreactor, such as the cell growth chamber 100A. In at least one example embodiment, flow rates between 0.01 mL/min to about 0.1 mL/min allow scalability between a standard bioreactor and a smaller bioreactor. For example, flow rates for a standard bioreactor, such as the bioreactor 100B, may start at about 0.1 mL/min and flow rates for the smaller bioreactor, such as the bioreactor 100A, may be reduced by one-tenth to about 0.01 mL/min.

Table 25 below may provide example task settings (e.g., flow rates, angular rotation, outlet, etc.) for different components (e.g., pumps, rocker, valves, etc.) of a small cell expansion system, such as the cell growth chamber 100A, for non-adherent or suspension cell types.

TABLE 25
Small Bioreactor

| | | | Condition Media | | Load and Position Cells | |
|---|---|---|---|---|---|---|
| | | IC/EC | | Maintain | | Chase Cells |
| | | Washout | High Flow | Condition | Load Cells | from ARC |
| Task | IC Source | IC Media | None | None | Cell | IC Media |
| Settings | IC Inlet | 100 | 0 | 0 | 5 | 5 |
| | Rate | | | | | |
| | (mL/min) | | | | | |
| | IC | −57 | 100 | 100 | 0 | 0 |
| | Circulation | | | | | |
| | Rate | | | | | |
| | (mL/min) | IC Media | EC Media | EC Media | None | None |
| | EC Source | | | | | |
| | EC Inlet | 105 | 0.1 | 0.1 | 0 | 0 |
| | Rate | | | | | |
| | (mL/min) | | | | | |
| | EC | −4.4 | 250 | 30 | 30 | 30 |
| | Circulation | | | | | |
| | Rate | | | | | |
| | (mL/min) | | | | | |
| | Outlet | IC and EC | EC Outlet | EC Outlet | EC Outlet | EC Outlet |
| | Stop | Outlet | Time (10 | Manual | Empty Bag | IC Volume |
| | Condition | Exchange | min) | | | (50 mL) |
| | | (IC Volume | | | | |
| | | 2.5 mL; EC | | | | |
| | | Volume 2.5 | | | | |
| | | mL) | | | | |
| | Rocker | In Motion | Stationary | Stationary | In Motion | In Motion |
| | | (Start −90 | (0 Deg.) | (0 Deg.) | (Start −90 | (Start −90 |
| | | Deg.; End | | | Deg.; End | Deg.; End 180 |
| | | 180 Deg.; | | | 180 Deg.; | Deg.; |
| | | Dwell Time | | | Dwell | Dwell |
| | | 1 sec.) | | | Time 1 | Time 1 sec.) |
| | | | | | sec.) | |
| | | Load and | Feed Non- | | | |
| | | Position Cells | Adherent | | | |
| | | Position | Cells | Circulate and Position Cells | | Feed Non- |
| | | Cells in | with Lowest | | Position Cells | Adherent Cells |
| | | Bioreactor | Flow Rate | Circulate Cells | in Bioreactor | with Flow Rate |
| Task | IC Source | IC Media | IC Media | None | EC Media | IC Media |
| Settings | IC Inlet | 5 | 0.02 | 0 | 5 | 0.03 |
| | Rate | | | | | |
| | (mL/min) | | | | | |
| | IC | −2.5 | −0.01 | 200 | −2.5 | −0.02 |
| | Circulation | | | | | |
| | Rate | | | | | |
| | (mL/min) | None | None | None | None | None |
| | EC Source | | | | | |
| | EC Inlet | 0 | 0 | 0 | 0 | 0 |
| | Rate | | | | | |
| | (mL/min) | | | | | |
| | EC | 30 | 30 | 100 | 30 | 100 |
| | Circulation | | | | | |
| | Rate | | | | | |
| | (mL/min) | | | | | |
| | Outlet | EC Outlet | Ec Outlet | EC Outlet | EC Outlet | EC Outlet |
| | Stop | IC Volume | Manual | Time (4 min) | IC Volume (58 | Manual |
| | Condition | (120 mL) | | | mL) | |
| | Rocker | | | | | |
| | | Deg.; End 180 | Stationary (0 | In Motion | (Start −90 Deg.; | Stationary (0 |
| | | In Motion | Deg.) | (Start −90 Deg.; | In Motion | Deg.) |
| | | (Start −90 | | End 180 Deg.; | End 180 Deg.; | |
| | | Deg.; Dwell | | Dwell Time 1 | Dwell Time 1 | |
| | | Time 1 sec.) | | sec.) | sec.) | |
| | | | | | Circulate and Position Cells | |
| | | Circulate and Position Cells | | Feed Non- | | Position |
| | | Circulate | Position Cells | Adherent Cells | Circulate | Cells in |
| | | Cells | in Bioreactor | with Flow Rate | Cells | Bioreactor |
| Task | IC Source | None | EC Media | IC Media | None | EC Media |
| Settings | IC Inlet | 0 | 5 | 0.04 | 0 | 5 |
| | Rate | | | | | |
| | (mL/min) | | | | | |
| | IC | 200 | −2.5 | −0.02 | 200 | −2.5 |
| | Circulation | | | | | |
| | Rate | | | | | |
| | (mL/min) | None | None | None | None | None |
| | EC Source | | | | | |
| | EC Inlet | 0 | 0 | 0 | 0 | 0 |
| | Rate | | | | | |
| | (mL/min) | | | | | |
| | EC | 100 | 30 | 100 | 100 | 30 |
| | Circulation | | | | | |
| | Rate | | | | | |
| | (mL/min) | | | | | |
| | Outlet | EC Outlet | EC Outlet | EC Outlet | EC Outelt | EC Outlet |
| | Stop | Time (4 min) | IC Volume (58 | Manual | Time (4 min) | IC Volume |
| | Condition | | mL) | | | (58 mL) |
| | Rocker | In Motion | In Motion | Stationary | In Motion | In Motion |
| | | (Start −90 | (Start −90 Deg.; | (0 Deg.) | (Start −90 | (Start −90 |
| | | Deg.; End | End 180 Deg.; | | Deg.; End | Deg.; End |
| | | 180 Deg.; | Dwell Time 1 | | 180 Deg.; | 180 Deg.; |
| | | Dwell Time | sec.) | | Dwell Time | Dwell Time |
| | | 1 sec.) | | | 1 sec.) | 1 sec.) |

Table 26 below, summarizes the yield and viability results of small cell expansion systems, such as the small bioreactor or cell growth chamber 100A, compared to a standard cell expansion system, such as the bioreactor 100B, for non-adherent or suspension cell types. The cells were cultured in standard (2.1 m²) bioreactors and a small (0.2 m²) bioreactors. The standard bioreactor was seeded with 30 million cells. The small bioreactors were seeded with 6 million cells. The suspension cells were re-circulated once per day. Q2320 was fed at 10% of the standard bioreactor and Q2321 was fed at 20% of the standard bioreactor. The final harvest yields and viabilities are reported in Table 26.

TABLE 26

| | Suspension | Suspension |
|---|---|---|
| Run Designation | Cell Yield | Cell Viability |
| Q2319 (Standard) | 2.71E+10 | 96% |
| Q2320 (Small - 10% of | 1.91E+09 | 90% |
| Standard) | | |
| Q2321 (Small - 20% of | 2.02E+09 | 90% |
| Standard) | | |
| Average: | 1.03E+10 | 92% |

Table 27 below, may provide example task settings (e.g., flow rates, angular rotation, outlet, etc.) for different components (e.g., pumps, rocker, valves, etc.) of a standard cell expansion system, such as the cell growth chamber 100B, for adherent cell types.

TABLE 27
Standard Clinical Bioreactor

| | | Coat Bioreactor | | | | |
|---|---|---|---|---|---|---|
| | | | Chase | | | Condition |
| | | Load | Reagent | Circulate | IC EC | Media |
| | | Reagent | from ARC | Reagent | Washout | High Flow |
| Task | IC Source | Reagent | Wash | None | IC Media | None |
| Settings | IC Inlet Rate | 10 | 10 | 0 | 100 | 0 |
| | (mL/min) | | | | | |
| | IC Circulation | 100 | 100 | 20 | 18 | 100 |
| | Rate (mL/min) | | | | | |
| | EC Source | None | None | Wash | IC Media | EC Media |
| | EC Inlet Rate | 0 | 0 | 0.1 | 148 | 0.1 |
| | (mL/min) | | | | | |
| | EC Circulation | 30 | 30 | 30 | −1.8 | 250 |
| | Rate (mL/min) | | | | | |
| | Outlet | EC Outlet | EC Outlet | EC Outlet | IC and EC | EC Outlet |
| | | | | | Outlet | |
| | Stop Condition | Empty Bag | IC Volume | Manual | Exchange (IC | Time (10 |
| | | | (22 mL) | | Volume 2.5 | min) |
| | | | | | mL; EC | |
| | | | | | Colume 2.5 mL | |
| | Rocker | Stationary | Stationary | Stationary | In Motion | Stationary |
| | | (0 Deg.) | (0 Deg.) | (0 Deg.) | (Start −90 | (0 Deg.) |
| | | | | | Deg.; End | |
| | | | | | 180 Deg.; | |
| | | | | | Dwell Time 1 | |
| | | | | | sec.) | |
| | | Condition Media | Load Cells with Uniform Suspension | | | |
| | | Maintain | | Chase Cells | Circulate | Attach |
| | | Condition | Load Cells | from ARC | Cells | Cells |
| Task | IC Source | None | Cell | IC Media | None | None |
| Settings | IC Inlet Rate | 0 | 25 | 25 | 0 | 0 |
| | (mL/min) | | | | | |
| | IC Circulation | 100 | 150 | 150 | 200 | 0 |
| | Rate (mL/min) | | | | | |
| | EC Source | EC Media | None | None | None | IC Media |
| | EC Inlet Rate | 0.1 | 0 | 0 | 0 | 0.1 |
| | (mL/min) | | | | | |
| | EC Circulation | 30 | 30 | 30 | 30 | 30 |
| | Rate (mL/min) | | | | | |
| | Outlet | EC Outlet | EC Outlet | EC Outlet | EC Outlet | EC Outlet |
| | Stop Condition | Manual | Empty Bag | IC Volume | Time (2 | Time (1440 |
| | | | | (47 mL) | min) | min) |
| | Rocker | Stationary | In Motion | In Motion | In Motion | Stationary |
| | | (0 Deg.) | (Start −90 | (Start −90 | (Start −90 | (180 Deg.) |
| | | | Deg.; End | Deg.; End | Deg.; End | |
| | | | 180 Deg.; | 180 Deg.; | 180 Deg.; | |
| | | | Dwell | Dwell Time 1 | Dwell | |
| | | | Time 1 sec.) | sec.) | Time 1 sec.) | |
| | | Feed Cells | | | | |
| | | Step 1 | Step 2 | Step 3 | Step 4 | Step 5 |
| Task | IC Source | IC Media | IC Media | IC Media | IC Media | IC Media |
| Settings | IC Inlet Rate | 0.1 | 0.2 | 0.4 | 0.8 | 1.6 |
| | (mL/min) | | | | | |
| | IC Circulation | 20 | 20 | 20 | 20 | 20 |
| | Rate (mL/min) | | | | | |
| | EC Source | None | None | None | None | None |
| | EC Inlet Rate | 0 | 0 | 0 | 0 | 0 |
| | (mL/min) | | | | | |
| | EC Circulation | 30 | 30 | 30 | 30 | 30 |
| | Rate (mL/min) | | | | | |
| | Outlet | IC Outlet | IC Outlet | IC Outlet | IC Outlet | IC Outlet |
| | Stop Condition | Time | Time | Time | Time | Manual |
| | | (2880 min) | (1440 min) | (1440 min) | (1440 min) | |
| | Rocker | Stationary | Stationary | Stationary | Stationary | Stationary |
| | | (0 Deg.) | (0 Deg.) | (0 Deg.) | (0 Deg.) | (0 Deg.) |
| | | Release Adherent Cells with Harvest | | | | |
| | | | | Chase Reagent | Circulate | Harvest |
| | | IC EC Washout | Load Reagent | from ARC | Reagent | Cells |
| Task | IC Source | Wash | Reagent | Wash | None | IC Media |
| Settings | IC Inlet Rate | 100 | 50 | 50 | 0 | 400 |
| | (mL/min) | | | | | |
| | IC Circulation | −18 | 300 | 300 | 300 | −70 |
| | Rate (mL/min) | | | | | |
| | EC Source | Wash | None | None | None | IC Media |
| | EC Inlet Rate | 148 | 0 | 0 | 0 | 60 |
| | (mL/min) | | | | | |
| | EC Circulation | −1.8 | 30 | 30 | 30 | 30 |
| | Rate (mL/min) | | | | | |
| | Outlet | IC and EC | EC Outlet | EC Outlet | EC Outlet | Harvest |
| | | Outlet | | | | Cells |
| | Stop Condition | Exchange (IC | Empty Bag | IC Volume (22 | Time (8 min.) | IC Volume |
| | | Volume 2.5 | | mL) | | (378 mL) |
| | | mL; EC | | | | |
| | | Volume 2.5 mL | | | | |
| | Rocker | In Motion | In Motion | In Motion (Start | In Motion | In Motion |
| | | (Start −90 Deg.; | (Start −90 | −90 Deg.; End | (Start −90 | (Start −90 |
| | | End 180 Deg.; | Deg.; End 180 | 180 Deg.; Dwell | Deg.; End 180 | Deg.; End |
| | | Dwell Time 1 | Deg.; Dwell | Time 1 sec.) | Deg.; Dwell | 180 Deg.; |
| | | sec.) | Time 1 sec.) | | Time 1 sec.) | Dwell |
| | | | | | | Time 1 sec.) |

Table 28 below, summarizes the yield and viability results of the standard cell expansion system, such as the cell growth chamber 100B, for adherent cell types. 30.5 million adherent cells (for Donors 1 and 2) and 31.5 million adherent cells (for Donor 3) were loaded onto each clinical bioreactor system. The final harvest yields and viabilities are reported in Table 28.

TABLE 28

| | | | MSC | MSC |
|---|---|---|---|---|
| | | Run Designation | Yield | Viability |
| Standard | Donor 1 | Q2126 | 4.96E+08 | 98% |
| Clinical | | Q2127 | 5.67E+08 | 98% |
| Bioreactor | | Donor 1 Average: | 5.31E+08 | 98% |
| | | Donor 1 Std. | | |
| | | Deviation: | 4.99E+07 | 0 |
| | Donor 2 | Q2134 | 7.22E+08 | 99% |
| | | Q2135 | 6.84E+08 | 100% |
| | | Donor 2 Average: | 7.03E+08 | 99% |
| | | Donor 2 Std. | | |
| | | Deviation: | 4.70E+07 | 0.0007 |
| | Donor 3 | Q2150 | 2.73E+08 | 98% |
| | | Q2152 | 2.83E+08 | 98% |
| | | Donor 3 Average: | 2.78E+08 | 98% |
| | | Donor 3 Std. | | |
| | | Deviation: | 7.11E+06 | 0.0014 |
| | | Total Average: | 5.04E+08 | 99% |
| | | Total Std. Deviation: | 1.93E+08 | 1% |

Table 29 below may provide example task settings (e.g., flow rates, angular rotation, outlet, etc.) for different components (e.g., pumps, rocker, valves, etc.) of a smaller-sized cell expansion system, such as the cell growth chamber 100A, for adherent cell types.

TABLE 29
Small Bioreactor

| | | | Coat Bioreactor | | | |
|---|---|---|---|---|---|---|
| | | | | Chase | | |
| | | | Load | Reagent | Circulate | IC EC |
| | | | Reagent | from ARC | Reagent | Washout |
| Task | | IC Source | Reagent | Wash | None | IC Media |
| Settings | | IC Inlet Rate | 5 | 5 | 0 | 100 |
| | | (mL/min) | | | | |
| | | IC Circulation | 50 | 50 | 6 | −57 |
| | | Rate (mL/min) | | | | |
| | | EC Source | None | None | Wash | IC Media |
| | | EC Inlet Rate | 0 | 0 | 0.1 | 105 |
| | | (mL/min) | | | | |
| | | EC Circulation | 30 | 30 | 30 | −4.4 |
| | | Rate (mL/min) | | | | |
| | | Outlet | EC Outlet | EC Outlet | EC Outlet | IC and EC |
| | | | | | | Outlet |
| | | Stop Condition | Empty Bag | IC Volume | Manual | Exchange (IC |
| | | | | (22 mL) | | Volume 2.5; |
| | | | | | | EC Volume 2.5) |
| | | Rocker | Stationary | Stationary | Stationary | In Motion |
| | | | (0 Deg.) | (0 Deg.) | (0 Deg.) | (Start −90 |
| | | | | | | Deg.; End |
| | | | | | | 180 Deg.; |
| | | | | | | Dwell Time |
| | | | | | | 1 sec.) |
| | | Condition Media | | Load Cells with Uniform Suspension | | |
| | | | Maintain | | Chase Cells | Circulate |
| | | High Flow | Condition | Load Cells | from ARC | Cells |
| Task | IC Source | None | None | Cell | IC Media | None |
| Settings | IC Inlet Rate | 0 | 0 | 5 | 5 | 0 |
| | (mL/min) | | | | | |
| | IC Circulation | 100 | 100 | 150 | 150 | 200 |
| | Rate (mL/min) | | | | | |
| | EC Source | EC Media | EC Media | None | None | None |
| | EC Inlet Rate | 0.1 | 0.1 | 0 | 0 | 0 |
| | (mL/min) | | | | | |
| | EC Circulation | 250 | 30 | 30 | 30 | 30 |
| | Rate (mL/min) | | | | | |
| | Outlet | EC Outlet | EC Outlet | EC Outlet | EC Outlet | EC Outlet |
| | Stop Condition | Time (10 | Manual | Empty Bag | IC Volume | Time (2 |
| | | min.) | | | (44 mL) | min) |
| | Rocker | Stationary | Stationary | In Motion | In Motion | In Motion |
| | | (0 Deg.) | (0 Deg.) | (Start −90 | (Start −90 | (Start −90 |
| | | | | Deg.; End | Deg.; End | Deg.; End |
| | | | | 180 Deg.; | 180 Deg.; | 180 Deg.; |
| | | | | Dwell | Dwell Time 1 | Dwell |
| | | | | Time 1 sec.) | sec.) | Time 1 sec.) |
| | | Attach | Feed Cells | | | |
| | | Cells | Step 1 | Step 2 | Step 3 | Step 4 |
| Task | IC Source | None | IC Media | IC Media | IC Media | IC Media |
| Settings | IC Inlet Rate | 0 | 0.04 | 0.08 | 0.1 | 0.2 |
| | (mL/min) | | | | | |
| | IC Circulation | 0 | 6 | 6 | 6 | 6 |
| | Rate (mL/min) | | | | | |
| | EC Source | IC Media | None | None | None | None |
| | EC Inlet Rate | 0.1 | 0 | 0 | 0 | 0 |
| | (mL/min) | | | | | |
| | EC Circulation | 30 | 30 | 30 | 30 | 30 |
| | Rate (mL/min) | | | | | |
| | Outlet | EC Outlet | IC Outlet | IC Outlet | IC Outlet | IC Outlet |
| | Stop Condition | Time (1440 | Time | Time | Time | Manual |
| | | min) | (2880 min) | (1440 min) | (1440 min) | |
| | Rocker | Stationary | Stationary | Stationary | Stationary | Stationary |
| | | (180 Deg.) | (0 Deg.) | (0 Deg.) | (0 Deg.) | (0 Deg.) |
| | | Release Adherent Cells with Harvest | | | | |
| | | | | Chase Reagent | Circulate | |
| | | IC EC Washout | Load Reagent | from ARC | Reagent | Harvest Cells |
| Task | IC Source | Wash | Reagent | Wash | None | IC Media |
| Settings | IC Inlet Rate | 100 | 5 | 5 | 0 | 400 |
| | (mL/min) | | | | | |
| | IC Circulation | −57 | 300 | 300 | 300 | −229 |
| | Rate (mL/min) | | | | | |
| | EC Source | Wash | None | None | None | IC Media |
| | EC Inlet Rate | 105 | 0 | 0 | 0 | 5 |
| | (mL/min) | | | | | |
| | EC Circulation | −4.4 | 30 | 30 | 30 | 30 |
| | Rate (mL/min) | | | | | |
| | Outlet | IC and EC | EC Outlet | EC Outlet | EC Outlet | Harvest |
| | | Outlet | | | | |
| | Stop Condition | Exchange (IC | Empty Bag | IC Volume | Time (8 min) | IC Volume |
| | | Volume 2.5; | | (22 mL) | | (150 mL) |
| | | EC Volume | | | | |
| | | 2.5) | | | | |
| | Rocker | In Motion | (Start −90 Deg.; | Deg.; End 180 | In Motion (Start | In Motion |
| | | (Start −90 Deg.; | In Motion | In Motion | −90 Deg.; End | (Start −90 |
| | | End 180 Deg.; | End 180 Deg.; | (Start −90 | 180 Deg.; Dwell | Deg.; End 180 |
| | | Dwell Time 1 | Dwell Time 1 | Deg.; Dwell | Time 1 sec.) | Deg.; Dwell |
| | | sec.) | sec.) | Time 1 sec.) | | Time 1 sec.) |

Table 30 below summarizes the yield and viability results of a small bioreactor, such as the cell growth chamber 100A, for adherent cell types. A total of 8 million adherent cells were loaded onto each small bioreactor system. The final harvest yields and viabilities are reported in Table 30.

TABLE 30

| | | | MSC | MSC |
|---|---|---|---|---|
| | | Run Designation | Yield | Viability |
| Small | Donor 1 | Q2128 | 8.77E+07 | 99% |
| Bioreactor | | Q2129 | 9.20E+07 | 97% |
| | | Q2130 | 8.72E+07 | 98% |
| | | Donor 1 Average: | 8.89E+07 | 98% |
| | | Donor 1 Std. | | |
| | | Deviation: | 2.64E+06 | 0.0068 |
| | Donor 2 | Q2131 | 1.39E+08 | 99% |
| | | Q2132 | 1.39E+08 | 99% |
| | | Q2133 | 1.26E+08 | 98% |
| | | Donor 2 Average: | 1.35E+08 | 98% |
| | | Donor 2 Std. | | |
| | | Deviation: | 7.91E+06 | 0.0052 |
| | Donor 3 | Q2151 | 5.89E+07 | 99% |
| | | Q2153 | 6.03E+07 | 99% |
| | | Q2154 | 7.03E+07 | 99% |
| | | Donor 3 Average: | 6.32E+07 | 99% |
| | | Donor 3 Std. | | |
| | | Deviation: | 6.22E+06 | 0.0026 |
| | | Total Average: | 9.56E+07 | 99% |
| | | Total Std. Deviation: | 3.18E+07 | 1% |

While example embodiments and applications of the present invention have been illustrated and described, it is to be understood that the invention is not limited to the precise configuration and resources described above. Various modifications, changes, and variations apparent to those skilled in the art may be made in the arrangement, operation, and details of the methods and systems of the present invention disclosed herein without departing from the scope of the present invention.

It will be apparent to those skilled in the art that various modifications and variations may be made to the methods and structure of the present invention without departing from its scope. Thus, it should be understood that the invention is not to be limited to the specific examples given. Rather, the invention is intended to cover modifications and variations within the scope of the following claims and their equivalents.

What is claimed is:

1. A method of expanding cells in a cell expansion system, the method comprising:
loading a first volume of fluid comprising a plurality of cells into the cell expansion system, wherein the cell expansion system comprises a cell growth chamber;
loading a second volume of fluid comprising media into a portion of a first fluid circulation path to position the first volume of fluid in a first portion of the cell growth chamber;
feeding the cells by continuously circulating media about an intracapillary circulation loop, an extracapillary circulation loop, or a combination thereof; and
expanding the cells.

2. The method of claim 1, further comprising continuously harvesting the cells.

3. The method of claim 1, wherein fluid in the first fluid circulation path flows through an intracapillary space of the cell growth chamber.

4. The method of claim 3, wherein fluid in a second fluid circulation path flows through an extracapillary space of the cell growth chamber.

5. The method of claim 1, wherein the first volume of fluid comprising the plurality of cells is loaded without activating an intracapillary circulation pump.

6. The method of claim 1, wherein the first volume of fluid is the same as the second volume of fluid.

7. The method of claim 1, further comprising:
applying a coating agent to a cell growth surface of the cell growth chamber;
wherein the coating agents includes human fibronectin, cryoprecipitate, or a combination thereof.

8. The method of claim 7, further comprising moving a fluid through a hollow fiber membrane in the cell growth chamber and into the extracapillary circulation loop using ultrafiltration.

9. The method of claim 8, wherein the fluid includes the coating agent.

10. The method of claim 1, wherein the plurality of cells include a plurality of viral cells, a plurality of Vero cells, or a combination thereof.

11. The method of claim 1, further comprising rotating the cell expansion system about a central axis using at least one rocking device.

12. A method of controlling a cell expansion system, comprising:
receiving, by a controller, data readouts from at least one sensor;
tracking and logging events, with a controller, during a cell expansion process;
recording, by the controller, data readouts from the at least one sensor; and
reporting, by the controller, events and data readouts on a display.

13. A method of controlling a fleet of cell expansion devices, comprising:
generating, by a controller, a set of instructions for the fleet of cell expansion devices, wherein the fleet of cell expansion devices includes two or more cell expansion devices;
sending, by the controller, the set of instructions for the fleet of cell expansion devices to each of the cell expansion devices in the fleet of cell expansion devices simultaneously; and
programming, by the controller, each of the cell expansion devices in the fleet of cell expansion devices with the set of instructions.

14. A method of controlling a cell expansion system, comprising:
actuating, with a controller, a first pump to flow a first fluid at a first fluid flow rate; and
actuating, with a controller, a second pump to flow a second fluid at a second fluid flow rate;
wherein the first fluid flow rate and the second fluid flow rate are opposite.

15. The method of claim 14, wherein the first fluid flow rate and the second fluid flow rate is less than 0.1 mL/min.

16. The method of claim 15, wherein the first fluid flow rate and the second fluid flow rate is 0.01 mL/min.

17. A media bag for a cell expansion system comprising:
a flexible housing defining an internal space configured to house fluid; and
a pair of ports disposed parallel within a base of the flexible housing,
wherein the flexible housing defines a hanger on a top of the internal space, opposite the pair of ports, and
the pair of ports includes an inlet port and an outlet port.

18. The media bag of claim 17, wherein the hanger includes a slot configured to receive one or more hooks for suspending the media bag.

19. The media bag of claim 17, further comprising a rod,
wherein the hanger defines a channel in the housing for receiving the rod, and
the rod and hanger are configured to fit within an external rail for suspending the media bag.

20. A cell expansion system comprising:
a cell expansion device including a housing and a holder, the housing defining a receiver on an operational face thereof, and the holder projecting from the housing;
a disposable set configured to be engaged with the receiver on the cell expansion device; and
a media bag configured to be suspended on the holder of the cell expansion device, wherein
the media bag has a flexible housing defining an internal space configured to house fluid, the flexible housing defining a hanger on a top of the internal space, the hanger including a rod extending longitudinally along the top of the media bag, and
the rod is configured to be received within a channel on the holder to secure the media bag to the holder on the cell expansion device.

21. The cell expansion device of claim 20, wherein the disposable set includes a cell growth chamber and tubing, the tubing being configured to connect the cell growth chamber to the media bag.

22. The cell expansion device of claim 20, wherein
the holder includes at least one projection, and
the hanger defines slots configured to receive the projection and suspend the media bag.

23. A method of controlling a cell expansion system, comprising:
actuating, with a controller, a first pump to flow a first fluid at a first fluid flow rate;
actuating, with a controller, a second pump to flow a second fluid at a second fluid flow rate;
detecting, with a sensor, a parameter related to the first fluid or the second fluid;
receiving, by a controller, the parameter from the sensor and determining whether the parameter is outside of a predetermined range; and
sending, by a controller, a remote alarm to an external device separate from the cell expansion system.

24. The method of claim 23, wherein the remote alarm comprises an email, a text message, a digital alert, or a combination thereof.

25. The method of claim 23, further comprising activating an audio alarm, a visual alarm, or a combination thereof.

26. The method of claim 23, wherein the sensor comprises a thermistor, pressure sensor, gas sensor, air detector, metabolic sensor or combination thereof.

27. A method of controlling a cell expansion system, comprising:
detecting a parameter related to a cell expansion device;
receiving, by a controller, the parameter and determining whether the parameter is outside of a predetermined range; and
sending, by a controller, a remote alarm to an external device separate from the cell expansion system.

28. The method of claim 27, wherein the remote alarm comprises an email, a text message, a digital alert, or a combination thereof.

29. The method of claim 27, further comprising activating an audio alarm, a visual alarm, or a combination thereof.

30. The method of claim 27, wherein the parameter is one of a temperature, a door position, a pressure, a flow rate, and a concentration.
